SEQUENCE LISTING (58 sequences)

SEQ ID NO: 1 - DNA, 13 bp, ARTIFICIAL
  OLIGONUCLEOTIDE
ccgaccagccttg

SEQ ID NO: 2 - DNA, 19 bp, ARTIFICIAL
  OLIGONUCLEOTIDE
aaagctcaaggctggtcgg

SEQ ID NO: 3 - RNA, 12 nt, ARTIFICIAL
  OLIGONUCLEOTIDE
uuuugccgcgcc

SEQ ID NO: 4 - DNA, 16 bp, ARTIFICIAL
  OLIGONUCLEOTIDE
ggaatgctggcgcggc

SEQ ID NO: 5 - DNA, 23 bp, ARTIFICIAL
  OLIGONUCLEOTIDE
ttaaaagaaaaggggggactgga

SEQ ID NO: 6 - DNA, 57 bp, ARTIFICIAL
  OLIGONUCLEOTIDE
cgttgggagtgaattagcccttccagtccccccttttcttttaaaaagtggctaaga

SEQ ID NO: 7 - DNA, 34 bp, ARTIFICIAL
  OLIGONUCLEOTIDE
gcacaaattaatcgtctgttgcttatcaactcac

SEQ ID NO: 8 - DNA, 34 bp, ARTIFICIAL
  OLIGONUCLEOTIDE
gtgagttgataagcaacagacgattaatttgtgc

SEQ ID NO: 9 - DNA, 7433 bp, Human Poliovirus
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttggttagcactcaaccccagagtgtagcttaggctgatgagtctggacatccctcaccggtgacggtgttccaggctgcgttggcggcctacctatggctaacgcatgggacgctagttgtgaacaaggtgtgaagagcctattgagctacataagaatcctccggcccctgaatgcggctaatcccaacctcggagcaggtggttcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatgggtgctcaggtttcatcacagaaagtgggcgcacatgaaaactcaaatagagcgtatggtggttctaccattaattacaccaccattaattattatagagattcagctagtaacgcggcttcgaaacaggacttctctcaagacccttccaagttcaccgagcccatcaaggatgtcctgataaaaacagccccaatgctaaactcgccaaacatagaggcttgcgggtatagcgatagagtactgcaattaacactgggaaactccactataaccacacaggaggcggctaattcagtagtcgcttatgggcgttggcctgaatatctgagggacagcgaagccaatccagtggaccagccgacagaaccagacgtcgctgcatgcaggttttatacgctagacaccgtgtcttggacgaaagagtcgcgagggtggtggtggaagttgcctgatgcactgagggacatgggactctttgggcaaaatatgtactaccactacctaggtaggtccgggtacaccgtgcatgtacagtgtaacgcctccaaattccaccagggggcactaggggtattcgccgtaccagagatgtgtctggccggggatagcaacaccactaccatgcacaccagctatcaaaatgccaatcctggcgagaaaggaggcactttcacgggtacgttcactcctgacaacaaccagacatcacctgcccgcagttctgcccggtggattacctccttggaaatggcacgttgttggggaatgcctttgtgttccgcacagataataaacttacggaccaacaactgtgctacactggtactaccttacgtgaactccctctcgctagatagtatggtaaagcacaataattggggaattgcaatattaccattggccccattaaattttgttagtgagtcctccccagagattccaatcaccttgaccatagcccctatgtgctgtgagttcaatggattaagaaacatcaccctgccacgcttacagggcctgccggtcatgaacacccctggtagcaatcaatatcttactgcagacaacttccagtcaccgtgtgcgctgcctgaatttgatgtgaccccacctattgacatacccggtgaagtaaagaacatgatggaattggcagaaatcgacaccatgattccctttgacttaagtgccacaaaaaagaacaccatggaaatgtatagggttcggttaagtgacaaaccacatacagccgcctccatactctgcctgtcactctctccagcctcagatcctaggttgtcacatactatgcttggagaaatcctaaattactacacacactgggcaggatccctgaagttcacgtttctgttctgtggatccatgatggcaactggcaaactgttggtgtcatacgcgcctcctggagccgacccaccaaagaagcgtaaggaggcgatgttgggaacacatgtgatctgggacataggactgcagtcctcatgtactatggtagtgccatggattagcaacagcacgtatcggcaaaccatagatgatagtttcaccgaaggcggatacatcagcgtcttctaccaaactagaatagtcgtccctctttcgacacccagagagatggacatccttggttttgtgtcagcgtgtaatgacttcagcgtgcgcttgttgcgagataccacacatatagagcaaaaagcgctagcacaggggttaggtcagatgcttgaaagcatgattgacaacacagtccgtgaaacggtgggggcggcaacatctagagacgctctcccaaacactgaagccagtggaccaacacactccaaggaaattccggcactcaccgcagtggaaactggggccacaaatccactagtcccttctgatacagtgcaaaccagacatgttgtacaacataggtcaaggtcagagtctagcatagagtctttcttcgcgcggggtgcatgcgtgaccattatgaccgtggataacccagcttccaccacgaataaggataagctatttgcagtgtggaagatcacttataaagatactgtccagttacggaggaaattggagttcttcacctattctagatttgatatggaacttacctttgtggttactgcaaatttcactgagactaacaatgggcatgccttaaatcaagtgtaccaaattatgtacgtaccaccaggcgctccagtgcccgaaaaatgggacgactacacatggcaaacctcatcaaatccatcaatcttttacacctacgggacagctccagcccggatctcggtaccgtatgttggtatttcgaacgcctattcacacttttacgacggtttttccaaagtaccactgaaggaccagtcggcagcactaggtgactccctttatggtgcagcatctctaaatgacttcggtattttggctgttagagtagtcaatgatcacaacccgaccaaggtcacctccaaaatcagagtgtatctaaaacccaaacacatcagagtctggtgcccgcgtccaccgaggcagctggcgtactacggccctggagtggattacaaggatggtacgcttacacccctctccaccaaggatctgaccacatatggattcggacaccaaaacaaagcggtgtacactgcaggttacaaaatttgcaactaccacttggccactcaggatgatttgcaaaacgcagtgaacgtcatgtggagtagagacctcttagtcacagaatcaagagcccagggcaccgattcaatcgcaaggtgcaattgcaacgcaggggtgtactactgcgagtctagaaggaaatactacccagtatccttcgttggcccaacgttccagtacatggaggctaataactattacccagttaggtaccagtcccatatgctcattggccatggattcgaatctccaggggattgtggtggcatactcagatgtcaccacggggtgatagggatcattactgctggtggagaagggttggttgcattttcagacattagagacttgtatgcctacgaagaagaagccatggaacaaggcatcaccaattacatagagtcacttggggccgcatttggaagtggatttactcagcagattagcgacaaaataacagagttgaccaatatggtgaccagtaccatcactgaaaagctacttaagaacttgatcaagatcatatcctcactagttattataactaggaactatgaagacaccacaacagtgctcgctaccctggcccttcttgggtgtgatgcttcaccatggcagtggcttagaaagaaagcatgcgatgttctggagataccttatgtcatcaagcaaggtgacagttggttgaagaagtttactgaagcatgccaacgagctaagggcctggagtgggtgtcaaacaaaatctcaaaattcattgattggctcaaggagaaaattatcccacaagctagagataagttggaatttgtaacaaaacttagacaactagaaatgctggaaaaccaaatctcaactatacaccaatcatgccctagtcaggaacaccaggaaattctattcaataatgtcagatggttatccatccagtctaagaggtttgcccctctttacgcagtggaagccaaaagaatacagaaactagagcatactattaacaactacatacagttcaagagcaaacaccgtattgaaccagtatgtttgctagtacatggcagccccggaacaggtaaatctgtagcaaccaacctgattgctagagccatagctgaaagagaaaacacgtccacgtactcgctacccccggatccatcacacttcgacggatacaaacaacagggagtggtgattatggacgacctgaatcaaaacccagatggtgcggacatgaagctgttctgtcagatggtatcaacagtggagtttataccacccatggcatccctggaggagaaaggaatcctgtttacttcaaattacgttctagcatccacaaactcaagcagaatttccccccccactgtggcacacagtgatgcattagccaggcgctttgcgttcgacatggacattcaggtcatgaatgagtattctagagatgggaaattgaacatggccatggctactgaaatgtgtaagaactgtcaccaaccagcaaactttaagagatgctgtcctttagtgtgtggtaaggcaattcaattaatggacaaatcttccagagttagatacagtattgaccagatcactacaatgattatcaatgagagaaacagaagatccaacattggcaattgtatggaggctttgtttcaaggcccactccagtataaagacttgaaaattgacatcaagacgagtccccctcctgaatgtatcaatgacttgctccaagcagttgactcccaggaggtgagagattactgtgagaagaagggttggatagtcaacatcaccagccaggttcaaacagaaaggaacatcaacagggcaatgacaattctacaagcggtgacaaccttcgccgcagtggctggagttgtctatgtcatgtataaactgtttgctggacaccagggagcatacactggtttaccaaacaaaaaacccaacgtgcccaccattcggacagcaaaggtacaaggaccagggttcgattacgcagtggctatggctaaaagaaacattgttacagcaactactagcaagggagagttcactatgttaggagtccacgacaacgtggctattttaccaacccacgcttcacctggtgaaagcattgtgatcgatggcaaagaagcggagatcttggatgccaaagtgtttgaagatcaagcaggaaccaattttgaaatcactataatcactctaaagagaaatgaaaagttcagagacattagaccacatatacctactcaaatcactgagacaaatgatggagtcttgatcgtgaacactagcaagtaccccaatatgtatgttcctgtccgtgctgtgactgaacagggatatctaaatctcggtgggcgccaaactgctcgtactctaatgtacaactttccaaccagagcaggacagtgtggtggagtcatcacatgtactgggaagtcatcgggatgcatgttggtggacggttcacacgggtttgcagcggccctgaagcgatcattattcactcagagtcaaggtgaaatcccgtggatgagaccttcgaaggacgcgggatatccaatcataaatgccccgtccaaaaccaagcttgaacccagtgctttccactatgtgtttgaaggggtgaaggaaccagcagtcctcactaaaaacgatcccaggcttaagacagactttgaggaggcaattttctccaagtacgtgggtaacaaaattactgaagtggatgagtacatgaaagaggcagtagaccactatgctggccagctcatgtcactagacatcaacatagaacaaatgtgcttggaggatgccatgtatggcactgatggtctagaagcacttgatttgtccaccagtgctggctacccttatgtagcaatgggaaagaagaagagagacatcttgaacaaacaaaccagagacactaaggaaatgcaaaaactgctcgacacatatggaatcaacctcccactggtgacttatgtaaaggatgaacttagatccaaaacaaaggttgagcaggggaaatccagattaattgaagcttctagtttgaatgactcagtggcaatgagaatggcttttgggaacctatatgctgcttttcacaaaaacccaggagtgataacaggttcagcagtggggtgcgatccagatttgttttggagcaaaattccggtattgatggaagagaagctgtttgcttttgactacacagggtatgatgcatctctcagccctgcttggttcgaggcactaaagatggtgcttgagaaaatcggattcggagacagagttgactacatcgactacctaaaccactcacaccacctgtacaagaataaaacatactgtgtcaagggcggtatgccatctggctgctcaggcacttcaatttttaactcaatgattaacaacttgattatcaggacactcttactgaaaacctacaagggcatagatttagaccacctaaaaatgattgcctatggtgatgatgtaattgcttcctacccccatgaagttgacgctagtctcctagcccaatcaggaaaagactatggactaactatgactccagctgacaaatcagctacatttgaaacagtcacatgggagaatgtaacattcttgaagagattcttcagggcagacgagaaatacccatttcttattcatccagtaatgccaatgaaggaaattcatgaatcaattagatggactaaagatcctaggaacactcaggatcacgttcgctctctgtgccttttagcttggcacaatggcgaagaagaatataacaaattcctagctaaaatcaggagtgtgccaattggaagagctttattgctcccagagtactcaacattgtaccgccgttggcttgactcattttagtaaccctacctcagtcgaattggattgggtcatactgctgtaggggtaaatttttctttaattcggagg

SEQ ID NO: 10 - protein, 757 aa, Influenza A virus
MDVNPTLLFLKVPAQNAISTTFPYTGDPPYSHGTGTGYTMDTVNRTHQYSEKGKWTTNTETGAPQLNPIDGPLPEDNEPSGYAQTDCVLEAMAFLEESHPGIFENSCLETMEVVQQTRVDKLTQGRQTYDWTLNRNQPAATALANTIEVFRSNGLTANESGRLIDFLKDVMESMDKEEMEITTHFQRKRRVRDNMTKKMVTQRTIGKKKQRVNKRGYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYITKNQPEWFRNILSIAPIMFSNKMARLGKGYMFESKRMKLRTQIPAEMLANIDLKYFNESTRKKIEKIRPLLIDGTASLSPGMMMGMFNMLSTVLGVSILNLGQKKYTKTTYWWDGLQSSDDFALIVNAPNHEGIQAGVDRFYRTCKLVGINMSKKKSYINKTGTFEFTSFFYRYGFVANFSMELPSFGVSGINESADMSIGVTVIKNNMINNDLGPATAQMALQLFIKDYRYTYRCHRGDTQIQTRRSFELKKLWDQTQSRAGLLVSDGGPNLYNIRNLHIPEVCLKWELMDENYRGRLCNPLNPFVSHKEIESVNNAVVMPAHGPAKSMEYDAVATTHSWIPKRNRSILNTSQRGILEDEQMYQKCCNLFEKFFPSSSYRRPIGISSMVEAMVSRARIDARIDFESGRIKKEEFSEIMKICSTIEELRRQK

SEQ ID NO: 11 - protein, 554 aa, Human immunodeficiency virus type 1
KISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDICKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNKGRQKVVPLTDTTNQKTQLQAIYLALQDSGLEVNIVTDSQYALGIIQAQPDESESELVNQIIEQLIKKEKVYLAWVPAHKGIGGNEQVDKLVSA

SEQ ID NO: 12 - RNA, 10 nt, ARTIFICIAL SEQUENCE
  synthetic construct
gcuagggccc

SEQ ID NO: 13 - RNA, 10 nt, ARTIFICIAL SEQUENCE
  synthetic construct
gcungggccc

SEQ ID NO: 14 - RNA, 10 nt, ARTIFICIAL SEQUENCE
  synthetic construct
gcnugggccc

SEQ ID NO: 15 - RNA, 10 nt, ARTIFICIAL SEQUENCE
  synthetic construct
cngugggccc

SEQ ID NO: 16 - protein, 28 aa, Influenza A virus
NRFYRTCKLLGINMSKKKSYINRTGTFE

SEQ ID NO: 17 - protein, 28 aa, Influenza B virus
NDFYRTCKLLGINMSKKKSYCNETGMFE

SEQ ID NO: 18 - protein, 28 aa, Influenza C virus
RRFNAVCKLIGINMSLEKSYGSLPELFE

SEQ ID NO: 19 - protein, 29 aa, Poliovirus
SLLAQSGKDYGLTMTPADKSATFETVTWE

SEQ ID NO: 20 - protein, 27 aa, Poliovirus
VDASLLAQSGKDYGLTMTPADKSATFE

SEQ ID NO: 21 - protein, 27 aa, coxsackievirus
VDASLLAQSGKDYGLTMTPADKSATFE

SEQ ID NO: 22 - protein, 27 aa, human rhinovirus 14
LDPQVLATLGKNYGLTITPPDKSETFT

SEQ ID NO: 23 - protein, 29 aa, rabbit haemorrhagic disease virus
MVSLLPAIIENLRDYGLSPTAADKTEFID

SEQ ID NO: 24 - protein, 30 aa, feline calicivirus
FASVSDQIFANLSAYGLKPTRVDKSVGSIE

SEQ ID NO: 25 - protein, 35 aa, severe acute respiratory syndrome coronavirus
YAAQGLVASIKNFKAVLYYQNNVFMSEAKCWIETD

SEQ ID NO: 26 - protein, 35 aa, mouse hepatitis virus
FASKGYIANISAFQQVLYYQNNVFMSEAKCWVETD

SEQ ID NO: 27 - protein, 34 aa, hepatitis C virus
GTQEDEASLRAFTEAMTRYSAPPGDPPKPEYDLE

SEQ ID NO: 28 - protein, 29 aa, human immunodeficiency virus type-1
IGQHRTKIEELRQHLLRWGLTTPDKKHQK

SEQ ID NO: 29 - protein, 22 aa, bacteriophage Q-beta
PALREVFKYVGFTTNTKKTFSE

SEQ ID NO: 30 - protein, 12 aa, Moloney murine leukemia virus
GYRASAKKAQIC

SEQ ID NO: 31 - protein, 11 aa, Human immunodeficiency virus type 1
GLTTPDKKHQK

SEQ ID NO: 32 - protein, 11 aa, Human immunodeficiency virus type 2
GFSTPDEKFQK

SEQ ID NO: 33 - protein, 11 aa, Tribolium castaneum
GVYEVPTKTRT

SEQ ID NO: 34 - protein, 14 aa, poliovirus
GLTMTPADKSATFE

SEQ ID NO: 35 - protein, 15 aa, Foot-and-mouth disease virus
GQTITPADKSDKGFV

SEQ ID NO: 36 - protein, 14 aa, human rhinovirus type 14
GLTITPPDKSEGFT

SEQ ID NO: 37 - protein, 14 aa, human rhinovirus type 16
GLTITPADKSSEFK

SEQ ID NO: 38 - protein, 14 aa, human rhinovirus type 1B
GLTITPADKSTEFK

SEQ ID NO: 39 - protein, 14 aa, rabbit hemorrhagic disease virus
GLSPTAADKTEFID

SEQ ID NO: 40 - protein, 15 aa, Norwalk virus
GLKPTRPDKTEGPLV

SEQ ID NO: 41 - protein, 15 aa, hepatitis C virus
SAPPGDPPKPEYDLE

SEQ ID NO: 42 - protein, 11 aa, bovine viral diarrhea virus
GKPQKIDTTGE

SEQ ID NO: 43 - protein, 15 aa, Dengue virus
GKVRKDIPQWQPSKW

SEQ ID NO: 44 - protein, 17 aa, West Nile Virus
SKVRKDIQEWKPSTGWY

SEQ ID NO: 45 - DNA, 35 bp, ARTIFICIAL
  OLIGONUCLEOTIDE
atctggctgctcaggtacctcaatttttaactcaa

SEQ ID NO: 46 - DNA, 37 bp, ARTIFICIAL
  OLIGONUCLEOTIDE
gcggaattcggatccttactaaaatgagtcaagccaa

SEQ ID NO: 47 - DNA, 36 bp, ARTIFICIAL
  OLIGONUCLEOTIDE
atgactccagctgacctgtcagctacatttgaaaca

SEQ ID NO: 48 - DNA, 36 bp, ARTIFICIAL
  OLIGONUCLEOTIDE
tgtttcaaatgtagctgacaggtcagctggagtcat

SEQ ID NO: 49 - DNA, 36 bp, ARTIFICIAL
  OLIGONUCLEOTIDE
atgactccagctgaccgttcagctacatttgaaaca

SEQ ID NO: 50 - DNA, 36 bp, ARTIFICIAL
  OLIGONUCLEOTIDE
tgtttcaaatgtagctgaacggtcagctggagtcat

SEQ ID NO: 51 - DNA, 36 bp, ARTIFICIAL
  OLIGONUCLEOTIDE
atgactccagctgaccactcagctacatttgaaaca

SEQ ID NO: 52 - DNA, 36 bp, ARTIFICIAL
  OLIGONUCLEOTIDE
tgtttcaaatgtagctgagtggtcagctggagtcat

SEQ ID NO: 53 - DNA, 32 bp, ARTIFICIAL
  OLIGONUCLEOTIDE
ccacaccagacaaactacatcagaaagaacct

SEQ ID NO: 54 - DNA, 32 bp, ARTIFICIAL
  OLIGONUCLEOTIDE
aggttctttctgatgtagtttgtctggtgtgg

SEQ ID NO: 55 - DNA, 34 bp, ARTIFICIAL; identical to SEQ ID NO: 7
  OLIGONUCLEOTIDE

SEQ ID NO: 56 - DNA, 34 bp, ARTIFICIAL; identical to SEQ ID NO: 8
  OLIGONUCLEOTIDE

SEQ ID NO: 57 - DNA, 36 bp, ARTIFICIAL
  OLIGONUCLEOTIDE
actcgcagtgtgactctgcgttcagtcatgacgctg

SEQ ID NO: 58 - DNA, 39 bp, ARTIFICIAL
  OLIGONUCLEOTIDE
cagcgtcatgactgaacgcagagtcacactgcgagtaac

(19) United States
(12) Patent Application Publication
(10) Pub. No.: US 2010/0129400 A1
(43) Pub. Date: May 27, 2010

(54) ATTENUATED VIRUSES, VACCINES AND METHODS OF USE THEREOF

(75) Inventors: Craig Cameron, State College, PA (US); Jamie Arnold, State College, PA (US); Avery August, State College, PA (US)

Correspondence Address: J. Rodman Steele;Novak Druce & Quigg LLP, 525 Okeechobee Blvd, Suite 1500, West Palm Beach, FL, 33401, US

(73) Assignee: THE PENN STATE RESEARCH FOUNDATION, University Park, PA (US)

(21) Appl. No.: 12/686,200

(22) Filed: Jan. 12, 2010

Related U.S. Application Data
(63) Continuation-in-part of application No. 11/963,930, filed on Dec. 24, 2007.
(60) Provisional application No. 60/871,570, filed on Dec. 22, 2006.
(60) Provisional application No. 60/892,086, filed on Feb. 28, 2007.
(60) Provisional application No. 61/144,334, filed on Jan. 13, 2009.

(51) Int. Cl. A61K 39/21; A61K 39/12; A61K 39/145; A61K 39/13; A61P 37/00; A61P 31/12; A61P 31/18; A61P 31/16

(52) U.S. Cl. 424/208.1; 424/204.1; 424/209.1; 424/217.1

(57) ABSTRACT

The invention encompasses compositions and methods relating to viral polymerases having one or more substitutions of different amino acids at conserved regions of the polymerase yields enzymes with varying rates and fidelity of replication. A universally applicable, polymerase-mechanism-based strategy for production of attenuated viruses and anti-viral vaccines is disclosed. Attenuated viruses serve as vaccines and were shown to provide protection against poliovirus challenge in vivo.

### CROSS REFERENCE TO RELATED APPLICATION

The present application is a continuation-in-part application of U.S. patent application Ser. No. 11/963,930 filed Dec. 24, 2007 that claims the priority of U.S. provisional patent application No. 60/871,570 filed Dec. 22, 2006, and U.S. provisional patent application No. 60/892,086 filed Feb. 28, 2007. The present application also claims priority from U.S. provisional patent application No. 61/144,334 filed Jan. 13, 2009. Applicants herein claim the benefit of all these prior patent applications, the entire contents of which are incorporated herein by reference.

### STATEMENT AS TO FEDERALLY SPONSORED RESEARCH

This invention was made with government support under grant number AI45818, awarded by the National Institutes of Health. The government has certain rights in the invention.

### FIELD OF THE INVENTION

The invention relates generally to the fields of molecular biology, virology and immunology. More particularly, the invention relates to viral attenuation and anti-viral vaccines.

### BACKGROUND

Many successful viral vaccines use live attenuated virus strains. The rate-limiting step for live-virus vaccine development is the identification of a suitable attenuated virus. Conventional methods of developing an attenuated virus involve propagation of the virus under novel conditions, such as passage of the virus in “foreign” or non-permissive cell lines, so that it becomes less pathogenic to its original host as it evolves under the new conditions. Although this methodology has shown remarkable success, little is known about the process by which the attenuating mutations arise and evolve, and isolating an attenuated virus is a random, slow process. In addition, the outcome of an attempted attenuation is largely unpredictable, and depending on the nature of the attenuation, an attenuated virus may revert to virulence.

Some currently used attenuation methods result in virus vaccine strains that are too virulent to produce. Highly virulent strains are deleterious to the host and developing vaccines requires inactivation of the virus. This has certain drawbacks. For example, inactivated viruses as orally or nasally applied vaccines must be given in high concentrations in order to bring about a significant increase of antibodies. As another example, the administration of inactivated influenza virus or antigen in convenient commercial doses, free of side effects, with nasal or oral administration, does not produce a satisfactory immune response without the use of an adjuvant. (Chen et al., 1989, Current Topics in Microbiology and Immunology146:101 106, Couch et al., 1997, J. Infect. Dis. 176:38 44). Thus, for example, for the optimum induction of the immune response with oral administration of an emulsion-inactivated vaccine, an antigen content between 66 μg antigen/dose and 384 μg antigen/dose is required (Avtushenko et al., 1996, J. Biotechnol. 44:21 28). Thus, this dose lies far above that of an inactivated vaccine for parenteral administration, which is at approximately 15 μg antigen/dose.

A cold-adapted, live attenuated influenza virus vaccine to be found in clinical studies for nasal administration is based on virus antigens from which reassortmants must be produced annually by means of genetic methods, in which the genes for the hemagglutinin and neuramidase antigens of the corresponding influenza A or B strain are transferred to an attenuated, cold-adapted master virus strain. This method is very time consuming and labor intensive. In addition, there is the danger that through reversion the attenuated virus back mutates into a virulent virus and thus can trigger viremia. When immunization is carried out with living viruses there is also a further spread in the body of the immunized individual. When cold-adapted viruses are used, there is also the constant necessity of storing the virus vaccine below the freezing point, as close to −20° C. as possible, which then requires the absolute maintenance of a chain of refrigeration to ensure sufficient storage life of the vaccine.

Eggs are used for the production of the live attenuated influenza virus vaccine virus reassortmants and the propagation of the vaccine viruses, which entails the risk that any contaminating infectious agents that may be present may be transferred into the eggs. The purification of live viruses is also not without problems because they represent infectious material and thus a higher standard of security must be maintained.

The availability of a technique for attenuating viruses in a rapid and non-random way would eliminate the disadvantages associated with current methods.

### SUMMARY

The invention relates to the development of modified viral polymerases that have an active site lysine residue substitution resulting in an altered rate of replication and altered fidelity compared to wild-type (WT) polymerases. In a typical embodiment, when incorporated into a virus, a modified polymerase as described herein exhibits a decreased rate of replication and a higher fidelity than a corresponding WT polymerase. In the experiments described herein, a poliovirus (PV) 3Dpol active site residue, Lys-359, was identified as a general acid catalyst during the phosphoryl transfer step of the nucleotide incorporation reaction. Surprisingly, subtle changes in polymerase speed and accuracy were found to have dramatic attenuation effects on the virus. Mutant PV viruses were made having a modified polymerase gene resulting in either a K359R or K359H substitution. These viruses were shown to be infectious and attenuated in cells relative to WT virus. The well-conserved residues in a polymerase active site as described herein modulate polymerase speed and/or accuracy, and are therefore targets for attenuating viruses. Active site lysine residues in polymerases from other classes were also identified and analyzed. Thus, the compositions and methods described herein can be applied to any virus, including viruses with regular intervals of antigenic shift (e.g., influenza), newly emerging and re-emerging viruses (e.g., SARS, West Nile, Dengue), and viruses used as agents of terror or biological weapons (e.g., Ebola, smallpox). Thus, the compositions and methods described herein provide a universal strategy for attenuating viruses for anti-viral vaccines. The present invention also solves the problem of generating vaccines of both highly virulent viruses and in amounts that induce either the humoral or cellular or both, immune responses.

Accordingly, the invention includes an attenuated virus including a polymerase gene that encodes a polymerase having a modification that results in a substitution of a lysine residue to a leucine, histidine, or arginine residue, the lysine residue capable of functioning as a general acid catalyst during a phosphoryl transfer step of a nucleotide incorporation reaction and located on helix O of A-family polymerases, helix P of B-family polymerases and on the loop of structural motif D of RNA-dependent RNA polymerases and reverse transcriptases. The substitution causes the polymerase to have increased fidelity compared to a polymerase encoded by a WT polymerase gene not having the modification. The polymerase can be, for example, a PV polymerase and the lysine residue at position 359 of an amino acid sequence encoded by a nucleic acid sequence having accession number V01148 (SEQ ID N0:9); an influenza polymerase and the lysine residue at position 481 of a sequence having accession number AAY44773 (SEQ ID NO:10); or an HIV-1 reverse transcriptase and the lysine residue at position 220 of a sequence having accession number 4139739 (SEQ ID NO:11). The polymerase can be, for example, an A-family polymerase, a B-family polymerase, or a DNA-dependent DNA polymerase. In a typical embodiment, the virus is infectious.

Also within the invention is a vaccine that includes (a) an attenuated virus including a polymerase gene that encodes a polymerase, the polymerase gene having a modification that results in a substitution of a lysine residue to a leucine, histidine, or arginine residue, the lysine residue capable of functioning as a general acid catalyst during a phosphoryl transfer step of a nucleotide incorporation reaction and located on helix O of A-family polymerases, helix P of B-family polymerases and on the loop of structural motif D of RNA-dependent RNA polymerases and reverse transcriptases, and a pharmaceutically acceptable carrier. The substitution causes the polymerase to have increased fidelity compared to a polymerase encoded by a WT polymerase gene not having the modification. The polymerase can be, for example, a PV polymerase and the lysine residue at position 359 of an amino acid sequence encoded by a nucleic acid sequence having accession number V01148; an influenza polymerase and the lysine residue at position 481 of a sequence having accession number AAY44773; or an HIV-1 reverse transcriptase and the lysine residue at position 220 of a sequence having accession number 4139739. The polymerase can be an A-family polymerase, a B-family polymerase, or a DNA-dependent DNA polymerase. The vaccine can further include an adjuvant.

In another embodiment, the invention includes a purified nucleic acid including a polymerase gene that encodes a polymerase, the polymerase gene having a modification that results in a substitution of a lysine residue to a leucine, histidine, or arginine residue, the lysine residue capable of functioning as a general acid catalyst during a phosphoryl transfer step of a nucleotide incorporation reaction and located on helix 0 of A-family polymerases, helix P of B-family polymerases and on the loop of structural motif D of RNA-dependent RNA polymerases and reverse transcriptases. The substitution causes the polymerase to have increased fidelity compared to a polymerase encoded by a WT polymerase gene not having the modification. The nucleic acid can be within a vector.

In still a further embodiment, the invention includes a method of attenuating a virus including modifying a viral polymerase gene that encodes a polymerase, such that the modification results in a substitution of a lysine residue to a leucine, histidine, or arginine residue, the lysine residue capable of functioning as a general acid catalyst during a phosphoryl transfer step of a nucleotide incorporation reaction and located on helix O of A-family polymerases, helix P of B-family polymerases and on the loop of structural motif D of RNA-dependent RNA polymerases and reverse transcriptases. The substitution causes the polymerase to have increased fidelity compared to a polymerase encoded by a WT polymerase gene not having the modification.

Unless otherwise defined, all technical terms used herein have the same meaning as commonly understood by one of ordinary skill in the art to which this invention belongs.

As used herein, the term “attenuated” means a virus that is modified to be less virulent (disease-causing) than WT virus.

As used herein, “vaccine” includes all prophylactic and therapeutic vaccines. According to one embodiment the vaccine contains an avirulent or attenuated infectious virus, the viral polymerase having amino acid changes in the conserved regions of the polymerase. These changes include for example, substituted amino acids, analogs, deletions and the like.

The vaccine compositions of the invention are suitable for administration to subjects in a biologically compatible form in vivo. The expression “biologically compatible form suitable for administration in vivo” as used herein means a form of the substance to be administered in which any toxic effects are outweighed by the therapeutic effects. The substances may be administered to any animal, preferably humans.

By the terms “RNA-dependent polymerase” and “RDP” is meant a viral polymerase that transcribes either RNA or DNA from an RNA template. RNA viruses described herein produce an RNA-dependent RNA polymerase (RDRP), and retroviruses described herein produce an RNA-dependent DNA polymerase, also referred to as a “reverse transcriptase.”

By the term “DNA-dependent DNA polymerase” is meant a viral polymerase that transcribes DNA from a DNA template.

As used herein, a “nucleic acid,” “nucleic acid molecule,” or “polynucleotide” means a chain of two or more nucleotides such as RNA (ribonucleic acid) and DNA (deoxyribonucleic acid). A “purified” nucleic acid molecule is one that has been substantially separated or isolated away from other nucleic acid sequences in a cell or organism in which the nucleic acid naturally occurs (e.g., 30, 40, 50, 60, 70, 80, 90, 95, 96, 97, 98, 99, 100% free of contaminants). The term includes, e.g., a recombinant nucleic acid molecule incorporated into a vector, a plasmid, a virus, or a genome of a prokaryote or eukaryote.

As used herein, “protein” or “polypeptide” are used synonymously to mean any peptide-linked chain of amino acids, regardless of length or post-translational modification, e.g., glycosylation or phosphorylation.

When referring to a nucleic acid molecule, polypeptide, or virus, the term “native” refers to a naturally-occurring (e.g., a WT) nucleic acid, polypeptide, or virus.

As used herein, the term “vector” refers to a nucleic acid molecule capable of transporting another nucleic acid to which it has been linked. Vectors capable of directing the expression of genes to which they are operatively linked are referred to herein as “expression vectors.”

A first sequence is “operably” linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein coding regions, in reading frame.

Although compositions and methods similar or equivalent to those described herein can be used in the practice or testing of the present invention, suitable compositions and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a pair of schematic illustrations of polymerase-catalyzed phosphoryl transfer. FIG. 1A depicts the two-metal-ion mechanism. The nucleoside triphosphate enters the active site with a divalent cation (Mg²⁺, metal B). This metal is coordinated by the β- and γ-phosphates of the nucleotide, by an Asp residue located in structural motif A of all polymerases, the 3Dpol residue is indicated, and likely water molecules (indicated as oxygen ligands to metal without specific designation). This metal orients the triphosphate in the active site and may contribute to charge neutralization during catalysis. Once the nucleotide is in place, the second divalent cation binds (Mg²⁺, metal A). Metal A is coordinated by the 3′-OH, the α-phosphate, as well as Asp residues of structural motifs A and C. This metal lowers the pKa of the 3′-OH (denoted as Hₐ) facilitating catalysis at physiological pH. FIG. 1B shows the proton transfer reactions. During the phosphoryl transfer reaction, two proton transfer reactions may occur. The proton from the 3′-OH nucleophile (Hₐ) must be removed; a proton may be donated to the pyrophosphate leaving group (H_{b}).

FIG. 2 is a pair of graphs showing two ionizable groups are required for phosphoryl transfer. Values for kₚₒₗ were obtained for AMP incorporation into S/S-+1 using the stopped-flow assay in Mg⁺² (FIG. 2A) or Mn⁺² (FIG. 2B). pH values greater than 10 in Mg⁺² and in Mn⁺² caused precipitation of nucleotide. The solid lines show the fit of the data to equation 3 for Mg⁺², yielding pKa values of 7.0±0.1 and 10.5±0.1 and to equation 4 for Mn⁺², yielding a pKa value of 8.2±0.1. The dashed line in FIG. 2B shows the predicted curve should yield an ionizable group with a pKₐ of 10.5 exist in Mn⁺².

FIG. 3 is a series of graphs showing solvent deuterium isotope effect on the kinetics of nucleotide incorporation in the pre-steady-state. Pre-steady-state incorporation of AMP into S/S-+1 in H₂O () or D₂O (▪) in Mg²⁺ (FIG. 3A) or Mn²⁺ (FIG. 3B). The solid line is the fit of the data to equation 1, yielding rate constants, k_{obs}, in Mg2+ of 30±4 s−1 and 10±1 s−1 for H₂O and D₂O, respectively; and of 10±1 s⁻¹ and 1.4±0.3 s⁻¹ in Mn²⁺. Steady state incorporation of AMP into S/S-+1 in H₂O () or D₂O (▪) in Mg²⁺ (FIG. 3C) or Mn²⁺ (FIG. 3D). The lines are the fit of the data to a line, yielding rates in Mg²⁺ of 3.4×10⁻⁴±1×10⁻⁵ μMs⁻¹ and 2.9×10^{-4±3×10}⁻⁵ μMs⁻¹ in H₂O and D₂O, respectively; and of 0.5±0.1 μMs⁻¹ and 0.3±0.1 μMs⁻¹ in Mn²⁺.

FIG. 4 is a pair of graphs showing that two protons are transferred during phosphoryl transfer. Proton inventory was performed in Mg²⁺ (FIG. 4A) or Mn²⁺ (FIG. 4B). kₙ is the observed rate constant for nucleotide incorporation at a particular mole fraction of D₂O. k_{H2O} is the observed rate constant for nucleotide incorporation in H₂O. n is the mole fraction of D₂O. The solid line represents the fit of the data to a two-proton-transfer model (equation 5). The dashed line indicates the predicted line from a one-proton-transfer model (equation 6).

FIG. 5 is a series of graphs showing that two protons are transferred during phosphodiester bond formation catalyzed by all polymerases. FIG. 5A shows the solvent deuterium isotope effect for other polymerases: [i] RB69 DdDp, [ii] T7 DdRp and [iii] HIV RT. Pre-steady-state rate constants for nucleotide incorporation were determined for all polymerases in H₂O () or D₂O (▪). The solid lines are the fits of the data to equation 1. The rate constants were: 160±15 s⁻¹ and60±6 s⁻¹ in H₂O and D₂O, respectively, for RB69 DdDp; 46±5 s⁻¹ and 8±1 s⁻¹ in H₂O and D₂O, respectively, for T7 DdRp; 140±14 s⁻¹ and 60±5 s⁻¹ in H₂O and D₂O, respectively, for HIV RT. FIG. 5B shows the proton inventory for other polymerases: [i] RB69 DdDp, [ii] T7 DdRp and [iii] HIV RT. The solid lines are the fit of the data to a two-proton-transfer model (equation 5). The dashed line indicates the predicted line for a one-proton-transfer model (equation 6). In all cases, a two proton-transfer model fit the data best.

FIG. 6 is a schematic illustration of a general base and a general acid in polymerase-catalyzed nucleotidyl transfer reactions. The data are consistent with a model in which activation of the nucleophile occurs with a pKₐ of 7.0 and protonation of the leaving group occurs with a pKₐ of 10.5. The plc of 7.0 is likely the 3′-OH as this pKₐ is modulated by the divalent cation employed and by the atom present at the α-position of the nucleotide substrate.

FIG. 7 illustrates 2-Aminopurine as a fluorescent probe for 3D^{pol}-catalyzed nucleotide incorporation reactions. FIG. 7A is a schematic illustration showing the 2-Aminopurine (2A) base which is fluorescent and has the capacity to form two basepairs with uracil, however, incorporation of UMP opposite 2A is never as efficient as incorporation opposite A. FIG. 7B shows a primer-template substrate used herein. It is a 10-nt self-complementary RNA, referred to as sym/sub. The templating positions have been designated as indicated: S/S-0, S/S-+1 and S/S-+2. SEQ ID NO:1 in the 5′ to 3′ direction is hybridized to SEQ ID NO:1 shown in the 3′ to 5′ direction. FIG. 7C is a series of graphs showing the magnitude and direction of the fluorescent transient depends on the location of 2A in the template. The actual sequences of the substrate employed for 2A at the S/S-0, S/S-+1 and S/S-+2 positions are indicated. Only the first “correct” nucleotide was evaluated: UTP (200 μM) for position 0 and ATP (200 μM) for positions +1 and +2. UMP incorporation into S/S-0 showed a 50% change in fluorescence and an observed rate constant, k_{obs}, of 1±0.1 s⁻¹. AMP incorporation into S/S-+1 showed a 25% change in fluorescence and a k_{obs} of 60±8 s⁻¹. AMP incorporation into S/S-+2 showed a 15% change in fluorescence and a k_{obs} of 50±6 s⁻¹. In the top graph, SEQ ID NO:13 in the 5′ to 3′ direction is hybridized to SEQ ID NO:13 shown in the 3′ to 5′ direction. In the middle graph, SEQ ID NO:14 in the 5′ to 3′ direction is hybridized to SEQ ID NO:14 shown in the 3′ to 5′ direction. In the bottom graph, SEQ ID NO:15 in the 5′ to 3′ direction is hybridized to SEQ ID NO:15 shown in the 3′ to 5′ direction.

FIG. 8 is a pair of graphs showing that stopped-flow fluorescence assay yields the same kinetic constants as the chemical-quench-flow assay. FIG. 8A shows the kinetics of AMP incorporation as a function of ATP concentration monitored by 2AP fluorescence. 3D^{pol} was incubated with S/S-+1 and mixed with 40-1200 μM ATP in a stopped-flow apparatus. The solid line represents the fit of the data to a single exponential equation (equation 1). FIG. 8B shows the values obtained in FIG. 8A were plotted as a function of ATP concentration. The solid line represents the fit of the data to a hyperbolic equation (equation 2) yielding values for K_{D,app} of 70±10 μM and kₚₒₗ of 70±10 s⁻¹.

FIG. 9 is a schematic representation showing a kinetic mechanism for 3D^{pol}-catalyzed nucleotide incorporation. The kinetic mechanism for the RNA dependent RNA polymerase (RdRp) from poliovirus (3D^{pol}) is shown as one embodiment. One of the advantages of this system is that once 3D^{pol} assembles onto the primer-template substrate, this complex has a half-life of greater than two hours, greatly simplifying kinetic analysis. In step one, the enzyme-nucleic acid complex (ERₙ) binds the nucleoside triphosphate forming a ternary complex (ERₙN). Step two involves a conformational change (*ERₙN) that orients the triphosphate for catalysis. In step three, phosphoryl transfer occurs (*ERₙ₊₁PPᵢ), followed by a second conformational-change step (ERₙ₊₁PPᵢ) and pyrophosphate release (ERₙ₊₁).

FIG. 10 is a schematic representation showing the structural motif D of PV polymerase is predicted in Flu polymerases. FIG. 10A shows the palm (catalytic) domain in PV polymerase depicted as cartoon model (pdb: 1RAJ) colored in grey with motif D highlighted in red. FIG. 10B shows motif D in PV polymerase with key residues Lys359 and Thr362 shown. FIG. 10C shows the modeled structure of motif D in influenza polymerase colored in cyan. Residues Lys481 and 11e484 at equivalent positions to Lys359 and Thr362 in B are displayed. FIG. 10D shows the alignment of residues in motif D of PB1 proteins from Influenza A, B and C viruses with poliovirus 3D^{pol}. Conserved residues are shown in red. In FIG. 10D, SEQ ID NOs: 16-19 are shown consecutively from the top down.

FIG. 11 illustrates sequence alignments and molecular modeling that reveal that PV 3Dpol RdRp Lys-359 is in position to function as a general acid catalyst. FIG. 11A: Amino acid sequence alignments of a variety of RNA polymerases show absolute conservation of a motif D active site lysine. PV, poliovirus; Cox, coxsackievirus; HRV14, human rhinovirus 14; RHDV, rabbit haemorrhagic disease virus; FCV, feline calicivirus; SARS, severe acute respiratory syndrome coronavirus; MHV, mouse hepatitis virus; HCV, hepatitis C virus; HIVRT, human immunodeficiency virus type-1 reverse transcriptase; QBeta, bacteriophage Q-beta. In FIG. 11A, SEQ ID NOs: 20-29 are shown consecutively from the top down. FIG. 11B: Location of Lys-359, Asp-328 and bound nucleotide (ATP) in model for poliovirus 3Dpol-ternary complex. Lys-359 interacts most closely with the β-phosphate of the nucleotide triphosphate moiety. FIG. 11C: Lys-359 is positioned to function as a general acid catalyst during phosphoryl transfer. FIG. 11Ci: Lys-359 is in spatial proximity to the β-phosphate prior to phosphoryl transfer. FIG. 11Cii: As the transition state of phosphoryl transfer is approached, the primer 3′-OH proton, Hₐ, is abstracted by an unidentified base and the c-amino group of Lys-359 donates its dissociable proton, H_{b}, to the non-bridging oxygen between the α- and β-phosphorus atoms.

FIG. 12 is a series of graphs showing pH rate profiles for PV 3Dpol WT and position 359 mutants support a role as general acid catalyst for Lys-359. FIG. 12A: pH-rate profile for WT K359 3Dpol shows that two ionizable groups influence the rate of phosphoryl transfer. Estimated pKas of the two groups are 7.0, believed to correspond to the 3′-OH proton, and 10.5, hypothesized to correspond to the Lys-359 proton. FIG. 12B: The basic arm of the pH-rate profile is lost in the 3Dpol K359L enzyme, indicating loss of one chemistry-influencing ionizable group. FIG. 12C: pH-rate profiles of K359H and K359R 3Dpols are superimposed over that for the K359L enzyme. The K359H polymerase is 5-fold kinetically superior to K359L at pH 7.5, approximately the pKa of the His imidazole group dissociable proton, but then converges toward K359L kinetic performance at higher pHs. The K359R polymerase is 5-fold kinetically superior to K359L at pH 7.5 but at higher pHs, as the pKa of the Arg side chain is approached, the catalytic ability of K359R approaches that of the WT K359 3Dpol.

FIG. 13 is an enlarged version of the graph shown in FIG. 12C.

FIG. 14 is a series of graphs showing proton inventories at pD 7.5 for WT K359 3Dpol and for K359L and K359R variants revealing that Lys-359 is the source of one proton transfer during catalysis. FIG. 14A: WT K359 proton inventory plot is bowl-shaped, indicating more than one rate-enhancing proton transfer during the transition state of phosphoryl transfer. The solid line is fit to a two-proton model (eq. 5) with a dashed straight line added as a visual reference. FIG. 14B: Proton inventory data for K359L 3Dpol are best fit with a one-proton, straight-line model (eq. 6) indicating that change of Lys-359 to chemically inert Leu resulted in loss of one rate-enhancing proton transfer during the phosphoryl transfer reaction. FIG. 14C: Proton inventory for K359R 3Dpol is bowl-shaped, indicating that more than one proton is transferred in the transition state when Arg occupies the 3Dpol 359 position.

FIG. 15 is a series of crystal structures for HIV-1, T7 and RB69 polymerases. A conserved active site Lys is in position to function as a general acid catalyst during phosphoryl transfer in polymerases from other classes. High-resolution x-ray crystal structures reveal the conserved active site lysine to be: FIG. 15A: Lys-220 of HIV-1 RT RdDp, FIG. 15B: Lys-631 of T7 DdRp and FIG. 15C. Lys-560 of RB69 DdDp. In each case the conserved Lys is believed to interact closely with the bridging oxygen between the α- and β-phosphorus atoms of the nucleotide triphosphate moiety.

FIG. 16 is a series of graphs showing proton inventories for WT polymerases and Leu mutants at the conserved active site Lys position, indicating a role as general acid catalyst for this Lys in all classes of nucleic acid polymerases. FIG. 16Ai: Proton inventory plot for HIV-1 RT WT Lys-220 RdDp is bowl-shaped indicating transfer of more than one proton in the transition state. Solid line is fit to a two-proton model (eq. 5) with dashed straight line added as a visual reference. FIG. 16Aii: Proton inventory for HIV-1 RT K220L RdDp is best fit with straight line model (eq. 6) indicating that replacement of Lys by chemically inert Leu at position 220 results in loss of one rate-enhancing proton transfer during phosphoryl transfer reaction. FIG. 16Bi: Similarly, WT RB69 Lys-560 DdDp proton inventory is bowl-shaped, indicating that more than one proton transfer enhances catalysis whereas Bii: proton inventory data for RB69 K560L are best fit by a straight line, indicating that only one proton transfer enhances catalysis in this variant. FIG. 16C: T7 DdRp WT Lys-631 proton inventory plot is bowl-shaped, indicating more than one proton transfer influencing the rate of the phosphoryl transfer step of nucleotide incorporation.

FIG. 17 is a graph illustrating that PV K359R,H subgenomic replicons replicate slower than the WT replicon.

FIG. 18 is a series of illustrations of polymerase structures and sequence alignments of polymerase sequences that show identification of the general acid in RNA-dependent RNA polymerases and reverse transcriptases. FIG. 18A (left panel): conserved structural motif D is a dynamic element that approaches the triphosphate moiety of the incoming nucleotide. Shown are ribbon representations of the superimposed RNA-dependent RNA polymerases (RdRps) from poliovirus (PV), foot-and-mouth disease virus (FMDV), Norwalk virus (NV) and human rhinovirus (HRV) types 14 and 16, as well as the RT from HIV1 (amino acid residues 1-315 of the catalytic p66 subunit). The displayed structures were taken from the following PDB files: PV-1RA6, 1RA7 (Thompson, A. A. & Peersen, O. B., Embo J. vol. 23:3462-3471, 2004)); FMDV-1U09, 1WNE, 2E9Z (Ferrer-Orta et al., J Biol Chem vol. 279:47212-47221, 2004; Ferrer-Orta et al., Proc Natl Acad Sci USA vol. 104:9463-9468, 2007); NV-1SH0, 1SH1, 3BSO (Ng et al., J Biol Chem vol. 279:16638-16645, 2004; Zamyatkin et al., J Biol Chem vol. 283:7705-7712, 2008)); HRV14:1XR5 (Love et al., Structure vol. 12:1533-1544, 2004); HRV16-1XR7(Love et al., Structure vol. 12:1533-1544, 2004); HRV1B-1XR6 (Love et al., Structure vol. 12:1533-1544, 2004); HIV1-RT-1RT1, 1RTD (Hopkins et al., J Med Chem vol. 39:1589-1600, 1996; Huang et al. Science vol. 282:1669-1775, 1998). The GTP substrate (from PV-binary complex structure) is shown as sticks at the catalytic cleft. FIG. 18A (right panel): a motif-D lysine approaches the triphosphate moiety of the incoming nucleotide. Shown is an enlargement of boxed area from FIG. 18A (left panel). This view clearly shows that Lys374 in NV RdRp (Lys359 in PV) approaches the triphosphate, although the distance and orientation are insufficient to support direct proton transfer. The dynamics of the conserved, structural element is even more obvious in this view. The inability to observe catalysis in the crystals of RdRp and RT complexes may suggest that motif D is not depicted in the active conformation or salvation state. FIG. 18B: only a single, conserved lysine residue exists in RdRps and RTs to serve as a general acid. The conserved lysine is labeled red. The motif D lysine of PV RdRp is Lys359 and of HIV RT is Lys220. Sequences for motifs D from the indicated RdRps and RTs were aligned based on structural superposition performed by using MUSTANG-0.3. The RdRp structures correspond to the following pdb codes: (PV, 1RA6; FMDV, 1U09; HRV14, 1XR5; HRV16, 1XR7; HRV1B, 1XR6; rabbit hemorrhagic disease virus (RHDV), 1KHW (Ng et al., J Biol Chem vol. 277:1381-1387, 2002); NV, 3BSO; hepatitis C virus (HCV), 1C2P (Lesburg et al., Nat Struct Biol vol. 6:937-943, 1999); bovine viral diarrhea virus (BVDV), 2CJQ (Choi et al., Structure vol. 14: 1107-1113, 2006); Dengue virus (DV), 2J7U (Yap et al., J Virol., vol. 81:4753-4765, 2007); West Nile virus (WNV), 2HCN (Malet et al., J Biol Chem vol. 282:10678-10689, 2007). The RT structures correspond to the following pdb codes: MuMLV, 1QAI (Najmudin et al., J Mol Biol. Vol. 296:613-632, 2000); HIV-1, 1RTD; HIV-2, 1MU2 (Ren et al., Proc Natl Acad Sci USA vol. 99:14410-14415, 2002); the catalytic subunit of telomerase TERT from Tribolium castaneum, 3DU5 (Gillis et al., Nature vol. 455:633-637, 2008). The gene used to produce the RdRp from HCV for structural studies had a Gln at the position of the general acid; however, this residue is a lysine in the infectious consensus clone (PubMed nucleotide accession number for the infectious clone=AJ238799). In FIG. 18B, SEQ ID NOs: 30-44 are shown consecutively from the top down. FIG. 18C: PV Motif D must move in order for Lys359 to serve as a general acid. Shown is a model of PV RdRp highlighting the location of motif D relative to the incoming nucleotide.

FIG. 19 is an illustration of a vaccination protocol and a pair of graphs showing percent survival in mice in a poliovirus challenge experiment. FIG. 19A is an illustration of the vaccination protocol used in the experiment described in FIGS. 19B and 19C. FIGS. 19B and 19C are a pair of graphs showing that K359R PV protects mice from a lethal challenge with WT PV. Five cPVR mice were immunized, intraperitoneal (IP), of 3D-K359R poliovirus. Four weeks after immunization, all animals were challenged IP with 5LD₅₀, 10⁸ PFU, of wild-type virus and monitored for signs of paralysis. Five cPVR mice were immunized IP with with 5×10⁵ PFU of 3D-K359R. Four weeks post-immunization, all animals were boosted with another dose of 5×10⁵ PFU of 3D-K359R. One week after boost, animals were challenged IP with 10⁸ PFU of wild-type virus and monitored for signs of paralysis.

### DETAILED DESCRIPTION

The invention encompasses compositions and methods relating to the development of modified viral polymerases that have an active site lysine residue substitution resulting in an altered rate of replication and altered fidelity compared to WT polymerases. The compositions and methods described herein provide a universal strategy for attenuating viruses for viral vaccines.

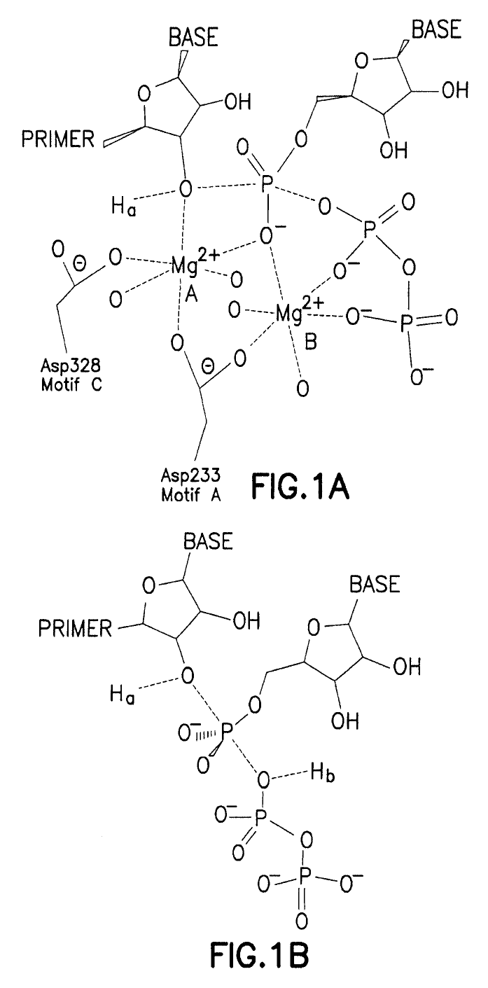

A two-metal ion mechanism has been proposed for polymerase-catalyzed nucleotidyl transfer reactions (Liu, J. & Tsai, M.-D. (2001) Biochemistry 40, 9014-9022; Steitz, T. A. (1993) Current Opinion in Structural Biology 3, 31-38). In this mechanism (FIG. 1A), metal A increases the nucleophilicity of the primer 3′-OH by lowering its pKₐ value, and metal B stabilizes the oxyanion that forms in the transition state and may facilitate pyrophosphate release. During nucleotidyl transfer, the proton from the 3′-OH of the primer (Hₐ in FIG. 1B) must be removed. Although it is not known whether the pyrophosphate leaving group must be protonated (H_{b} in FIG. 1B), pyrophosphate protonation should increase the rate of catalysis under physiological conditions given a pKa value in the 9.3 regime. Whether or not active-site residues function as acceptor or donor for these key proton transfer reactions was heretofore not known.

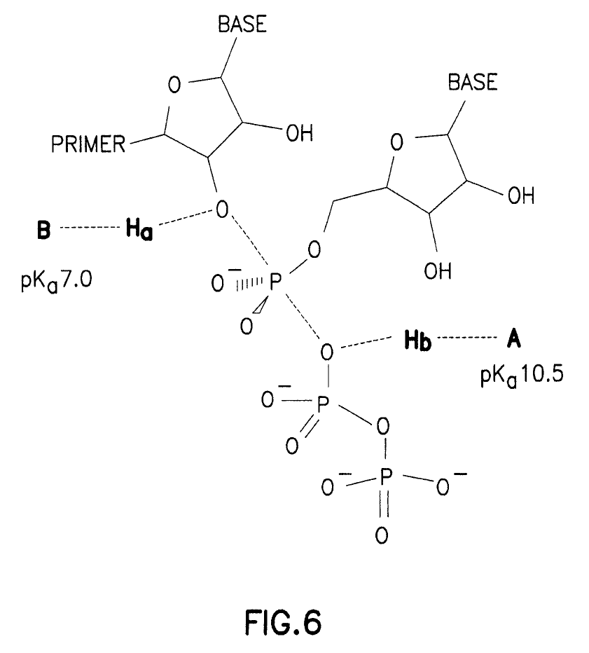

In the studies described herein, the mechanism of nucleotidyl transfer led to identification of a residue in the active site of all polymerases that facilitates nucleotide addition by protonating the pyrophosphate leaving group; this residue is Lys-359 in PV polymerase (FIG. 6). As shown in Table 1, substitution of different amino acids at this position of the polymerase yields enzymes with varying rates and fidelity of replication. Mutant PV viruses were made each having a modified polymerase gene resulting in either a K359R or K359H substitution. These viruses were shown to be infectious and attenuated in cells relative to WT virus. Because there is a functional equivalent of this residue in all viral polymerases, the capacity for one or more of the PV mutants to protect against challenge by WT PV will set the stage for establishment of a universally applicable, polymerase-mechanism-based strategy for production of virus vaccine strains.

TABLE 1
Mutation Frequency and replication rates for PV polymerase alleles

| | Mutation Frequency | Mutation Frequency | Replication | |
|---|---|---|---|---|
| Allele | sequencing¹ | kinetics² | Rate³ | |
| WT | 1.9 | 1/6,000 | 90+/5 | s⁻¹ |
| G64S | 0.5 | 1/8,600 | 30+/5 | s⁻¹ |
| K359L | nd⁴ | 1/450,000 | 0.50+/0.05 | s⁻¹ |
| K359H | nd | 1/13,500 | 5.0+/0.5 | s−1 |
| K359R | nd | 1/9,000 | 5.0+/0.5 | s⁻¹ |

¹The calculated average number of mutations per genome based upon sequencing 36,000 nucleotides of capsid coding sequence from 18 viral isolates.
²The calculated transition mutation frequency based upon the ratio of the kinetic parameters for correct and incorrect nucleotide incorporation by the PV RdRp allele.
³The maximal observed rate constant, kpol, for correct nucleotide incorporation.
⁴Not determined.

The below described preferred embodiments illustrate adaptations of these compositions and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Methods of propagating viruses for vaccine production and administering viral vaccines are also generally known in the art and are described in detail, for example, in Vaccine Protocols (Methods in Molecular Medicine) by Andrew Robinson, Martin P. Cranage, and Michael J. Hudson, 2^{nd} ed., Humana Press, Totowa, N.J., 2003; Vaccine Adjuvants and Delivery Systems, by Manmohan Singh, 1^{st} ed., Wiley-Interscience, Hoboken, N.J., 2007; Arvin A. M. and Greenberg H. B., Virology 344:240-249, 2006; and R. Morenweiser, Gene Therapy suppl. 1:S103-S110, 2005. Viral polymerase amino acid sequences and nucleic acid sequences encoding the viral polymerases are known in the art. For example, an HIV-1 RT amino acid sequence having accession number 4139739 is described in Huang et al. (Science 282:1669-1675, 1998). This HIV-1 RT is based upon a WT background, but has some modifications that allow for proper expression, crystallography and for trapping the enzyme in a covalent attachment with its RNA/DNA substrate. As another example, a human PV nucleic acid sequence having accession number V01148 has been described (Kitamura and Wimmer, Proc. Natl. Acad. Sci. 77:3196-3200, 1980). In yet another example, a human influenza A amino acid sequence having accession number AAY44773 has been described (Ghedin et al., Nature 437:1162-1166, 2005). These references are herein incorporated by reference.

### Attenuated Virus

The invention includes attenuated viruses having at least one mutation (e.g., substitution, deletion, insertion) at a conserved amino acid residue in the active site of the viral polymerase. Typically, such a modified polymerase includes a substitution of a lysine residue in the active site of the polymerase that is capable of functioning as a general acid catalyst during a phosphoryl transfer step of a nucleotide incorporation to a leucine, histidine, or arginine residue. This lysine residue as well as neighboring conserved amino acid residues are located on helix-O of A-family polymerases, helix P of B-family polymerases, and on a loop of motif D of RNA dependent RNA polymerases (RdRps) and reverse transcriptases (RTs).

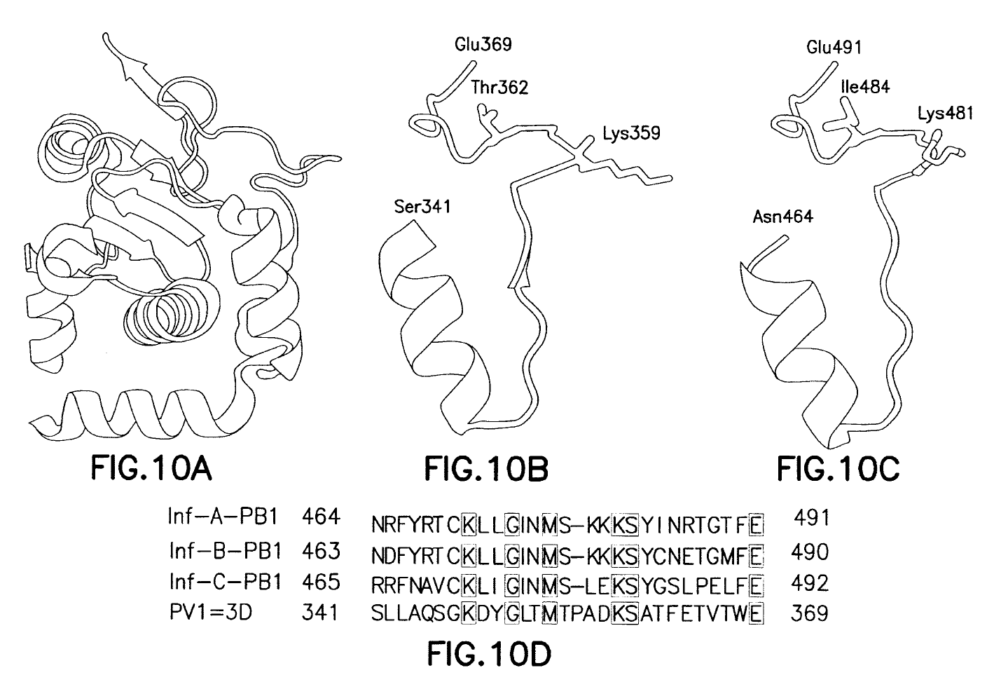

Viral RNA-dependent RNA polymerases have a conserved structure. Lys-359 of PV is present in the “palm” subdomain of the enzyme (FIG. 10A) on conserved structural motif D (FIG. 10B), a motif that has been modeled herein for flu (FIG. 10C). The structural homologue in flu to Lys-359 is Lys-481 of the PB1 subunit (FIG. 10C). This residue is found in influenza A, B and C genotypes (FIG. 10D). Together, these observations reinforce the notion that flu can be attenuated by using the polymerase-mechanism-based strategies described herein.

The comparable residue is located on helix O of A-family polymerases, helix P of B-family polymerases and on the loop of structural motif D of RdRps and RTs (Table 6). Many DNA virus polymerases are B-family polymerases, for example herpes viruses and poxviruses, and have this conserved Lys residue. These viruses can also be attenuated by using the polymerase-mechanism-based strategies described herein. In the experiments described below, PV mutant viruses K359R and K359H were found to replicate.

In some embodiments described herein, an attenuated virus is replication-deficient or non-replicating and includes a foreign nucleic acid that is expressed in a host cell. Examples of such nucleic acids include therapeutic molecules such as for example, cytokines, enzymes and the like.

Accordingly, the present invention also includes a method of preventing or treating a viral infection comprising administering a vaccine of the present invention to an animal in need thereof. For example, viral organisms which cause human diseases according to the present invention include (but are not restricted to) Filoviruses, Herpes viruses, Hepatitisviruses, Retroviruses, Orthomyxoviruses, Paramyxoviruses, Togaviruses, Picornaviruses, Papovaviruses and Gastroenteritisviruses.

In some embodiments, an attenuated virus is replication deficient or replication incompetent and is used in the manufacture of a vaccine. For example, fast replicating virulent viruses used in the preparation of vaccines, such as for example, influenza (e.g. H5N1), HIV, are deleterious to the host cells. An attenuated vaccine strain is advantageous in that the slower replication rate, and if desired, the virus is rendered non-replicating, prevents the virus strain from being lethal to the host due to a fast replication rate. Therefore, in a preferred embodiment, the replication rate is controlled by incorporating a mutation that changes the general acid from lysine to another residue (e.g., leucine, arginine, histidine). Generally, viral replication is decreased at least 24-fold compared to WT virus using the modified polymerases and methods described herein. In another embodiment, the attenuated virus strain is grown in host cells which are normally lysed by the normal virus. Examples include an attenuated HIV strain wherein the HIV strain is grown in human lymphocytes without the immediate lysis or death of these cells.

In one embodiment, a vaccine for a pathogenic virus includes an effective amount of attenuated virus for providing protection from challenge with the pathogenic virus and a pharmaceutically acceptable carrier. In this embodiment, the attenuated virus includes a polymerase gene that encodes a polymerase, the polymerase gene having a modification that results in a substitution of a lysine residue to a histidine or arginine residue, the lysine residue capable of functioning as a general acid catalyst during a phosphoryl transfer step of a nucleotide incorporation reaction and located on helix O of A-family polymerases, helix P of B-family polymerases and on the loop of structural motif D of RNA-dependent RNA polymerases and reverse transcriptases. This substitution causes the polymerase to have increased fidelity compared to a polymerase encoded by a wild-type polymerase gene not having the modification. For example, the pathogenic virus can be poliovirus and the polymerase a poliovirus polymerase, the lysine residue present at position 359 of an amino acid sequence encoded by the nucleic acid sequence of SEQ ID NO:9. In another example, the pathogenic virus can be an influenza virus and the polymerase an influenza polymerase, the lysine residue present at position 481 of an amino acid sequence encoded by the nucleic acid sequence of SEQ ID NO:10. In yet another example, the pathogenic virus can be HIV-1 and the polymerase an HIV-1 reverse transcriptase, the lysine residue present at position 220 of an amino acid sequence encoded by the nucleic acid sequence of SEQ ID NO:11. In such a vaccine, the polymerase can be an A-family polymerase, a B-family polymerase, a DNA-dependent DNA polymerase, an RNA-dependent RNA polymerase, or a reverse transcriptase. A vaccine as described herein can further include an adjuvant. A pathogenic virus is any virus that causes pathology in a subject or patient (e.g., a mammal such as a human). An effective amount of attenuated virus for providing protection from challenge with a pathogenic virus is an amount effective for preventing pathology caused by the pathogenic virus (e.g., paralysis caused by poliovirus, flu symptoms caused by influenza virus, acquired immune deficiency syndrome (AIDS) caused by HIV, etc.) in the subject. The attenuated virus is generally an attenuated version of the pathogenic virus (e.g., an attenuated poliovirus for protecting a subject from challenge with a naturally occurring or non-naturally occurring pathogenic poliovirus).

Described herein is a method of protecting a subject against a pathogenic virus including administering to the subject an effective amount of a vaccine for the pathogenic virus. The vaccine includes a pharmaceutically acceptable carrier and an attenuated virus including a polymerase gene that encodes a polymerase, the polymerase gene having a modification that results in a substitution of a lysine residue to a histidine or arginine residue, the lysine residue capable of functioning as a general acid catalyst during a phosphoryl transfer step of a nucleotide incorporation reaction and located on helix O of A-family polymerases, helix P of B-family polymerases and on the loop of structural motif D of RNA-dependent RNA polymerases and reverse transcriptases. The substitution causes the polymerase to have increased fidelity compared to a polymerase encoded by a wild-type polymerase gene not having the modification.

For example, the pathogenic virus can be poliovirus and the polymerase a poliovirus polymerase, the lysine residue present at position 359 of an amino acid sequence encoded by the nucleic acid sequence of SEQ ID NO:9. In another example, the pathogenic virus can be an influenza virus and the polymerase an influenza polymerase, the lysine residue present at position 481 of an amino acid sequence encoded by the nucleic acid sequence of SEQ ID NO:10. In yet another example, the pathogenic virus can be HIV-1 and the polymerase an HIV-1 reverse transcriptase, the lysine residue present at position 220 of an amino acid sequence encoded by the nucleic acid sequence of SEQ ID NO:11. In such a vaccine, the polymerase can be an A-family polymerase, a B-family polymerase, a DNA-dependent DNA polymerase, an RNA-dependent RNA polymerase, or a reverse transcriptase.

Also described herein is a method of inducing an immune response to a pathogenic virus antigen in a subject including administering to the subject an effective amount of a vaccine for the pathogenic virus wherein administration of the vaccine to the subject results in prevention of disease from the pathogenic virus. The vaccine includes a pharmaceutically acceptable carrier and an attenuated virus including a polymerase gene that encodes a polymerase, the polymerase gene having a modification that results in a substitution of a lysine residue to a histidine or arginine residue, the lysine residue capable of functioning as a general acid catalyst during a phosphoryl transfer step of a nucleotide incorporation reaction and located on helix O of A-family polymerases, helix P of B-family polymerases and on the loop of structural motif D of RNA-dependent RNA polymerases and reverse transcriptases. The substitution causes the polymerase to have increased fidelity compared to a polymerase encoded by a wild-type polymerase gene not having the modification. In one embodiment, the pathogenic virus is poliovirus and administration of the vaccine prevents paralysis in the subject. In other embodiments, the pathogenic virus is influenza or HIV. The vaccine can further include an adjuvant.

### Nucleic Acids

Modified viral polymerases as described herein are encoded by a viral polymerase gene that has a modification resulting in a mutation (e.g., substitution, deletion, insertion) at an amino acid residue in the active site of the polymerase that is capable of functioning as a general acid catalyst during a phosphoryl transfer step of a nucleotide incorporation reaction. One or more mutations in the conserved region of a viral polymerase modulate the activity of the polymerase (e.g., rate of replication, fidelity, etc.).

In the experiments described below, a PV polymerase was modified to include a substitution of leucine, arginine or histidine for the lysine corresponding to position 359 of the WT PV polymerase amino acid sequence encoded by a nucleic acid sequence having accession number V01148. Corresponding residues in HIV-1 and influenza were also identified. For example, the corresponding lysine in HIV-1 RT corresponds to position 220 of HIV-1 RT amino acid sequence having accession number 4139739. Similarly, the corresponding lysine in influenza corresponds to position 481 of the WT influenza gene segment PB1 amino acid sequence having accession number AA444773.

In some embodiments, a plurality of contiguous nucleic acids in the polymerase gene can be substituted with oligonucleotides which do not code for the conserved amino acid residues. Examples of some preferred oligonucleotides envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, —N(CH₃)—O—CH₂ [known as a methylene(methylimino) or MMI backbone], CH₂—O—N (CH₃)—CH₂, CH₂—N (CH₃)—N (CH₃)—CH₂ and O—N(CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also preferred. Also preferred are oligonucleotides having morpholino backbone structures (Summerton and Weller, U.S. Pat. No. 5,034,506). In other preferred embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). Oligonucleotides may also comprise one or more substituted sugar moieties. Preferred oligonucleotides comprise one of the following: OH, SH, SCH₃, F, OCN, OCH₃ OCH₃, OCH₃, O(CH₂)ₙCH₃, O(CH₂)ₙ NH₂ or O(CH₂)ₙ CH₃ where n is from 1 to about 10; C₁ to C₁₀ lower alkyl, alkoxyalkoxy, substituted lower alkyl, alkaryl or aralkyl; Cl; Br; CN; CF₃ ; OCF₃; O—, S—, or N-alkyl; O—, S—, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂ CH₂ OCH₃, also known as 2′-O-(2-methoxyethyl)] (Martin et al., Helv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me—C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl)adenine and 2,6-diaminopurine. Kornberg, A., DNA Replication, W. H. Freeman & Co., San Francisco, 1980, pp75-77; Gebeyehu, G., et al. Nucl. Acids Res. 1987, 15:4513). A “universal” base known in the art, e.g., inosine, may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are presently preferred base substitutions.

Another modification of the oligonucleotides of the invention involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). Oligonucleotides comprising lipophilic moieties, and methods for preparing such oligonucleotides are known in the art, for example, U.S. Pat. Nos. 5,138,045, 5,218,105 and 5,459,255.

It is not necessary for all positions in a given oligonucleotide to be uniformly modified, and in fact more than one of the aforementioned modifications may be incorporated in a single oligonucleotide or even at within a single nucleoside within an oligonucleotide. The present invention also includes oligonucleotides which are chimeric oligonucleotides as hereinbefore defined.

In another embodiment, a nucleic acid molecule of the present invention is conjugated with another moiety including but not limited to abasic nucleotides, polyether, polyamine, polyamides, peptides, carbohydrates, lipid, or polyhydrocarbon compounds. Those skilled in the art will recognize that these molecules can be linked to one or more of any nucleotides comprising the nucleic acid molecule at several positions on the sugar, base or phosphate group.

RNA Interference (RNAi): RNAi is a remarkably efficient process whereby double-stranded RNA (dsRNA, also referred to herein as siRNAs, for small interfering RNAs, or ds siRNAs, for double-stranded small interfering RNAs) induces the sequence-specific degradation of homologous mRNA in animals and plant cells (Hutvagner and Zamore, Curr. Opin. Genet. Dev., 12:225-232 (2002); Sharp, Genes Dev., 15:485-490 (2001)). In mammalian cells, RNAi can be triggered by duplexes of small interfering RNA (siRNA) (Chiu et al., Mol. Cell., 10:549-561 (2002); Elbashir et al., Nature, 411:494-498 (2001)), or by micro-RNAs (miRNA), functional small-hairpin RNA (shRNA), or other dsRNAs which are expressed in vivo using DNA templates with RNA polymerase III promoters (Zeng et al., Mol. Cell, 9:1327-1333 (2002); Paddison et al., Genes Dev., 16:948-958 (2002); Lee et al., Nature Biotechnol., 20:500-505 (2002); Paul et al., Nature Biotechnol., 20:505-508 (2002); Tuschl, T., Nature Biotechnol., 20:440-448 (2002); Yu et al., Proc. Natl. Acad. Sci. USA, 99(9):6047-6052 (2002); McManus et al., RNA, 8:842-850 (2002); Sui et al., Proc. Natl. Acad. Sci. USA, 99(6):5515-5520 (2002)).

The agents described herein can include dsRNA molecules that are targeted to (i.e., bind to) regions of residues conserved between polymerases and/or structural homologous regions between different polymerases. For example, lysine at position 359 of the poliovirus polymerase. (See, also, for example, Tables 1 and 6). Structurally homologous regions are readily identifiable using various programs, such as BLAST etc.

The dsRNA molecules typically include 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is identical or substantially identical to the first strand. Each strand can also have one or more overhanging (i.e., non-complementary) nucleotides, e.g., one, two, three, four or more overhanging nucleotides, e.g., dTdTdT.

The dsRNA molecules can be chemically synthesized, or can be transcribed in vitro from a DNA template, or in vivo from, e.g., shRNA. The dsRNA molecules can be designed using any method known in the art; a number of algorithms are known in the art, see, e.g., Tuschl et al., Genes Dev 13(24):3191-7 (1999), and many are available on the internet, e.g., on the websites of Dharmacon (Lafayette, Colo.) or Ambion (Austin, Tex.).

Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

micro RNA (miRNAs) of approximately 22 nucleotides can be used to regulate gene expression at the post transcriptional or translational level. miRNAs can be excised in the cell from an approximately 70 nucleotide precursor RNA stem-loop by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng (2002), supra). When expressed by DNA vectors containing polymerase III promoters, micro-RNA designed hairpins can silence gene expression (McManus (2002), supra).

dsRNA can be delivered directly into cells in vivo or in vitro using methods known in the art, e.g., cationic liposome transfection, nanoparticles, and electroporation, or expressed in vivo or in vitro from recombinant DNA constructs that allow longer-term target gene suppression in cells, including mammalian Pol III promoter systems (e.g., H1 or U6/snRNA promoter systems (Tuschl (2002), supra) capable of expressing functional double-stranded siRNAs; (Bagella et al., J. Cell. Physiol. 177:206-213 (1998); Lee et al. (2002), supra; Miyagishi et al. (2002), supra; Paul et al. (2002), supra; Yu et al. (2002), supra; Sui et al. (2002), supra).

Viral-mediated delivery mechanisms can also be used to induce specific silencing of targeted genes through expression of siRNA, for example, by generating recombinant adenoviruses harboring siRNA under RNA Pol II promoter transcription control (Xia et al. (2002), supra). Transcriptional termination by RNA Pol III occurs at runs of four consecutive T residues in the DNA template, providing a mechanism to end the siRNA transcript at a specific sequence. The dsRNA thus produced is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNAs when cotransfected into cells with a vector expression T7 RNA polymerase (Jacque (2002), supra).

In an animal, whole-embryo electroporation can efficiently deliver synthetic siRNA into post-implantation mouse embryos (Calegari et al., Proc. Natl. Acad. Sci. USA, 99(22):14236-40 (2002)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis, Nature Genetics 32:107-108 (2002)). Local delivery can also be used, e.g., with a carrier such as lipiodol (iodine in oil) to facilitate delivery into cells.

Engineered RNA precursors, introduced into cells or whole organisms as described herein, can be used for the production of a desired siRNA molecule. Such an siRNA molecule can then associate with endogenous protein components of the RNAi pathway to bind to and target a specific mRNA sequence for cleavage and destruction. In this fashion, the mRNA to be targeted by the siRNA generated from the engineered RNA precursor will be depleted from the cell or organism, leading to a decrease in the concentration of the protein encoded by that mRNA in the cell or organism. Additional information regarding the use of RNAi can be found in RNA Interference Editing, and Modification: Methods and Protocols (Methods in Molecular Biology), Gott, Ed. (Humana Press, 2004);

Antisense Polynucleotides: An “antisense” nucleic acid can include a nucleotide sequence that is complementary to a “sense” nucleic acid encoding regions of residues conserved between polymerases and/or structural homologous regions between different polymerases. For example, lysine at position 359 of the poliovirus polymerase. (See, also, for example, Tables 1 and 6).

Based upon the sequences disclosed herein, one of skill in the art can easily choose and synthesize any of a number of appropriate antisense molecules for use in accordance with the present invention. For example, a “gene walk” comprising a series of oligonucleotides of 15-30 nucleotides spanning the length of regions of residues conserved between polymerases and/or structural homologous regions between different polymerases. For example, lysine at position 359 of the poliovirus polymerase. (See, also, for example, Tables 1 and 6). The nucleic acid can be prepared, followed by testing for inhibition of viral replication. Optionally, gaps of 5-10 nucleotides can be left between the oligonucleotides to reduce the number of oligonucleotides synthesized and tested. Other methods, including computational analysis, RNAse H mapping, and antisense-oligonucleotide scanning microarrays, can also be used (see, e.g., DNA Microarrays: A Practical Approach, Schena, Ed. (Oxford University Press 1999; Scherr and Rossi, Nucl. Acids Res., 26(22):5079-5085 (1998)).

An antisense nucleic acid can be designed such that it is complementary to the entire coding region of a target polymerase but can also be an oligonucleotide that is antisense to only a portion of the coding or noncoding region of the target mRNA. For example, the antisense oligonucleotide can be complementary to a region surrounding the translation start site of the target mRNA, e.g., between the −10 and +10 regions of the target gene nucleotide sequence of interest. Preferably, the antisense nucleic acid is targeted to regions of residues conserved between polymerases and/or structural homologous regions between different polymerases. For example, lysine at position 359 of the poliovirus polymerase. (See, also, for example, Tables 1 and 6). An antisense oligonucleotide can be, for example, about 7, 10, 15, 20, 25, 30, 35, 40, 45, 50, 55, 60, 65, 70, 75, 80, or more nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest).

Antisense nucleic acid molecules are typically administered to a subject (e.g., by direct injection at a tissue site), or generated in situ such that they hybridize with or bind to mRNA and/or DNA encoding regions of residues conserved between polymerases and/or structural homologous regions between different polymerases. For example, lysine at position 359 of the poliovirus polymerase.

Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In yet another embodiment, an antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., Nucleic Acids. Res. 15:6625-6641 (1987)). The antisense nucleic acid molecule can also comprise a 2′-o-methylribonucleotide (Inoue et al. Nucleic Acids Res. 15:6131-6148 (1987)) or a chimeric RNA-DNA analogue (Inoue et al. FEBS Lett., 215:327-330 (1987)).

The potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Ribozymes: Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for regions of residues conserved between polymerases and/or structural homologous regions between different polymerases can include one or more sequences complementary to these nucleotide sequences and a sequence having known catalytic sequence responsible for mRNA cleavage (see U.S. Pat. No. 5,093,246 or Haselhoff and Gerlach, Nature, 334:585-591 (1988)).

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### Administration of Compositions

The vaccine compositions, attenuated viruses and compositions including attenuated viruses described herein can be introduced into a cell or administered to a subject (e.g., a human) in any suitable formulation by any suitable method. For example, the compositions and anti-viral vaccines described herein may be injected directly into a cell, such as by microinjection. As another example, the compositions and anti-viral vaccines described herein may be directly introduced into a subject (e.g., human), including by intravenous (IV) injection, intraperitoneal (IP) injection, subcutaneous injection, intramuscular injection, or in situ injection into target tissue. For example, a conventional syringe and needle can be used to inject a suspension containing an anti-viral vaccine as described herein into a subject. Depending on the desired route of administration, injection can be in situ (i.e., to a particular tissue or location on a tissue), IM, IV, IP, or by another parenteral route. Compositions and anti-viral vaccines as described herein can also be administered by oral administration, inhalation, transdermal administration, or suppository applications.

The vaccines of the present invention may additionally contain suitable diluents, adjuvants and/or carriers. Preferably, the vaccines contain an adjuvant which can enhance the immunogenicity of the vaccine in vivo. The adjuvant may be selected from many known adjuvants in the art including the lipid-A portion of gram negative bacteria endotoxin, trehalose dimycolate of mycobacteria, the phospholipid lysolecithin, dimethyldictadecyl ammonium bromide (DDA), certain linear polyoxypropylene-polyoxyethylene (POP-POE) block polymers, aluminum hydroxide, and liposomes. The vaccines may also include cytokines that are known to enhance the immune response including GM-CSF, IL-2, IL-12, TNF and IFNγ.

The dose of the vaccine may vary according to factors such as the disease state, age, sex, and weight of the individual, and the ability of antibody to elicit a desired response in the individual. Dosage regime may be adjusted to provide the optimum therapeutic response. For example, several divided doses may be administered daily or the dose may be proportionally reduced as indicated by the exigencies of the therapeutic situation. The dose of the vaccine may also be varied to provide optimum preventative dose response depending upon the circumstances.

### EXAMPLES

The present invention is further illustrated by the following specific examples. The examples are provided for illustration only and should not be construed as limiting the scope of the invention in any way.

### Example 1

### Two Proton Transfer Reactions in the Rate-Limiting Transition State for Nucleotidyl Transfer Catalyzed by RNA- and DNA-Dependent RNA and DNA Polymerases

### Materials and Methods

Materials: [γ-³²P]ATP (>7000 Ci/mmol) was purchased from ICN; [α-³²P] ATP (3000 Ci/mmol was from New England Nuclear; nucleoside 5′-triphosphates, (ultra pure solutions), were from Amersham Pharmacia Biotech, Inc; adenosine 5′-O-(1-thiotriphosphate) (ATPαS) was from Axxora Biochemicals (San Diego, Calif.); D₂O was purchased from Isotec (Miamisburg, Ohio); deuterated glycerol was from Cambridge Isotope Laboratories (Andover, Mass.); all RNA oligonucleotides were from Dharmacon Research, Inc. (Boulder, Colo.); all DNA oligonucleotides were from Integrated DNA Technologies (Coralville, Iowa); T4 polynucleotide kinase was from New England Biolabs, Inc; all other reagents were of the highest grade available from Sigma, Fisher, or VWR.

Abbreviations: DdRp, DNA-dependent RNA polymerase DdDp, DNA-dependent DNA polymerase; RNAp, DNA-dependent RNA polymerase; RT, reverse transcriptase; PV, poliovirus; S/S, symmetrical primer/template substrate; CQF, chemical quench flow instrument; SF, stopped-flow instrument; 2AP, 2-aminopurine; SDIE, solvent deuterium isotope effect; DTT, dithiothreitol; BME, beta-mercaptoethanol; EDTA, ethylenediamino tetra acetic acid; NP-40, nonident P-40; PMSF, phenylmethylsulfonyl fluoride; PEI, Polyethyleneimine.

### PV 3D^{mol}-Catalyzed Nucleotide Incorporation Experiments.

Stopped-flow experiments: Stopped-flow experiments were performed using a Model SF-2001 stopped-flow apparatus (Kintek Corp., Austin, Tex.) equipped with a water-bath. All reactions were performed at 30° C. Reactions were performed in two different sets of buffers. Reactions in HEPES refer to reactions performed by incubating 1 μM enzyme with 1 μM sym/sub (0.5 μM duplex) at room temperature for 3 minutes in 50 mM HEPES, pH 7.5, 10 mM 2-mercaptoethanol, 5 mM MgCl₂, 60 μM ZnCl₂, and then allowed to equilibrate to 30° C. in the sample compartment then rapidly mixed with the nucleotide prepared by using the same buffer. Reactions in MTCN or MHCN refer to reactions performed by incubating 1 μM enzyme with 1 μM sym/sub (0.5 μM duplex) in 1 mM HEPES pH 7.5 at room temperature for 3 minutes and then allowed to equilibrate to 30° C. in the sample compartment then rapidly mixed with the nucleoside triphosphate substrate in a solution containing 2×MTCN buffer at the indicated pH. 1×MTCN buffer is: 50 mM MES, 25 mM TRIS, 25 mM CAPS and 50 mM NaCl. The other buffer components for both enzyme-sym/sub and nucleotide solutions were the same. 1×MHCN buffer is: 50 mM MES, 25 mM HEPES, 25 mM CAPS and 50 mM NaCl. 3D^{pol} was diluted into enzyme buffer (50 mM HEPES, pH 7.5, 10 mM 2-mercaptoethanol, 60 μM ZnCl₂, and 20% glycerol) immediately prior to use. The volume of enzyme added to any reaction was always equal to one-twentieth the total reaction volume. For reactions with a final ATP concentration higher than 1 mM, the amount of free Mg²⁺ was kept constant by increasing the amount of MgCl₂ in the reaction to 5 mM plus the ATP concentration above 1 mM. For example, in a reaction containing 5 mM ATP, the final Mg²⁺ concentration was adjusted to 9 mM. The excitation wavelength used was 313 nm. Fluorescence emission was monitored by using a 370 nm cut-on filter (model E370LP, Chroma Technology Corp., Rockingham, Vt.). Reactions utilizing the nonhydrolyzable ATP analog α,β-methyleneadenosine 5′-triphosphate (AMPCPP) were done to investigate the possibility that observed changes in fluorescence resulted from alterations in the 2AP environment as a consequence of nucleotide binding. This proved not to be the case as changes in fluorescence were minimal over the time frame under investigation. The concentrations of nucleotide substrates and contents of buffers used are indicated in the appropriate figure and table legends. Rate constants for nucleotide incorporation were obtained by using the data analysis software of the instrument.

### Rapid Chemical-Quench-Flow Experiments.

Rapid mixing/quenching experiments were performed by using a Model RQF-3 chemical-quench-flow apparatus (KinTek Corp., Austin, Tex.). 3D^{pol}-sym/sub complexes and all buffer solutions were prepared in the same way as for the stopped-flow experiments described above. Reactions were quenched by addition of HCl to a final concentration of 1 M. Immediately after the addition of HCl, the solution was neutralized by addition of 1 M KOH and 300 mM Tris base (final concentration). The concentrations of nucleotide substrates and contents of buffers used are indicated in the appropriate figure and table legends.

### Rapid-Quench Product Analysis: Denaturing PAGE.

An equal volume of loading/quenching dye (85% formamide, 0.025% bromophenol blue, and 0.025% xylene cyanol, 90 mM EDTA) was added to 10 μl of the quenched reaction mixtures and heated to 70° C. for 2-5 min prior to loading 5 μl on a denaturing 23% polyacrylamide gel containing 1×TBE (2 mM EDTA, 89 mM boric acid, 87 mM Tris) and 7 M urea. Electrophoresis was performed in 1×TBE at 90 W. Gels were visualized by using a phosphorimager and quantified by using the ImageQuant software (Molecular Dynamics).

Steady-State Incorporation of AMP into Sym/Sub.

Steady-state experiments were performed by incubating 1 μM 3D^{pol} with 30 μM sym/sub (15 μM duplex) in 1 Mm HEPES buffer at pH 7.5 for 90 seconds at 30° C. then mixed with ATP in 2×MTCN buffer at pH 7.5. Reactions were quenched at various times by mixing with an equal volume of loading/quenching dye. Product analysis was performed by using denaturing PAGE as described above. All buffers and solutions were prepared in either H₂O or D₂O for each experiment. The pD was used instead of pH for the solutions in D₂O and was adjusted according to pD=pH+0.4.

### Proton Inventory.

Enzymes, substrates and buffers for the proton-inventory experiments were prepared in 100% water or 100% D₂O followed by mixing at the appropriate ratio to obtain 0, 25, 50, 75 or 100% D₂O. Deuterated glycerol was used in all solutions in D₂O. All data collection was performed in the stopped-flow instrument as indicated above. The pD was used instead of pH for the solutions in D₂O as described above.

### Data Analysis.

Time courses at fixed nucleotide concentrations were fit to the single exponential function:

[product]=A*exp(−k_{obs}*t)+C (1)

where A is the amplitude of the burst, k_{obs} is the observed first-order rate constant describing the burst, t is the time, and C is a constant. Time courses from the stopped-flow instrument were fit to the same equation using the instrument software. Data were fit by nonlinear regression using the program KaleidaGraph (Synergy Software, Reading, Pa.). The apparent binding constant (K_{D,app}) and maximal rate constant for nucleotide incorporation (kₚₒₗ) were determined using the equation:

k_{obs}=kₚₒₗ[NTP]/K_{D,app}+[NTP] (2)

The pKa values for the pH dependence of kₚₒₗ were obtained by fitting to a model describing two-ionizable groups (Bevilacqua, P. C. (2003) Biochemistry 42, 2259-65.):

kₚₒₗ=k_{ind}/(1+10̂(pKₐ₁−pH)+(pH−pKₐ₂)) (³)

or one-ionizable group (Bevilacqua, P. C. supra.):

kₚₒₗ=k_{ind}/(1+10̂(pKₐ−pH)) (4)

where k_{ind} is the pH-independent rate constant.

The proton-inventory data were fit to the modified Gross-Butler equation for a twoproton-transfer model (Schowen, R. L. & Venkatasubban, K. S. (1985) CRC Crytical reviews in Biochemistry 17, 1-44.):

kₙ/k_{H2O}=(1−n+n*φ₁)(1−n+n*φ₂) (5)

or for a one-proton-transfer model (Schowen et al):

kₙ/k_{H2O}=(1−n+n*φ) (6)

where kₙ is the observed rate constant at the different percentages of D₂O, k_{H2O} is the observed rate constant in water, n is the mole fraction of D₂O and φ is the inverse of the isotope effect for each ionizable group.

Equilibrium and rate constants reported were determined independently at least twice. Error was propagated through products and quotients by using equation 6:

Δz=((Δx/x)²+(Δy/y)²)̂½*z (7)

where x and y are the primary data, z is the product or quotient of x and y, and Δx, Δy and Δz are the corresponding error values (Aikens, D. A., Bailey, R. A., Moore, J. A., Giachino, G. G. & Tomkins, R. P. T. (1984) Principles and techniques for an integrated chemistry laboratory (Waveland Press, Prospect Heights, Ill.)).
Expression and purification of RB69 DdDp.

RB69 DdDp was expressed in BL21 (DE3) cells. Frozen cells were thawed and suspended in lysis buffer (100 mM KPO₄ pH 8.0, 20% glycerol, 20 mM DTT, 0.5 mM EDTA pH 8.0, 2.8 μg/mL pepstatin A, 2.0 μg/mL leupeptin) and lysed by passing through a French press at 1000 psi. Immediately after lysis, NP-40 was added to 0.1% (v/v) and PMSF was added to 2 mM. Polyethyleneimine (PEI) was added drop wise at 4° C. to a final concentration of 0.25% (v/v) and stirred for 30 min. This suspension was then centrifuged at 75,000×g for 30 min at 4° C. The supernatant was decanted and collected, and ground ammonium sulfate powder was slowly added to 60% saturation at 4° C. and stirred for 30 min. The ammonium sulfate precipitate was suspended in Buffer A (50 mM Tris pH 8.0, 20% glycerol, 10 mM DTT, 0.1% NP-40) and dialyzed overnight against 1 L Buffer A containing 75 mM NaCl using a 12-14,000 Da MWCO membrane. After dialysis, the sample was diluted in Buffer A to a final NaCl concentration of 50 mM. The sample was loaded onto a phosphocellulose column (approximately 1 mL bed volume/25 mg total protein) at a flow rate of 1 mL/min. The column was washed with ten column volumes of Buffer A containing 50 mM NaCl and the protein was eluted with Buffer A containing 150 mM NaCl. Fractions were pooled based upon their purity on 8% SDS-PAGE gels. The pooled fractions were diluted in Buffer A to a final salt concentration of 50 mM. The sample was loaded onto a Q-Sepharose column (1 mL bed volume/40 mg of protein) at 1 mL/min. The column was washed with ten column volumes of Buffer A containing 50 mM NaCl and the protein eluted with Buffer A containing 150 mM NaCl. Protein-containing fractions were pooled as before. The protein was concentrated using a small (0.5 mL) Q-Sepharose column. Loading and washing of the column was as described above. Elution was performed with Buffer A containing 500 mM NaCl.

### Expression and Purification of T7 DdRp.

Purification of T7 DdRp was done using the same protocol as for RB69 DdDp with the following modifications: 1. Ammonium sulfate was added to 40% saturation. 2. The phosphocellulose column was eluted using a linear gradient (6 column volumes) from 50 mM-700 mM NaCl in Buffer A. 3. The Q-Sepharose column was loaded and washed, and the protein eluted using a linear gradient (6 column volumes) from 50 mM-400 mM NaCl in Buffer A. Protein-containing fractions were pooled as before. Additional steps to concentrate the protein were not necessary.

Expression and purification of HIV RT.

HIV RT was expressed and purified as described previously (Le Grice, S. F. & Gruninger-Leitch, F. (1990) Eur J Biochem 187, 307-14).

### Solvent Deuterium Isotope Effect and Proton Inventory for RB69 DdDp.

Experiments were done in the stopped-flow instrument at 25° C. essentially as described (Yang, G., et al. (2002) Biochemistry 41, 2526-34.) with minor modifications. The assays were done in 1×MTCN pH 7.5, 10 mM MgCl₂, 1 mM dATP with 1 μM RB69 WT enzyme and 150 nM primer-template substrate. The DNA primer used was: 5′-CCGACCAGCCTTG-3′ (SEQ ID NO: 1); the DNA template used was: 5′-AAAGC(2AP)TCAAGGCTGGTCGG-3′ (SEQ ID NO: 2). The solvent deuterium isotope effect and proton inventory were performed as described above for 3D^{pol}.

### Solvent Deuterium Isotope Effect and Proton Inventory for T7 DdRp.

Experiments were done in the stopped-flow instrument at 30° C. essentially as described for 3D^{pol} with minor modifications. One μM RNA primer, 5′-UUUUGCCGCGCC-3′(SEQ ID NO: 3), was annealed to 1 μM DNA template, 5′-GGAATGC(2AP)TGGCGCGGC-3′ (SEQ ID NO: 4), and then incubated with 1.6 μM T7 WT for 10 minutes at room temperature in 1×MTCN pH 7.5, 10 mM BME, and 5 mM MgCl₂. The reaction was started by mixing the T7-primer-template complex with 3 mM ATP in MTCN pH 7.5, 10 mM BME, 7 mM MgCl₂, 100 mM NaCl (additional). The solvent deuterium isotope effect and proton inventory were performed as described above for 3D^{pol}.

### Solvent Deuterium Isotope Effect and Proton Inventory for HIV-RT

Pre steady state experiments were done in the chemical-quench-flow instrument at 37° C. essentially as described (Gotte, M., et al (2000) Journal of Virology, 3579-3585) with some modifications: 2 μM 32P-labeled DNA primer, 5′-TTAAAAGAAAAGGGGGGACTGGA -3′ (SEQ ID NO: 5), was annealed with 2.2 μM DNA template, 5′-CGTTGGGAGTGAATTAGCCCTTCCAGTCCCCCCTTTTCTTTTAAAAAGTGGCTAAGA-3′ (SEQ ID NO: 6), and incubated with 4 μM RT in a solution containing no metals. The reaction was started by addition of 200 μM dATP in reaction buffer containing 10 mM MgCl₂. After mixing, reactant concentrations were reduced by 50%. Reactions were performed by using a chemical quench flow instrument. Product analysis was performed essentially as described for PV 3D^{pol} with the following modifications. 1. Unlabeled DNA primer was added to the loading/quenching dye at a concentration 100-fold that of the labeled primer as described previously (Arnold, J. J., Ghosh, S. K., Bevilacqua, P. C. & Cameron, C. E. (1999) Biotechniques 27, 450-2, 454, 456). 2. A 15% polyacrylamide gel containing 40% formamide was used.

TABLE 2
Solvent deuterium isotope effect and two proton transfer reactions
are observed during nucleotidyl transfer by all polymerases.

| | D₂0 | Proton |
|---|---|---|
| Enzyme | Effect | inventory |
| PV RdRp | 3 ± 0.5 | 2 |
| RB69 DdDp | 4 ± 0.1 | 2 |
| T7 DdRp | 5 ± 1 | 2 |
| HIV RT | 2 ± 0.5 | In progress |

### Results

A rapid fluorescence assay for evaluation of 3D^{pol} catalyzed nucleotide incorporation was developed. This assay has been employed to evaluate the pH dependence of this reaction, providing evidence for two ionizable groups in the rate limiting step for nucleotide incorporation. Under physiological conditions, a solvent deuterium isotope effect was observed. A proton inventory experiment provided the first observation of a two-proton transfer reaction during polymerase-catalyzed nucleotide incorporation. Two proton-transfer reactions were also observed for nucleotidyl-transfer reactions catalyzed by the RB69 DNA-dependent DNA polymerase, T7 DNA-dependent RNA polymerase and HIV reverse transcriptase. Together, these data provide very compelling evidence for use of a general base and a general acid in polymerase-catalyzed nucleotidyl-transfer reactions.

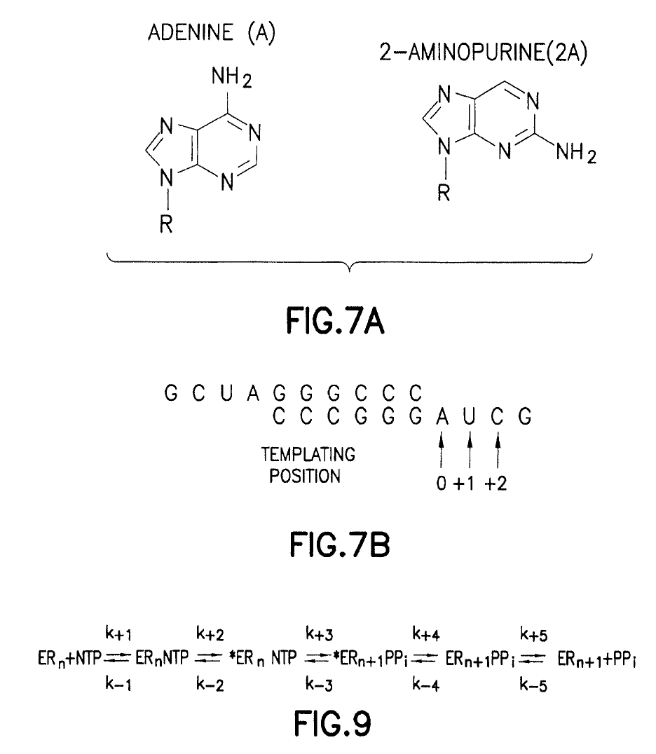

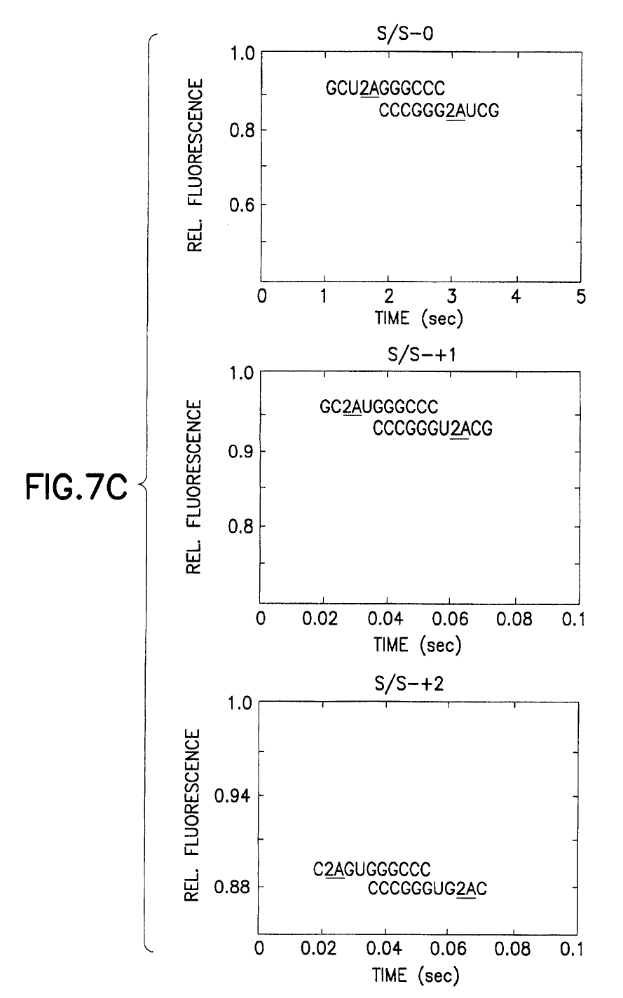

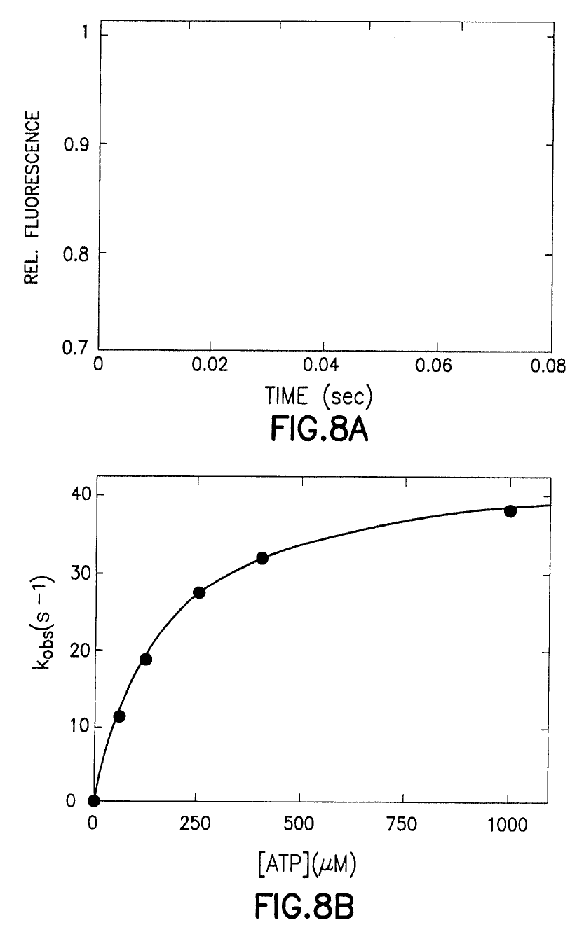

Two ionizable groups are required for RdRp-catalyzed nucleotidyl transfer. Because chemistry is at least partially rate limiting for 3Dpol-catalyzed nucleotide incorporation, we reasoned that it should be possible to obtain insight into the proton transfers occurring during the rate-limiting transition state by evaluating the pH dependence of the reaction. In order to maximize the amount of kinetic data obtained, we developed and validated a stopped-flow fluorescence assay for 3Dₚₒₗ that employed an RNA template containing 2-aminopurine (FIG. 7 and FIG. 8). In order to vary the pH of the reaction without varying the ionic strength, we chose the MTCN and MHCN buffer systems (Ellis, K. J. & Morrison, J. F. (1982) Methods Enzymol 87, 405-426). The buffers were comparable, yielding K_{D,app} and kₚₒₗ values of 200±20 μM and 50±10 s⁻¹, respectively, at pH 7.5 (Table 3). The higher K_{D,app} value for ATP relative to previous buffer systems employed was caused by the increased ionic strength of the MTCN and MHCN buffers.

TABLE 3
Effect of buffer composition on kinetic constants for 3D^{pol}-catalyzed
nucleotide incorporation^{a}.

| Buffer | Device | K_{D,app} (μM) | kₚₒₗ (s⁻¹) |
|---|---|---|---|
| HEPES | SF | 70 ± 10 | 70 ± 10 |
| HEPES | CQF | 70 ± 10 | 80 ± 10 |
| MTCN | SF | 200 ± 20 | 50 ± 10 |
| MHCN | SF | 200 ± 20 | 50 ± 10 |

^{a}Buffers are described in materials and methods. “SF” indicates that experiments were performed in a stopped flow instrument. “CQF: indicates that experiments were performed in a chemical-quench-flow instrument. Kinetic constants are reported to one significant figure.

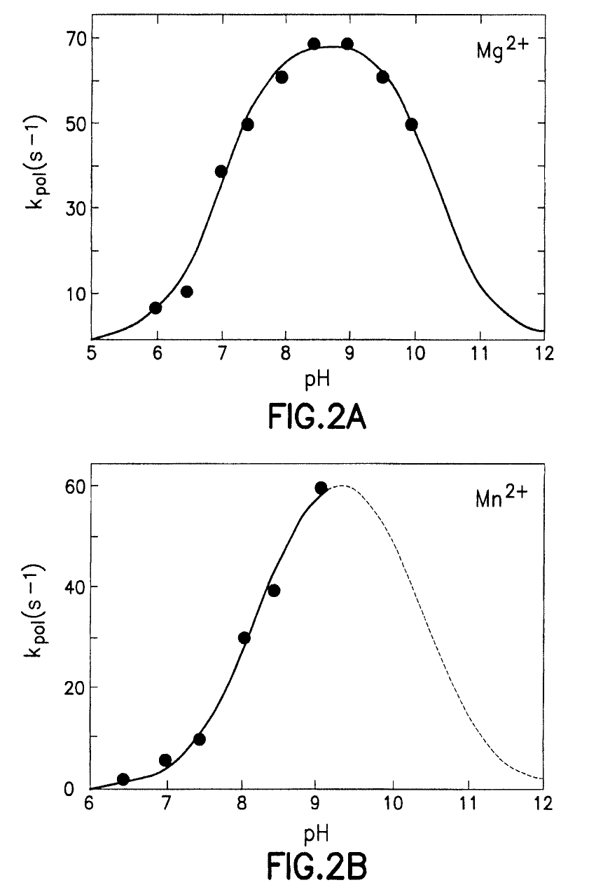

The K_{D,app} and kₚₒₗ values for nucleotide incorporation were measured at different pH values in Mg²⁺ or Mn²⁺ (Table 4). Experiments were limited to pH 10 in Mg²⁺ and pH 9 in Mn²⁺ due to nucleotide precipitation at higher pH values. Values for kₚₒₗ wereplotted as a function of pH (FIG. 2). In Mg²⁺, a bell-shaped curve was observed, indicative of two ionizable groups. The data were fit to equation 3, yielding pKₐ values of 7.1±0.1 and 10.5±0.1. In Mn²⁺, a similar pH dependence was observed for the acidic arm of the profile, but with a shift in pKₐ of about one pH unit to 8.2±0.1. These data fit well to a single ionization model. However, we could not rule out a two-ionization model in Mn²⁺. The pH rate profile that would be observed with pKₐ values of 8.2 and 10.5 is shown in FIG. 2B (dashed line). Data above pH 9 would be required to distinguish between the one and two-ionization models.

TABLE 4
Kinetic constants for AMP incorporation by PV 3Dpol in Mg²⁺ and Mn^{2+a}.

| Metal | | pH 6.0 | pH 6.5 | pH 7.0 | pH 7.5 | pH 8.0 | pH 9.0 | pH 9.5 | pH 10.0 |
|---|---|---|---|---|---|---|---|---|---|
| Mg²⁺ | K_{D,app}^{b} | 10 ± 1 | 30 ± 5 | 80 ± 10 | 200 ± 20 | 300 ± 30 | 500 ± 50 | 900 ± 70 | 1000 ± 100 |
| | kₚₒₗ^{b} | 7 ± 1 | 10 ± 1 | 40 ± 5 | 50 ± 5 | 60 ± 10 | 70 ± 10 | 60 ± 10 | 50 ± 10 |
| Mn²⁺ | K_{D,app} | | 2 ± 1 | 3 ± 1 | 5 ± 1 | 20 ± 2 | 20 ± 3 | | |
| | kₚₒₗ | | 3 ± 1 | 7 ± 1 | 10 ± 1 | 30 ± 5 | 60 ± 10 | | |

^{a}Experiments were performed as described in materials and methods.
^{b}Units for KD, app and kpol are μM and s⁻¹ respectively. Values are reported to one significant figure.

Rate limiting steps as a function of pH. In order to determine whether rate-limiting steps were changing as a function of pH, we evaluated the phosphorothioate (thio) effect over the pH range evaluated above (Table 5). For 3D^{pol}, chemistry is partially rate limiting in Mg²⁺ at pH 7.5. Under these conditions, the observed thio effect was 3±0.3 (Table 5). At pH values lower than 7.5, chemistry was clearly at least partially rate limiting as the value for the thio effect either did not change (pH 7.0) or increased (pH 6.0) (Table 5). A thio effect was also observed at pH values of 8.0 and 9.0; however, none was observed at pH 10 (Table 5). Interestingly, the decrease in the value of the thio effect above pH 7.5 was due to a specific increase in the observed rate constant for AMPαS incorporation without any significant effect on that for AMP incorporation (Table 5). In Mn²⁺ at pH 7.5, chemistry is the sole rate limiting step. From pH 6.0 to 9.0, the thio effect ranged from 7 to 5 (Table 5), consistent with chemistry remaining kinetically significant over this pH range.

TABLE 5
Phosphorothioate and solvent deuterium isotope effects on AMP
incorporation by PV 3Dpol-catalyzed nucleotide incorporation in
Mg⁺² and Mn+2. Values are observed rate constants,
AMP incorporation (in s⁻¹) at concentrations of
ATP 6-fold greater than the KD, app for ATP.

| | | | | | | |
|---|---|---|---|---|---|---|
| NTP/ | | | | | | |
| Solvent | pH 6.0 | pH 7.0 | pH 7.5 | pH 8.0 | pH 9.0 | pH 10.0 |

Reactions in Mg²⁺

| | | | | | | |
|---|---|---|---|---|---|---|
| ATP | 6 ± 1 | 30 ± 3 | 30 ± 3 | 40 ± 4 | 50 ± 10 | 40 ± 4 |
| ATP-αS | 1 ± 0.2 | 10 ± 1 | 10 ± 1 | 20 ± 2 | 30 ± 3 | 40 ± 4 |
| Effect | 6 ± 2 | 3 ± 0.4 | 3 ± 0.4 | 2 ± 0.3 | 2 ± 0.4 | 1 ± 0.1 |
| H₂O | 6 ± 1 | 30 ± 3 | 30 ± 4 | 40 ± 5 | 50 ± 10 | 40 ± 4 |
| D₂O | 1 ± 0.2 | 6 ± 1 | 10 ± 1 | 20 ± 2 | 20 ± 3 | 20 ± 2 |
| Effect | 6 ± 2 | 5 ± 1 | 3 ± 0.5 | 2 ± 0.3 | 3 ± 0.6 | 2 ± 0.3 |

Reactions in Mn²⁺

| | | | | | |
|---|---|---|---|---|---|
| ATP | 0.7 ± 0.1 | 4 ± 1 | 10 ± 1 | 20 ± 2 | 50 ± 10 |
| ATP-αS | 0.1 ± 0.05 | 0.6 ± 0.1 | 1.4 ± 0.3 | 3 ± 0.3 | 10 ± 2 |
| Effect | 7 ± 4 | 7 ± 2 | 7 ± 2 | 7 ± 1 | 5 ± 1 |

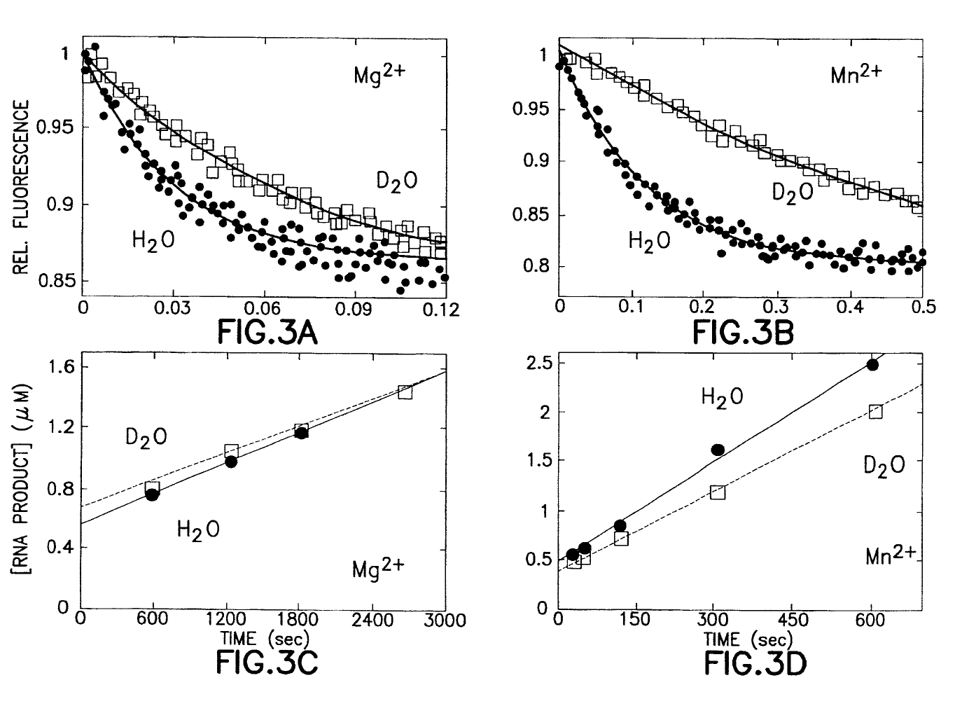

Solvent deuterium isotope effect as a probe for rate-limiting steps during nucleotide incorporation: Because it was difficult to interpret the loss of the thio effect in Mg²⁺ at pH 10.0, the solvent deuterium isotope effect was investigated. If proton transfers are occurring during the rate-limiting steps measured by the nucleotide incorporation assay, then an isotope effect should be apparent. In Mg²⁺ at pH 7.5, an isotope effect of 3±0.5 was observed for AMP incorporation in the pre-steady-state (FIG. 3A). In Mn²⁺ at pH 7.5, an isotope effect of 7±2 was observed (FIG. 3B). The dependence of the magnitude of the isotope effect on the divalent cation employed was consistent with previous observations that chemistry is partially rate limiting in Mg²⁺ but solely rate limiting in Mn²⁺. However, to rule out conformational perturbations as the cause of the observed isotope effects, AMP incorporation in the steady state was evaluated.

The rate-limiting step in the steady state is dissociation of enzyme from the primer template substrate. An isotope effect on this step would not be expected. As shown in FIGS. 3C and 3D, an isotope effect was not observed. It was concluded that the solvent deuterium isotope effect is a useful probe for chemistry as a rate-limiting step during nucleotide incorporation. Evaluation of the pH dependence of the solvent deuterium isotope effect in Mg²⁺ showed a constant value of 2±0.2 from pH 7.5 to 10.0 (Table 5). Therefore, chemistry is partially rate limiting over the entire pH range evaluated.

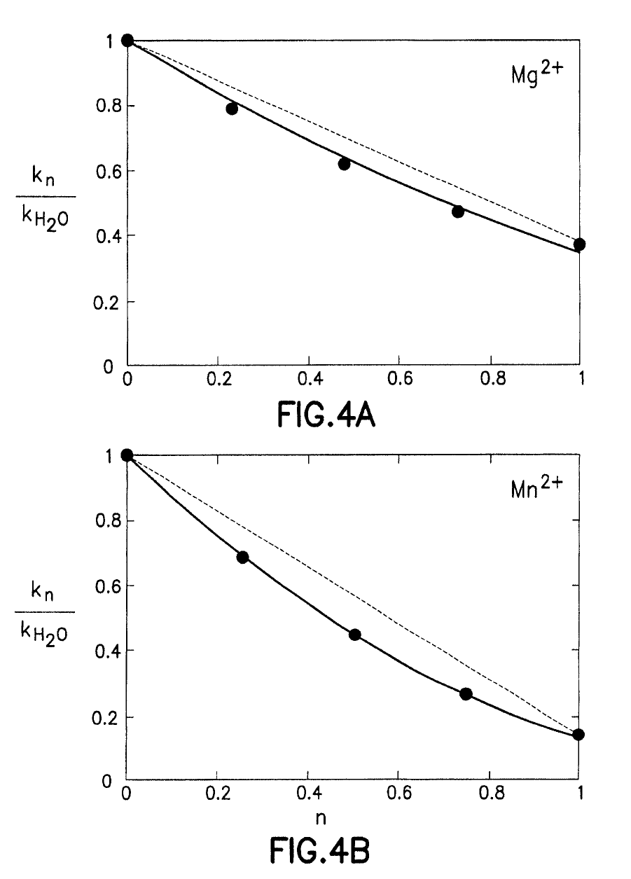

Two proton-transfer reactions in the rate-limiting transition state for RdRp catalyzed nucleotidyl transfer. The existence of a solvent deuterium isotope effect on the rate constant for nucleotide incorporation permits quantitation of the number of protons transferred in the rate-limiting transition state (proton inventory). In this experiment, the observed rate constant for nucleotide incorporation (kₙ) is measured in reactions containing different mole fractions of D₂O (n). A plot of the quotient kₙk_{H2O} (k_{H2O} is the observed rate constant in H₂O) as a function of n will fall on the line defined by k_{H2O}/k_{H2O} and k_{100%D2O}/k_{H2O} if a single proton is transferred. The data will fit to a second-order polynomial if two protons are transferred, to a third order polynomial if three protons are transferred and so on. A proton-inventory experiment was performed for AMP incorporation by 3D^{pol} in Mg²⁺ (FIG. 4A) and in Mn²⁺ (FIG. 4B). In neither case did the data fall on the line (dashed lines in FIGS. 4A, 4B) that would define a single proton transfer.

However, the data fit well to a second-order polynomial, the Gross-Butler equation (equation 5) for two-proton transfers (solid lines in FIGS. 4A, 4B). Based upon these data, we concluded that two proton transfer reactions occur in the rate-limiting transition state for phosphodiester bond formation catalyzed by the poliovirus RdRp.

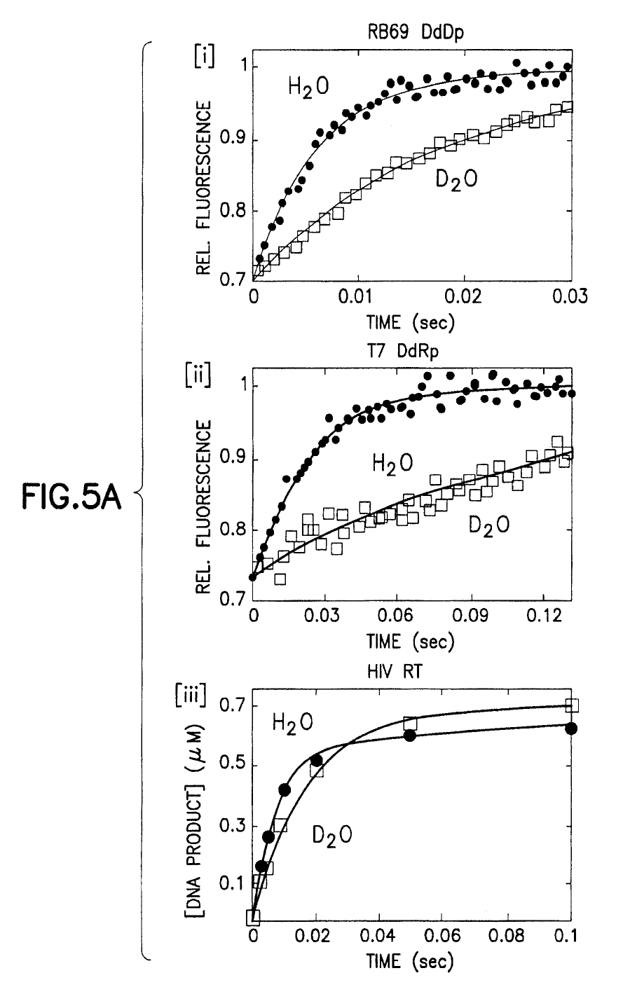

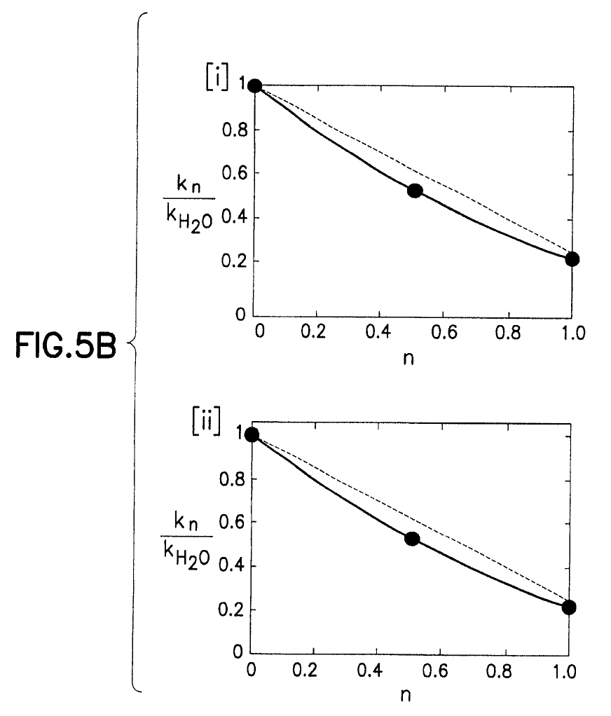

Two proton-transfer reactions in the rate-limiting transition state for nucleotidyl transfer of other classes of nucleic acid polymerases. In order to determine whether the conclusions reached here for the RdRp applied to other classes of nucleic acid polymerases, similar experiments were performed with RB69 DdDp, T7 DdRp and HIV RT. In all cases, a solvent deuterium isotope effect was observed (FIG. 5A, panels i-iii) that ranged from three to six (Table 1). Importantly, proton inventory experiments fit well to a two-proton model for these polymerases (FIG. 5B, panel i-iii and Table 2). It was concluded that two proton transfer reactions occur in the rate limiting transition state for nucleotidyl transfer catalyzed by all classes of nucleic acid polymerases.

3Dₚₒₗ-catalyzed nucleotide incorporation can be monitored by using 2-aminopurine containing primer/template substrates. Previous studies of 3D^{pol} mechanism revealed that the rate of nucleotide incorporation catalyzed by 3D^{pol} is partially limited by both a conformational change and chemistry in the presence of Mg²⁺. However, in the presence of Mn²⁺, the reaction is limited solely by chemistry. The experiments leading to these conclusions employed radioactively labeled substrates and a chemical quench flow instrument (CQF). This approach is slow and provides no direct information on conformational changes that may occur during the reaction. 2-Aminopurine (2AP) has been employed to study DNA-dependent DNA and RNA polymerase-catalyzed reactions. 2AP is similar in structure to adenine, which has its amino group on the 6-position (FIG. 7A). 2AP can replace adenine in a nucleic acid duplex without significant alteration of the helical structure. We investigated the use of 2AP as a fluorescent probe for single-nucleotide addition when present at various positions in the templating strand of our class of primer-template substrates referred to as sym/sub (S/S) (FIG. 7B). Position 0 indicates that 2AP is the templating nucleotide (S/S-0). Position+1 indicates 2AP is one nt downstream of the templating nucleotide (S/S-+1), and position +2 indicates 2AP is two nts downstream of the templating nucleotide (S/S-+2). The magnitude and direction of the observed change in 2AP fluorescence (AF) was dependent on the position of 2AP relative to the templating nucleotide (FIG. 7C). Incorporation of UMP into S/S-0 (FIG. 7C) produced the largest ΔF and signal-to-noise ratio. However, this substrate has the undesirable feature that the incoming nucleotide is not templated by a “correct” base. In the case of 2AP, incorporation of UMP is slower than expected (1±0.1_{S-1}), but on par with that observed in the chemical quench flow. As 2AP was moved downstream of the templating base, the ΔF and signal-to-noise ratio decreased, but the observed rate constants for incorporation (60±8_{S-1} and 50±6_{S-1} for S/S-+1 and S/S-+2, respectively) were as expected for AMP incorporation opposite UMP. The amplitude and signal-to-noise were better for 2AP in the +1 position than in the +2 position. All subsequent SF experiments utilized S/S-+1.

Validation of the SF assay. We previously determined the complete kinetic mechanism of 3D^{pol}-catalyzed nucleotide incorporation using radiolabeled S/S in the CQF. The values obtained in those experiments for K_{D,app} and kₚₒₗ were 130±20 μM and 90±10 s⁻¹, respectively. Use of S/S-+1 in CQF experiments produced values for K_{D,app} and kₚₒₗ of 70±10 μM and 80±10_{S-1}, respectively (Table 3). We interpret the decrease in K_{D,app} as a nearest neighbor effect caused by the 2AP. Note that the kₚₒₗ values are the same—that is, within the error of the measurement.

Experiments with S/S-+1 in the SF were performed with an excitation wavelength of 313 nm, ensuring excitation of only 2AP and guaranteeing that the observed changes in fluorescence were attributable only to changes in the environment surrounding the 2AP probe in the active site of 3Dpol. The observed change in fluorescence was measured as a function of ATP concentration (FIG. 8A). The observed rate constant, k_{obs}, at each ATP concentration was plotted as a function of ATP concentration and the data were fit to a hyperbola (equation 2), yielding values for K_{D,app} and kₚₒₗ of 70±10 μM and 70±10 s−1, respectively (FIG. 8B). It was concluded that the 2AP assay reports on product formation or some faster step thereafter.

Interrogation of the chemical mechanism for nucleotidyl transfer catalyzed by DNA polymerases has been thwarted by the general belief that a conformational change is rate limiting for nucleotide incorporation, a belief that has relied on interpreting the magnitude of the thio effect, a highly controversial parameter (Joyce, C. M. et al. (2004) Biochemistry. 43, 14317-24; Showalter, A. K. et al. (2002) Biochemistry 41, 10571-6). The studies described herein of the RdRp from poliovirus, however, have shown that chemistry is at least partially rate limiting for nucleotide incorporation in Mg²⁺ and completely rate limiting in Mn²⁺. This system permits interrogation of the chemical mechanism by employing a nucleotide incorporation assay.

Evaluation of the pH dependence of PV polymerase-catalyzed nucleotide incorporation in Mg²⁺ revealed a dependence of the reaction on two ionizable groups with pKa values of 7.0 and 10.5 (FIG. 2A). The pKa value of 7.0 was assigned to the 3′-OH because protonation of this group reduces the efficiency of nucleotide incorporation and chemistry is clearly rate limiting below pH 7.5 (Table 5). The pKa value of 10.5 was assigned to a residue on the enzyme that serves to protonate the PPi leaving group because deprotonation of this group reduces the efficiency of nucleotide incorporation and the extent to which chemistry is rate limiting in the pH range from 7.5 to 10.5 is the same (Table 5).

The observed pKa value for the 3′-OH is sensitive to the disposition of residues in the active site as distortions caused by increasing the size of the divalent cation or substituting ATPαS for ATP cause an increase in the observed pKa value (FIG. 2B and Table 5). The observed reduction in the nucleophilicity of the 3′-OH when ATPαS is employed provides an explanation for the observed reduction in the thio effect at pH values above 7.5. This observation calls into question the validity of the thio effect in polymerase systems. In order for the magnitude of the thio effect to yield information on rate-limiting steps during the nucleotide-addition cycle, the observed reduction in the rate constant for AMPaS incorporation relative to AMP incorporation should be attributable solely to the reduced electrophilicity of the α-phosphorous caused by the sulfur substitution. Extrapolating the RdRp data to other polymerase systems, the observed rate constant for AMP incorporation should not be compared directly to the observed rate constant for AMPaS incorporation at the same pH value.

The solvent deuterium isotope effect is a useful probe for chemistry in the rate limiting step for nucleotide incorporation by the RdRp (FIG. 3 and Table 5). The observed values for the solvent deuterium isotope effect ranged from two in Mg²⁺ at pH 7.5 (FIG. 3A) where chemistry is only partially rate limiting (1) to seven in Mn²⁺ at pH 7.5 (FIG. 3B) where chemistry is completely rate limiting. No significant isotope effect was measured in the steady state in the presence of either divalent cation (FIGS. 3C and 3D), consistent with the rate constant for polymerase dissociation being measured under these conditions. Application of the solvent deuterium isotope effect to other polymerase systems (FIG. 5A) revealed chemistry as partially rate limiting for nucleotide incorporation catalyzed by RB69 DdDp, T7 DdRp and HIV RT and validated the solvent deuterium isotope effect as a useful mechanistic probe of rate-limiting steps for all polymerases (Table 2).

The observation of a solvent deuterium isotope effect permitted the quantification of the number of proton-transfer reactions occurring during the rate-limiting transition state for nucleotide incorporation catalyzed by the RdRp (FIG. 4) and other polymerases as well (FIG. 5B). In all cases, two proton-transfer reactions were observed (Table 2). This observation would suggest that conversion of the 3′-OH to the 3′-O-does not occur as a discrete step prior to attack of the α-phosphorous of the bound nucleotide and provides the first evidence that the PP, leaving group does indeed leave protonated.

A solvent deuterium isotope effect of seven was observed for the RdRp under conditions in which chemistry is the sole rate-limiting step for nucleotide incorporation (FIG. 3), consistent with both protons being transferred simultaneously in the rate-limiting transition state. Efficient proton transfer would necessitate a suitable proton acceptor and donor. As discussed above, structural studies have not identified water molecules to serve this function. Moreover, the pKa values of 7.0 and 10.5 (FIG. 2) observed here for PV polymerase-catalyzed nucleotidyl transfer are also inconsistent with water serving as the proton acceptor or donor. The data described herein suggest that a general base and a general acid are employed in nucleotidyl-transfer reactions catalyzed by all polymerases (FIG. 6). There is a general consensus that the structural homologue of polβ Asp-256 in other systems is the evolutionarily conserved Asp residue of motif C (Table 6). In many polymerase structures, this motif C residue is often found serving as a ligand for one or both metals. This circumstance permits distance arguments to be used against this residue serving as a general base. However, in structures of a Bacillus stearothermophilus DNA polymerase I fragment (BF) undergoing catalysis, this motif C residue interacts with the primer 3′-OH after nucleotide binding but prior to binding of the second metal ion (metal A). In addition, computational modeling of the nucleotidyl transfer reaction catalyzed by the T7 DdDp has suggested that the conserved motif C residue, Asp-654, serves as a general base.

It is generally assumed that the pKₐ value for the Mg²⁺-bound PPi is low enough to preclude protonation during catalysis. However, in solution the highest pKₐ value for PPi is on the order of 9.3. Therefore, protonation of PPi by a general acid should increase the rate of catalysis but may not be absolutely essential. Structural studies have revealed a basic amino acid, in most cases a lysine, in a position to serve as a general acid (Table 6). This residue is located on helix O of A-family polymerases, helix P of B-family polymerases and on the loop of structural motif D of RdRps and RTs. Motif D has been in search of a function other than structural scaffolding since solution of the first RT structures. In all cases in which this putative general acid has been changed, the observed rate constant for nucleotide incorporation has been diminished substantially. Particularly noteworthy is the observation that chemistry appears to become the rate-limiting step by changing the putative general acid of RB69 DNA polymerase, Lys-560, to alanine.

The RdRp from PV was used as a model in the studies described herein to produce the first comprehensive analysis of the chemical mechanism for a nucleic acid polymerase. This system has proven to be particularly attractive because of the extremely slow (0.0001 s⁻¹) and pH-independent nature of the rate constant for dissociation of the primer-template substrate from the enzyme. These studies have provided very compelling evidence for a highly symmetrical transition state with proton transfers from the 3′-OH and to PPi occurring coordinately. Pyrophosphate protonation was not expected. All classes of polymerase appear to have a similar transition-state structure. Coordinated proton transfer reactions necessitate a well-positioned acceptor and donor. The observed pKₐ values required for nucleotidyl transfer and the absence of ordered water molecules in the appropriate position in all cases force one to re-propose general-acid-base-facilitated catalysis by all polymerases.

TABLE 6
Putative general acid and general base residues
for nucleic acid polymerases

| | DNA | PDB | | Motif C^{a} | Putative |
|---|---|---|---|---|---|
| | Pol | accession | | (general | general |
| Enzyme | Family | number | Motif A | base) | acid |
| T7 DdDP | A | 1t7p | D475 | D654, E655 | K522 |
| E. coli DNA Pol | A | | D705 | D882, E883 | K578 |
| I DdDP (KF) | | | | | |
| Bacillus stear. | A | 4bdp | D653 | D830, E831 | K706 |
| DNA Pol I | | | | | |
| DdDP (BF) | | | | | |
| Therm. aquat. | A | 3ktq | D610 | D785, E786 | K663 |
| DNA Pol I | | | | | |
| DdDP (Klentaq) | | | | | |
| RB69 DdDP | B | lig9 | D411 | D621, D623 | K560 |
| Human Pol β | X | 2fms | D190, | D256 | R183 |
| DdDP | | | D192 | | |
| HIV-1 RT RdDP | RT | 1rtd | D110 | D185, D186 | K220 |
| T7 DdRP | — | 1msw | D537 | D812 | K631 |
| PV 3D Pol | — | 1ra7 | D233 | D328, D329 | K359 |
| RdRP | | | | | |

^{a}Putative general base indicated in bold-faced type.

### Example 2

### General Acid Catalysis is Important in the Phosphoryl Transfer Reaction of Nucleic Acid Polymerases

The experiments described herein show that poliovirus RNA-dependent RNA polymerase (3Dpol) active site Lys-359 enhances the rate of phosphoryl transfer by acting as a catalytic general acid. This finding was extended to conserved active site lysine residues in polymerases from other classes by the demonstration of a role as catalytic general acid for the conserved active site lysine of HIV-1 RT RdDp and RB69 DdDp. General acid catalysis by a conserved active site residue capable of proton donation may therefore be a universal feature of the nucleic acid polymerase phosphoryl transfer reaction, adding an additional 2-3 orders of magnitude of rate enhancement to that already supplied by the two metal cations, and bringing rates of nucleotide incorporation to a level of biological sufficiency.

Nucleic acid polymerases are the primary agents of genome replication, maintenance and expression in all organisms and viruses. To fulfill this role, these enzymes must not only assemble nucleic acid chains at biologically sufficient rates but must also achieve a level of accuracy of nucleotide selection (fidelity) that is optimal for fitness.

Nucleic acid polymerases vary widely in their nucleic acid tasks. They require DNA or RNA as template. In most situations, they add nucleotides to a DNA, RNA or peptide primer-terminus 3′-OH. However, in some cases a primer-terminus requirement is lacking and nucleotide addition occurs de novo. Polymerase fidelity likewise varies widely. Misincorporation frequencies range from as low as 10⁻⁸ errors per incorporation in some genome-replicating DNA polymerases, to 10⁻⁵ for certain viral RNA polymerases to 10⁻² or higher for some repair enzymes. In all cases, misincorporation rates are thought to reflect biological need. Enzyme size and architectural complexity also vary widely, ranging from small, single-subunit enzymes having only polymerase activity to large, multi-subunit macromolecule assemblages possessing additional activities.

The broad range in biological roles, incorporation accuracies and overall architecture thus impose multiple functional constraints on the polymerase nucleotide incorporation mechanism. Yet in spite of this complexity, the most basic underlying polymerase active site structural features are highly conserved. In addition, all nucleic acid polymerases accomplish the same fundamental chemical reaction of phosphoryl transfer and use essentially the same five-step mechanism in the single nucleotide incorporation cycle. In step one an incoming nucleotide binds to the enzyme-primer/template binary complex. In step two this ternary complex undergoes a conformational change, aligning reactive functional groups for phosphoryl transfer, which occurs in step three. A post-chemistry conformational change occurs in step four. Pyrophosphate release occurs in step five, resetting the cycle to start the next incorporation.

Polymerase amino acid sequence alignments in combination with a growing list of high-resolution x-ray crystal structures of polymerase-primer/template-nucleotide ternary complexes reveal the universal presence of an active site positively charged amino acid residue, usually Lys but sometimes Arg or His, in the precise location of covalent bond cleavage and leaving group formation during step three, phosphoryl transfer, of the nucleotide incorporation cycle. Conservation of a residue capable of proton donation at this specific location suggests a direct role as general acid catalyst during phosphoryl transfer for this active site amino acid. However, a long-standing experimental limitation of some well-studied nucleic acid polymerase model systems has been the inability to interrogate the phosphoryl transfer step directly and conveniently because of a rate-limiting conformational change immediately prior to chemistry. This limitation was removed with findings described herein that for PV polymerase (3Dpol), an RNA-dependent RNA polymerase (RdRp), phosphoryl transfer is partially or completely rate-limiting in Mg²⁺ or Mn²⁺, respectively, and therefore accessible to mechanistic examination.

One proton transfer during the chemistry step of nucleotide incorporation is the removal of the proton from the primer-terminus 3′-OH (and acceptance by an undetermined recipient) during creation of the attacking nucleophile. As described above, universal conservation of a positively charged amino acid residue capable of proton donation at the precise site of bond cleavage and pyrophosphate leaving group formation during phosphoryl transfer suggests that the second catalytically-important proton transfer may involve this active site amino acid residue in a role as general acid catalyst.

### Materials and Methods

Materials: Materials were purchased and prepared as described in Example 1.

Expression and purification of polymerases: 3Dpol RdRp, HIV-1 RT, RB69 DdDp and T7 DdRp were expressed in E. coli and purified as described previously (Castro et al, Proc. Natl. Acad. Sci., 104:4267-4272, 2007) except for the following. Construction of the RB69 DdDp K560L mutant- The K560L mutant was constructed using the pSP72-RB69-pol template and introducing the K560L-encoding mutation into the polymerase gene (gp43) by Quick Change PCR using the forward oligonucleotide RB69-DdDp-K560L-fwd 5′-GCACAAATTAA TCGTCTGTTGCTTATCAACTCAC-3′ (SEQ ID NO:7) and reverse oligonucleotide RB69-DdDp-K560L-rev 5′-GTGAGTTGATAAGCAACAGACGATTAATTTGTGC-3′ (SEQ ID NO:8). The expression vector for RB69 K560A was kindly provided by Dr. William Konigsberg, Yale University.

PV 3D^{pol}-catalyzed nucleotide incorporation experiments: PV 3Dpol stopped-flow and chemical-quench-flow experiments were accomplished as described previously (Castro et al, Proc. Natl. Acad. Sci., 104:4267-4272, 2007). Enzymes, substrates and buffers for proton-inventory experiments were prepared in 100% water or 100% D₂O followed by mixing at the appropriate ratio to obtain 0, 25, 50, 75 or 100% D₂O. Deuterated glycerol was used in all solutions in D₂O. All data collection was performed in the stopped-flow instrument. The pD was used instead of pH for the solutions in D₂O as described above.

Solvent deuterium isotope effect and proton inventory for WT and a K220A HIV-RT were performed essentially as described in Example 1.

RB69 WT DdDp stopped-flow and chemical-quench-flow experiments: RB69 DdDp WT solvent deuterium isotope effect and proton inventory chemical-quench-flow experiments were done essentially as described in Example 1, with reactions quenched in 1 M HCl, neutralized and analyzed as described in Example 1.

Stopped-flow K_{d,app} and kₚₒₗ, solvent deuterium isotope effect and proton inventories for RB69 K560A: Experiments were done using reaction conditions and DNA primer and template essentially as described for WT (Castro et al, Proc. Natl. Acad. Sci., 104:4267-4272, 2007). For K_{d,app} and kₚₒₗ determinations, k_{obs} was obtained using [dATP] of 0.5, 1, 2.5, 5 and 10 mM. For 2.5 and 10 mM dATP reactions, MgSO₄ was supplemented to maintain free Mg²⁺ at 10 mM. Plots of k_{obs} vs. [dATP] were fit with a hyperbola (eq. 2). Solvent deuterium isotope effect and proton inventory experiments were conducted as for WT except [dATP] was 10 mM and MgSO₄ was increased to provide 10 mM free Mg²⁺ over that bound to dATP. Chemical-quench-flow solvent deuterium isotope effect for RB69 K560A-Experiments were done as described above in the stopped-flow except [dATP] was 5 mM. Reactions were quenched with 0.25 M EDTA as described in Example 1.

Chemical-quench-flow K_{d,app}, kₚₒₗ, solvent deuterium isotope effect and proton inventories for RB69 K560L: Experiments were done using reaction conditions and DNA primer and template essentially as described for WT (Castro et al, Proc. Natl. Acad. Sci., 104:4267-4272, 2007). For K_{a,app} and kₚₒₗ determinations, k_{obs} was obtained using [dATP] of 0.3, 1, 3, 6 and 12 mM. For 3, 6 and 12 mM dATP reactions, MgSO₄ was supplemented to maintain free Mg²⁺ at 10 mM. Plots of k_{obs} vs. [dATP] were fit with a hyperbola (eq. 2). Solvent deuterium isotope effect and proton inventory experiments were conducted as for WT except [dATP] was 5 mM and MgSO₄ was increased to provide 10 mM free Mg²⁺ over that bound to dATP. Chemical-quench-flow RB69 K560L reactions were quenched with 0.25 mM EDTA as described in Example 1.

Data analysis: Data analysis was performed as described in Example 1.

### Results

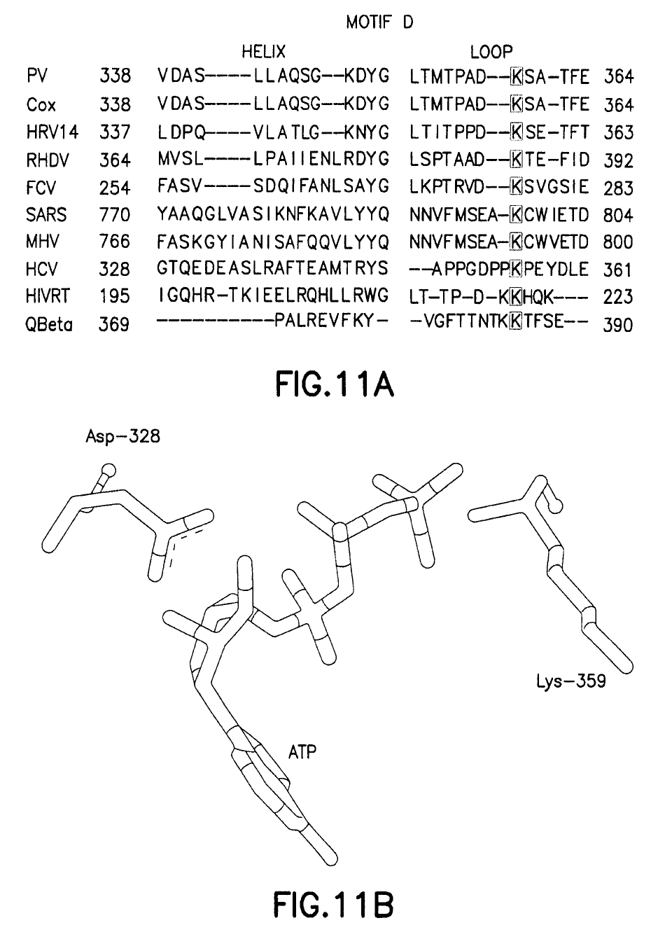

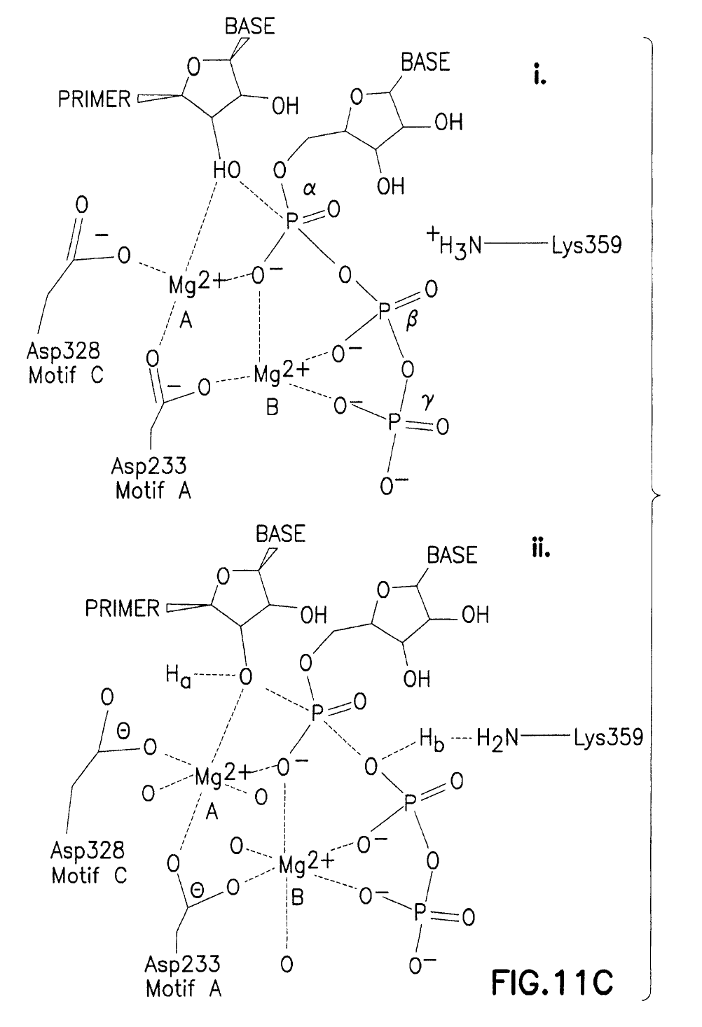

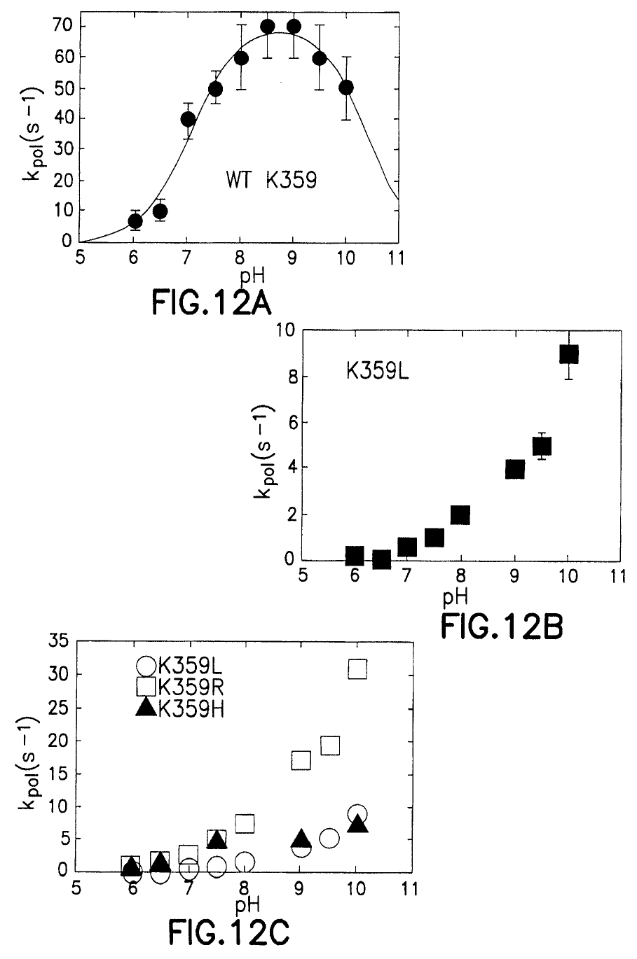

The conserved active site positively charged amino acid residue in PV 3Dpol is Lys-359: Nucleic acid polymerase architecture resembles a cupped right hand consisting of palm, thumb and fingers subdomains. Conserved structural motifs comprise the palm subdomain, the location of the polymerase active site. Alignment of RNA polymerase palm structural motif D amino acid residues shows absolute conservation of a lysine residue (FIG. 11A). In PV 3Dpol, this conserved active site residue is Lys-359 (FIG. 11A). Molecular modeling based on PV 3Dpol crystal structures suggest that 3Dpol Lys-359 interacts intimately with the triphosphate moiety of the bound nucleotide at a site between the α- an β-phosphorus atoms (FIGS. 11B and 11Ci). Experimental data summarized below indicate that the PV 3Dpol Lys-359 serves as a general acid catalyst during the phosphoryl transfer step of nucleotide incorporation by donating its dissociable proton at the site of bond cleavage to the pyrophosphate leaving group (FIG. 11Cii).

Kinetic parameters for 3Dpol position-359 mutants suggest a catalytic role for Lys-359: To evaluate the influence of PV 3Dpol Lys-359 on the phosphoryl transfer step, this residue was changed to Leu, His or Arg (Table 7). K359L 3Dpol with Mg²⁺ as metal-ion co-factor showed a 50-fold reduction in single nucleotide incorporation catalytic rate (kₚₒₗ) relative to WT K359 and a 3-fold weakening in nucleotide binding, as reported by K_{d,app}. These data suggest a profound role for Lys-359 in catalysis and a lesser role in nucleotide binding. The reduced catalytic performance of K359L 3Dpol is consistent with lack of ability to donate a proton by the chemically inert Leu side chain. The reduction, albeit small, in nucleotide binding is consistent with absence of a positive charge on the Leu side chain, presumably important for binding interactions with the negatively-charged nucleotide triphosphate moiety.

TABLE 7
Summary of poliovirus 3Dpol RdRp kinetic parameters

| Enzyme | Metal co-factor | K_{d,app} (μM)^{a} | kₚₒₗ (s⁻¹) | SDKIE^{b} |
|---|---|---|---|---|
| WT Lys 359 | Mg²⁺ | 200 ± 20 | 50 ± 5 | 3 ± 1 |
| WT Lys 359 | Mn²⁺ | 5 ± 1 | 10 ± 1 | 7 ± 1 |
| K359L | Mg²⁺ | 700 ± 80 | 1 ± 0.1 | 2 ± 1 |
| K359L | Mn²⁺ | 2 ± 0.1 | 1 ± 0.1 | 3 ± 1 |
| K359H | Mg²⁺ | 339 ± 12 | 5 ± 0.1 | nd^{c} |
| K359R | Mg²⁺ | 139 ± 1 | 5 ± 0.1 | nd |

^{a}K_{d,app} is for ATP.
^{b}SDKIE is solvent deuterium kinetic isotope effect, calculated as k_{obs} in H₂O/k_{obs} in D₂O at saturating [ATP].
^{c}nd means not determined.

The solvent deuterium kinetic isotope effect (SDKIE) for WT K359 3Dpol was 2-fold larger in the presence of Mn²⁺ than in Mg²⁺, consistent with chemistry being completely rate-limiting in Mn²⁺ and partially rate-limiting in Mg²⁺. In contrast, identical and low kₚₒₗ values and indistinguishable SDKIE values in Mg²⁺ and Mn²⁺ for K359L 3Dpol point to the importance of Lys-359 in catalyis and indicate that chemistry is completely rate-limiting with either metal ion co-factor for this mutant enzyme (Table 7).

Catalytic rates for the K359H and K359L 3Dpol variants were 5-fold faster than for K359L 3Dpol, but 10-fold slower than the WT K359 polymerase (Table 7). Nucleotide binding interactions for the K359H and K359L enzymes were similar to that of WT, consistent with retention of ability of the positively charged His and Arg side chains to bind a negatively-charged incoming nucleotide. However, while both His and Arg have exchangeable protons theoretically available to assist catalysis, their 10-fold diminished catalytic rates relative to the WT K359 enzyme suggests that the active site environment during phosphoryl transfer is not chemically (i.e. matching of pKas, etc.) and/or spatially optimized to make efficient catalytic use of these non-biological residues when at position 359.

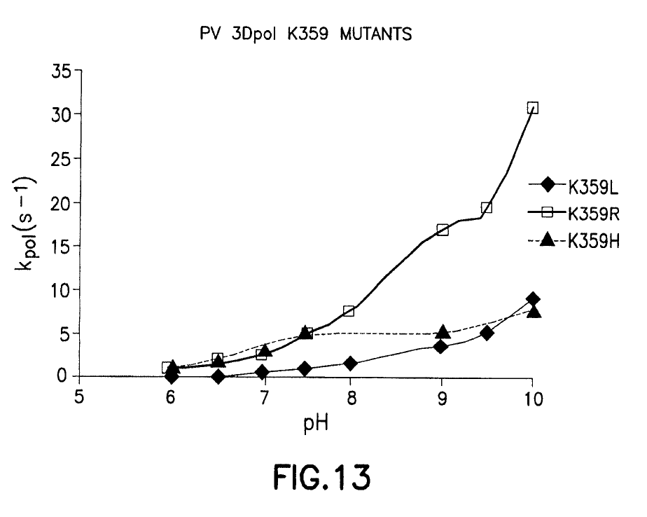

As shown in FIG. 12B, the basic arm of the pH-rate profile for the K359L enzyme has been lost, leaving only one ionizable group influencing phosphoryl transfer. This finding is consistent with proton donation by Lys-359 in a role as a general acid catalyst. The pH-rate profiles for K359H and K359R 3Dpols, superimposed over the profile for the K359L enzyme further support a role for Lys-359 as a proton-donating catalyst (FIG. 13). The K359H enzyme was kinetically superior to K359L at lower pHs, was maximally superior near its pKa of approximately 7.5 (Table 7 and FIG. 13), and then converged in kinetic performance toward that of K359L at higher pHs. The pH-rate behavior of K359H suggests some ability of its dissociable imidazole-group proton to enhance catalysis up to and somewhat beyond its pKa. However, the ability to enhance catalysis beyond that of Leu at position-359 is lost in K359H at higher pH values as the His imidazole becomes completely deprotonated. The K359H pH-rate profile (FIG. 13) lacks a descending basic arm (as seen for WT K359 3Dpol in FIG. 2A) because the pKa associated with transfer of the primer-template 3′-OH proton and the pKa associated with the His imidazole are likely similar enough that they are nearly superimposed on the same region of the pH-rate profile and thus unresolved.

The kinetic behavior of the K359R 3Dpol was similar to that of K359H at lower pH values (FIG. 13). However, at higher and higher pH values the K359R enzyme increasingly exceeded the kinetic performance of K359H and K359L 3Dpols, approaching that of the WT K359 polymerase at pH 10. The K359R 3Dpol pH-rate behavior is consistent with the high pKa of the Arg side chain. At lower pHs, the Arg dissociable proton is minimally unavailable to assist catalysis via proton donation. However, as its pKa is approached, the Arg dissociable proton becomes increasingly available and functions increasingly efficiently as a general acid catalyst. As expected, a basic arm for the K359R pH-rate profile was not observed up to pH 10. Reactions through at least pH 12, an experimental impossibility due to precipitation, would likely be required to observe the basic arm, given that the pKa of the Arg side chain is in the pH 12 range.

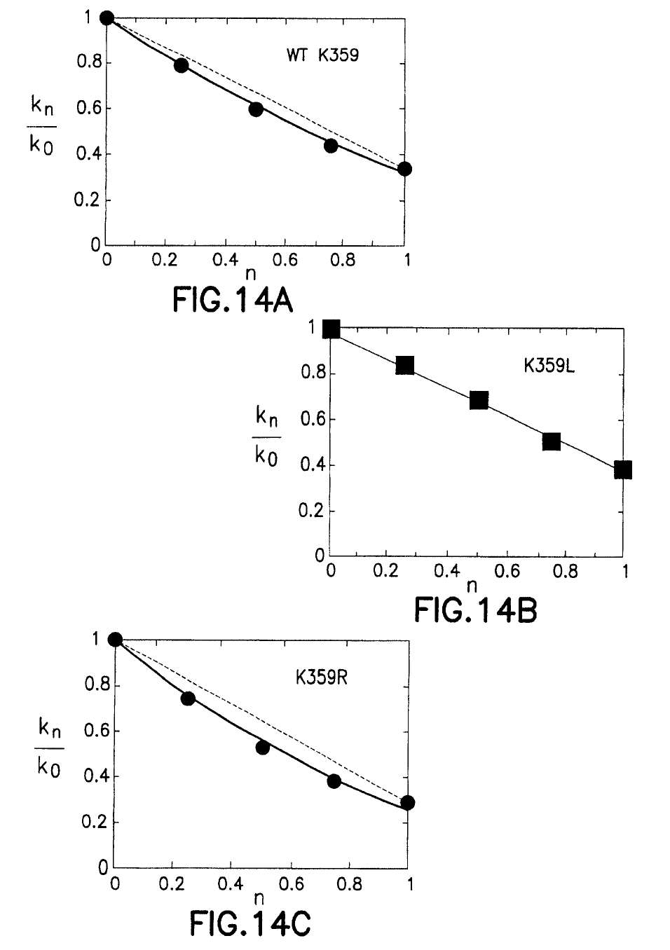

Proton inventories reveal general-acid catalysis by PV 3Dpol Lys-359: Proton inventories for PV WT Lys-359 3Dpol were bowl-shaped, indicating transfer of more than one proton in the transition state of the phosphoryl transfer step of nucleotide incorporation (FIG. 14A). In constrast, proton inventories for the K359L mutant polymerase produced a straight-line plot indicating transfer of only one proton in the transition state. This observation is strong evidence that Lys-359 is the donor of a catalysis-enhancing proton and thus serves as a general-acid catalyst. Consistent with this, proton inventories for the K359R 3Dpol variant were bowl-shaped, indicating some ability for Arg at position-359 position to facilitate catalysis by proton donation, as suggested above by kinetic data (Table 7) and pH-rate behavior (FIG. 13).

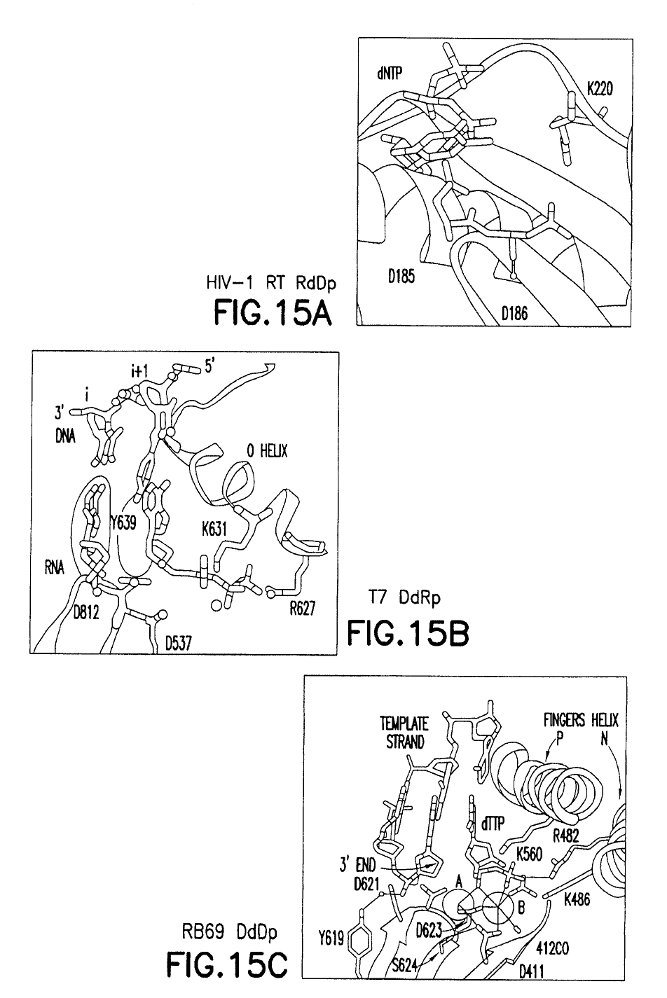

A conserved active site Lys is a general acid catalyst in other polymerase classes: Sequence alignments and high resolution crystal structures permit identification of a conserved Lys residue occupying the same active site functional position as PV 3Dpol Lys-359 in nucleic acid polymerases from other classes. HIV-1 RT RdDp Lys-220 (FIG. 15A), RB69 DdDp Lys-560 (FIG. 15B) and T7 DdRp (often termed RNAP, for RNA polymerase) Lys-631 (FIG. 15C) were investigated. Consistent with findings described above for PV 3Dpol Lys-359 (Table 7), kinetic data suggest a catalytic role for the conserved active site Lys residues in these other polymerases (Table 8). Mutation of the conserved active site Lys to Leu resulted in decreased catalytic rates ranging from approximately two orders of magnitude for HIV-1 RT RdDp K220L and T7 DdRp K631L to over three orders of magnitude for RB69 DdDp K560L. While the impact of the Lys to Leu change on catalytic rates in these enzymes was consistent and severe, the influence on nucleotide binding, as reported by K_{d,app}, was highly variable. Little or no change was seen in HIV-1 RT K220L, a 25-fold decrease in binding affinity was seen in RB69 K560L and a devastating loss of ability to bind nucleotide substrate was seen in T7 DdRp K631L (Table 8). For the latter enzyme, saturation with nucleotide could not be accomplished experimentally. Even 75 mM ATP did not produce catalytic rates in the plateau region of a k_{obs} vs. [ATP] plot. Of interest in the case of RB69 DdDp was the finding that the K560A enzyme was 10-fold kinetically superior to K560L (but still 250-fold inferior to the WT RB69 K560 polymerase). In contrast, nucleotide binding affinity for K560A and K560L were indistinguishable.

TABLE 8
Summary of kinetic parameters for HIV-1 RT RdDp, RB69
DdDp and T7 DdRp

| Polymerase | Variant | K_{d,app} (μM)^{a} | kₚₒₗ (s⁻¹) | SKDIE^{b} |
|---|---|---|---|---|
| HIV-1 RT RdDp | WT K220 | 7 ± 0.5 | 63 ± 1.2 | 2 ± 0.5 |
| HIV-1 RT RdDp | K220L | 5 ± 2 | 0.3 ± 0.3 | 2 ± 0.5 |
| RB69 DdDp | WT K560 | 38 ± 5 | 223 ± 8 | 4 ± 1 |
| RB69 DdDp | K560L | 972 ± 14 | .09 ± .003 | 2 ± 0.5 |
| RB69 DdDp | K560A | 891 ± 4 | 1.3 ± .02 | 2 ± 0.5 |
| T7 DdRp | WT K631 | 297 ± 26 | 58 ± 2 | 5 ± 1 |
| T7 DdRp | K631R | 6400 ± 0.7 | 23 ± 0.9 | nd^{d} |
| T7 DdRp | KK631L | >83000^{c} | >0.67^{c} | 3 ± 0.5^{c} |

^{a}K_{d,app} is for ATP.
^{b}SDKIE is solvent deuterium kinetic isotope effect, calculated as k_{obs} in H₂O/k_{obs} in D₂O at saturating [ATP].
^{c}Values listed for T7 K631L are not actual K_{d,app} and kₚₒₗ but rather represent lower limits for these values because it was impossible to achieve saturation of reactions with ATP. Likewise, the SKDIE was obtained under sub-saturating ATP concentrations.
^{d}nd means not determined

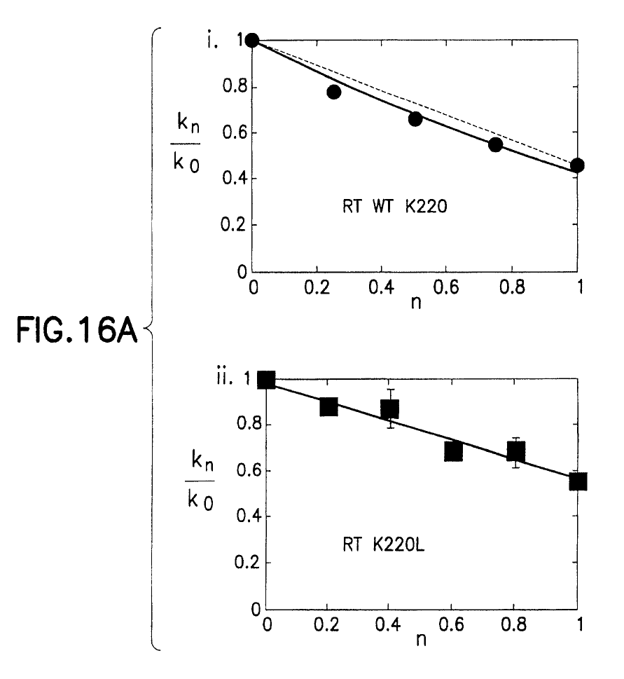

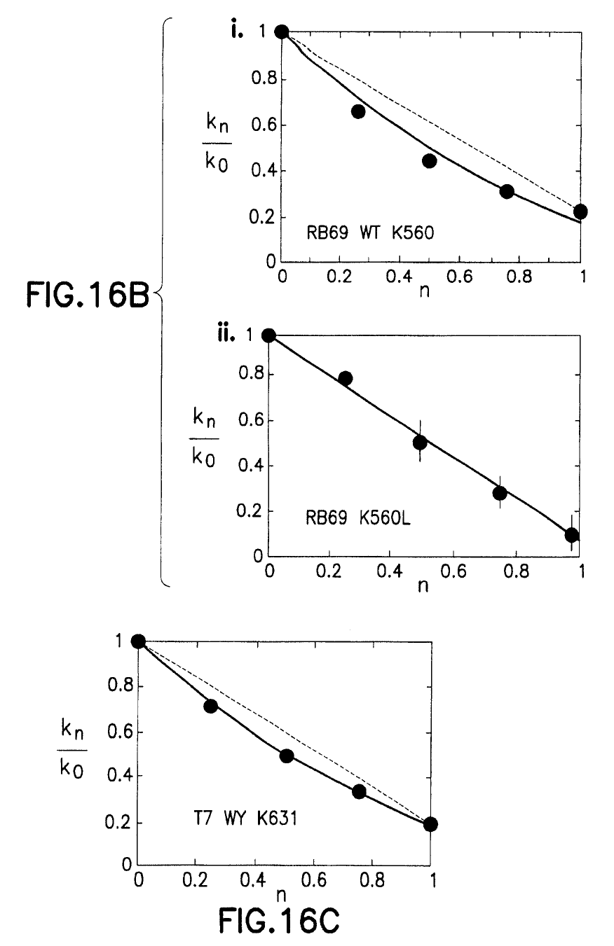

As described above for PV 3Dpol RdRp (FIG. 14), proton inventory plots for HIV-1 RT RdDp and RB69 DdDp reveal a role as catalytic general acid for the conserved active site Lys in these latter two polymerases. Proton inventory plots for WT HIV-RT K220 RdDp, T7 K631 DdRp and RB69 K560 DdDp were bowl-shaped, indicating transfer of more than one rate-influencing proton in the transition state of the phosphoryl transfer reaction during nucleotide incorporation (FIGS. 16Ai, 16Bi and 16C). In contrast, proton inventory plots for HIV-1 RT K220L (FIG. 16Aii) and RB69 K560L (FIG. 16Bii) were linear, indicating transfer of only one rate-influencing proton in the transition state. These data strongly implicate the HIV-RT RdDp and RB69 DdDp conserved active site Lys residues as general-acid catalysts.

A proton inventory for the T7 DdRp K631L polymerase could not be accomplished because, as described above, mutation of Lys to Leu in this enzyme abolished the ability to saturate the enzyme-primer/template binary complex with nucleotide experimentally.

In spite of wide diversity in the biological roles fulfilled by nucleic acid polymerases as agents of DNA and RNA replication and maintenance, the underlying active site architecture and mechanistic steps used to accomplish single nucleotide incorporation are highly conserved. In particular, the catalytic role during phosphoryl transfer has been assigned nearly exclusively to the two active site Mg²⁺ ions. These metal ions position reacting functional groups, facilitate deprotonation of the primer-terminus 3′-OH to create the attacking nucleophile and alleviate negative charge build-up during the transition state.

However, the finding that two transition-state proton transfers influence the rate chemistry reveals the importance of protonic catalysis in the phosphoryl transfer reaction of nucleic acid polymerases. Abstraction of the primer-terminus 3′-OH proton likely comprises one critical proton transaction. Numerous lines of experimental evidence described in this paper converge upon the conclusion that the other critical proton exchange is proton donation by a conserved active site positively charged amino acid residue, usually lysine, acting as a general-acid catalyst. Recent high-resolution x-ray crystal structures reveal that presence of this positively charged active site residue is absolutely conserved and, even more significantly, also conserved is its spatial position near the bridging oxygen between the α- and β-phosphorus atoms of the bound nucleotide triphosphate moiety, the ideal location to facilitate bond cleavage and pyrophosphate leaving group departure. Such specific conservation demands consideration of an extremely important function. Direct participation of a conserved active site basic residue in catalysis has been largely overlooked because 1) metal ion catalysis is clearly profoundly important, 2) a role in nucleotide binding was thought to explain presence of the conserved basic residue and 3) the ability to clearly interrogate the phosphoryl transfer step has been lacking in many model polymerase systems.

In PV 3Dpol RdRp, sequence alignments and molecular modeling identified Lys-359 as the putative active site general acid (FIG. 11). pH-rate profiles of WT K359 3Dpol indicated that two ionizable functional groups influence the rate of phosphoryl transfer (FIG. 12A). Proton inventory assays further supported the transfer of two protons in the transition state for WT 3Dpol (FIG. 14A). Mutation of Lys-359 to chemically inert Leu led to a pH-rate profile lacking a basic arm (FIG. 12B), indicating only one ionizable group influencing chemistry in the K359L enzyme. Consistent with this, proton inventories of K359L 3Dpol showed transfer of only one proton in the transition state (FIG. 12B). Whereas mutation of Lys to Leu at position 359 of PV 3Dpol resulted in only a three-fold reduction in nucleotide binding affinity (the historical role ascribed to this conserved residue before its structural location was revealed), a much more profound 50-fold reduction in catalytic rate was seen (Table 7).

Studies with K359H and K359R 3Dpols are also supportive of a role as catalytic general acid for PV 3Dpol Lys-359. The pH-rate profile of K359H 3Dpol revealed this enzyme to be kinetically superior to the K359L enzyme up to approximately one pH unit beyond the pKa of the His imidazole moiety but to then converge toward the catalytic inefficiency of K359L 3Dpol at higher pHs (FIG. 13). The pH-rate profile of K359R 3Dpol showed this enzyme to be kinetically similar to K359H 3Dpol at lower pHs, but at higher and higher pHs the K359R polymerase performed catalytically better and better until, by pH 10, catalytic rates of K359R approached those of the WT K359 enzyme (FIG. 13). Also consistent with a role as general acid catalyst of 3Dpol Lys-359 was the finding that proton inventories for K359R 3Dpol revealed two proton transfers in the transition state for this polymerase variant (FIG. 14c). Mutation of Lys-359 to His or Arg left nucleotide binding essentially unaffected relative to the WT 3Dpol enzyme but resulted in catalytic rates 10-fold slower than WT K359 and five-fold faster than K359L 3Dpol (Table 7). In summary, experimental data for the PV 3Dpol K359H and K359R polymerase variants suggest that some residual general acid catalytic function exits when His or Arg occupy position 359 but that protonic catalysis occurs with substantially reduced efficiency compared to WT Lys-359. This is likely the result of proton donation limitations due to pKa mismatches between proton donor and acceptor groups and/or spatial misalignments between interacting atoms when the non-biological His or Arg occupy this active site position in PV 3Dpol.

In further support of a role as catalytic general acid for the conserved polymerase active site positively charged amino acid residue is extension of this conclusion described above for PV 3Dpol RdRp to other classes of polymerases. The combination of sequence alignments and high-resolution x-ray crystal structures permitted identification of a conserved active site lysine putative general acid in HIV-1 RT RdDp (K220) (FIG. 15A), RB69 DdDp (K560) (FIG. 15B) and T7 DdRp (K631) (FIG. 15C). In each of these polymerases, mutation of the conserved active site Lys to Leu resulted in large decreases in catalytic rate of approximately two-three orders of magnitude (Table 8). Of interest in the case of RB69 DdDp is the finding that K560A polymerase was an order of magnitude kinetically superior to the K560L variant (but still two orders of magnitude inferior to the WT Lys-560 enzyme) in terms of catalytic rate, whereas the K560A and K560L enzymes were equivalent in nucleotide binding (Table 8). A possible explanation for the kinetic superiority of K560A over K560L is that the small Ala side chain may provide space for stable insertion of a structural water molecule which can contribute catalytically at the site, albeit inefficiently.

Proton inventory plots for WT HIV-1 RT RdDp, RB69 DdDp and T7 DdRp were clearly bowl-shaped, revealing transfer of more than one rate-influencing proton in the transition state of phosphoryl transfer (FIGS. 16Ai, 16Bi and 16C). In contrast, proton inventory plots for the HIV-1 RT K220L and RB69 K560L polymerases were linear, indicating that only one proton was being transferred in the transition state (FIGS. 16Aii and 16Bii). These proton inventory findings were similar to those described above for PV 3Dpol RdRp (FIGS. 14A and 14B). Therefore, in PV 3Dpol RdRp, HIV-1 RT RdDp and RB69 DdDp mutation of the conserved active site Lys to protonically inert Leu eliminated one rate-influencing proton transfer in the transition state of the phosphoryl transfer step and reduced the catalytic rate two-three orders of magnitude.

How nucleic acid polymerases enforce fidelity is currently highly controversial. Lack of ability to saturate T7 DdRp K631L reactions with nucleotide substrate experimentally appears consistent with the observation that in yeast, RNAP II nucleotide binding and catalysis may be coupled by an active site basic residue, His-1085. It has been further suggested that coupling of substrate selection and catalysis may enforce fidelity in RNA polymerases. The T7 RNAP O-helix, which bears Lys-631, may be functionally analogous to the yeast RNAP II trigger loop, which bears His-1085; both are mobile structural elements involved in nucleotide entry into the polymerase active site. The findings reported here that mutation of RB69 Lys-560 to Leu resulted in a 25-fold reduction in K_{d,app} (to go along with a 2500-fold reduction in kₚₒₗ) with dATP as substrate (Table 8) suggests a significant role for this residue in nucleotide binding and a possible coupling with catalysis. However, lack of substantial changes in nucleotide binding upon mutation of the catalytic Lys to Leu in PV 3Dpol RdRp and HIV-1 RdDp, in spite of decreases of approximately two orders of magnitude in catalytic rate (Tables 7 and 8) suggest that coupling of substrate binding and catalysis may not be a feature of these polymerases.

The mechanistic model favored here involves donation of a proton from the active site general acid to the pyrophosphate leaving group to facilitate bond cleavage and leaving group departure. However, it should be noted that complete physical donation of the proton may not be a requirement for successful catalysis and that proton sharing may be sufficient. In summary, multiple lines of evidence described herein collectively argue that a conserved active site positively charged amino acid residue serves an important function as general acid catalyst in nucleic acid polymerases. Proton donation/sharing is the simplest, most fundamental means of dissipating energetically unfavorable negative charge development in enzyme active-site reactions. The findings reported here are important because they reveal a more complete picture of how the catalysis of phosphoryl transfer is accomplished by this universally important group of enzymes. While two-metal-ion catalysis is clearly profoundly important and provides the primary basis for acceleration of nucleotide incorporation, rates imparted by metal-ion-catalysis alone may be inadequate. The additional two-three orders of magnitude of catalytic-rate enhancement supplied by the active site general acid may be necessary to bring nucleotide incorporation rates to a level of biological sufficiency. Indeed, cell culture studies of 3Dpol K359H and K359R mutant polioviruses show substantially-slowed plaque formation by these variants, suggesting that infectivity and pathogenesis may prove to be attenuated due to inadequate rates of viral genome replication.

### Example 3

### Mutant PVs

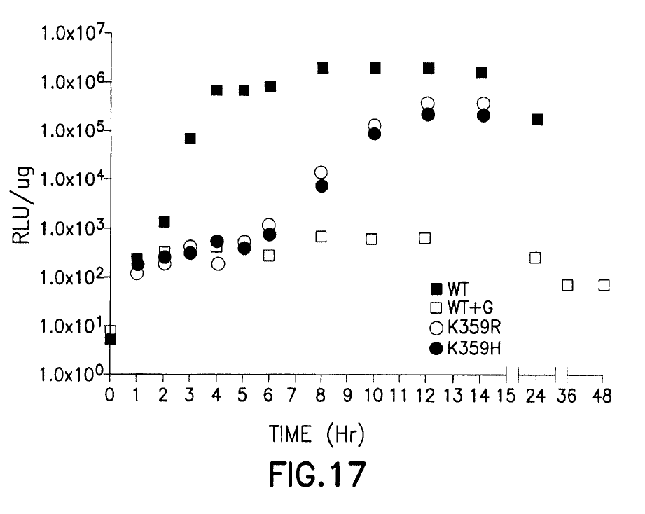

PV mutant viruses K359R and K359H were made and shown to replicate (FIG. 17), and to be infectious and attenuated in cells relative to WT virus. HeLa cells were transfected with wild-type PV RNA, RNA containing the indicated mutations in 3Dpol, or no RNA (mock transfection), diluted 100-fold in PBS, then plated onto a monolayer of untransfected HeLa cells. Plates were covered with agar media and incubated at 37° C. for two days. FIG. 17 illustrates that PV K359R,H subgenomic replicons replicate slower than WT. The subgenomic replicon employed had the capsid-coding region replaced by a luciferase reporter gene. HeLa cells were transfected with a subgenomic replicon RNA containing the WT sequence in the presence of 2 mM GdnHCL (+G) or WT sequence in the absence of GdnHCL. GdnHCL is a reversible inhibitor of PV RNA replication; therefore, the luciferase activity measured in the presence of GdnHCL is the result of translation of the input RNA. In the absence of GdnHCL, transfections were also performed using replicons containing the K359R,H mutations. Transfection reactions were incubated at 37° C. At various times after transfection, cells were processed and luciferase activity evaluated.

### Example 4

### Nucleic Acid Polymerases use a General Acid for Nucleotidyl Transfer

### Results

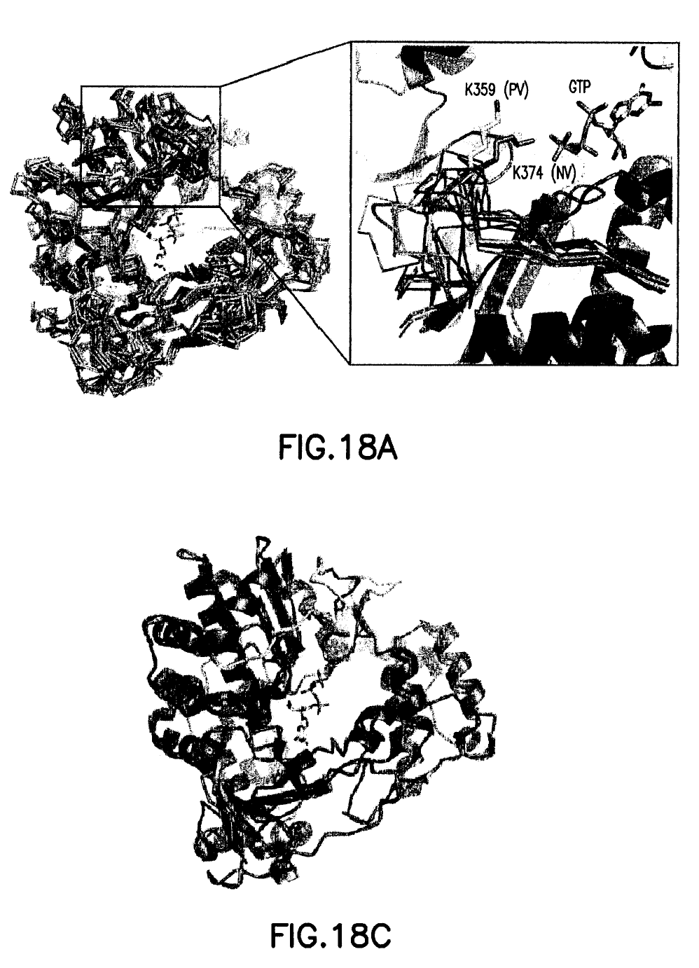

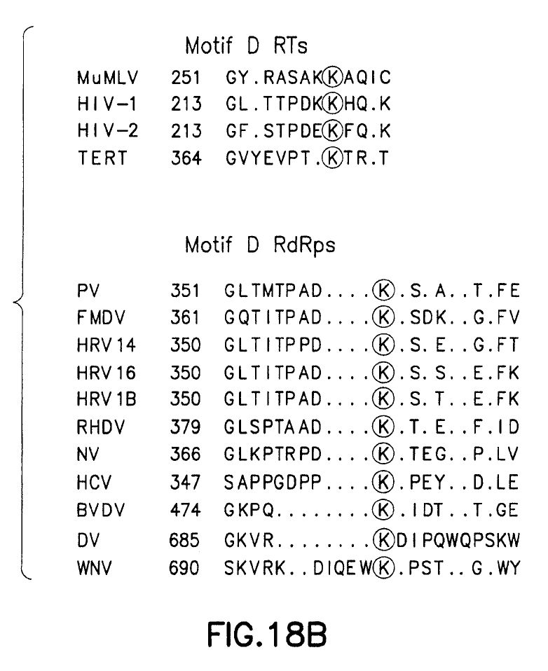

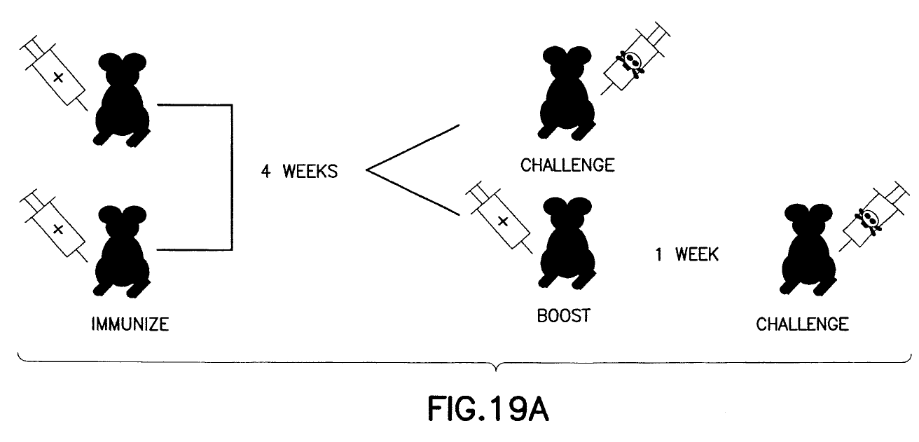

Analysis of the structural model for the RdRp from Norwalk virus (NV) in complex with primed template RNA and nucleotide showed that Lys374 of conserved structural motif D was located in the vicinity of the triphosphate moiety of incoming nucleotide (FIGS. 18A, B). Structural motif D is conserved between RdRps and RTs and has no defined catalytic function (Canard et al., J Biol Chem vol. 274:35768-35776, 1999). Analysis of structure-based sequence alignments showed only one conserved lysine residue in motif D of RdRps and RTs, including the telomere RT, telomerase (TERT) (FIG. 18B). These sequence alignments identified Lys359 of PV RdRp and Lys220 of HIV RT in conserved structural motif D as candidates for the proton donors in these systems (FIG. 18B). Lys219 of HIV RT was ruled out as the putative general acid because of the absence of this residue in the RT from human immunodeficiency virus type 2 (HIV-2) (FIG. 18B). It is well established that the DNA polymerase activity of HIV-2 RT is on par with that observed for HIV RT. Unlike Lys374 of NV RdRp, Lys359 of PV RdRp and Lys220 of HIV RT were not oriented in a position to interact readily with the triphosphate moiety of the incoming nucleotide. Structural motif D is one of the most dynamic elements of the palm subdomain of RdRps and RTs, with the position of this motif varying by as much as 6 A when structures are compared (FIG. 18A, right panel). Solution of the structure of HIV RT in complex with primed template DNA and nucleotide required crosslinking of the enzyme to DNA that could have influenced the orientation of motif D. This possibility is supported by a recently published model for the TERT elongation complex that positions the motif-D lysine in a position to serve as a general acid. Therefore, only biochemical studies could test the possibility that the conserved lysine in motif D functions as a general acid. Identification of candidates for the general acid in DNA-dependent DNA and RNA polymerases was more straightforward. Structural models show clearly that Lys560 in helix P of RB69 DdDp, a B-family polymerase, and Lys631 in helix O of T7 DdRp, an A-family polymerase, are positioned to serve as proton donors for these enzymes.

Derivatives of each of the polymerases described above were produced that contained a leucine residue instead of the candidate lysine proton donor (methods described in Example 5). Leucine was selected instead of alanine to prevent water from binding to the site and serving as a proton donor. For all polymerases tested, the maximal rate constant for nucleotide incorporation (kpol) was reduced by 50-2000 fold for the Leu derivative relative to the wild-type, lysine-containing (wt) enzyme (Table 9). This observation is consistent with a role for the lysine in the rate-limiting step for nucleotide incorporation. For all of the wt polymerases employed here, chemistry is at least partially rate limiting (see Example 1). With the exception of the leucine derivative of T7 DdRp, the kpol values derive from experiments that included a saturating nucleotide concentration (5×K_{d,app}) (methods described in Example 5). In the case of the leucine derivative of T7 DdRp, the highest concentration of nucleotide attainable was two times the K_{d,app} (methods described in Example 5). This circumstance increases the error on the kpol value measured; however, our ability to reach conclusions for the leucine derivative is not affected because the observed 100-fold reduction in kₚₒₗ value for this derivative is much greater than the error of the measurement.

TABLE 9
Kinetic Analysis of PV RdRp, HIV-1 RT, RB69 DdDp and T7 DdRp
supports general acid catalysis in nucleotidyl transfer

| | PV | PV | | | RB69 | RB69 | T7 | T7 |
|---|---|---|---|---|---|---|---|---|
| Parameter | RdRp | RdRp | HIV RT | HIV RT | DdDp | DdDp | DdRp | DdRp |
| measured | WT | K359L | WT | K220L | WT | K560L | WT | K631L |
| kₚₒₗ(s⁻¹) | 50 ± 5 | 1 ± 0.1 | 60 ± 5 | 0.3 ± 0.1 | 200 ± 10 | 0.10 ± 0.01 | 60 ± 5 | 0.6 ± 0.1 |
| K_{d,app} | 200 ± 20 | 700 ± 80 | 7 ± 1 | 5 ± 2 | 40 ± 5 | 1000 ± 100 | 300 ± 30 | 5.0 ± 1.0 × |
| (μM)^{a} | | | | | | | | 10^{4c} |
| SDKIE^{b} | 3.0 ± 0.3 | 2.5 ± 0.3 | 2.2 ± 0.4 | 1.8 ± 0.4 | 4.2 ± 0.2 | 1.8 ± 0.2 | 5.2 ± 0.5 | 2.6 ± 0.5^{c} |
| PI^{d} | 2 | 1 | 2 | 1 | 2 | 1 | 2 | nd^{e} |

Kinetic analysis of K560A RB69 DdDp is consistent with the existence of two forms of the enzyme. Referring to Table 10, K560A DdDp exhibits a reduced affinity for nucleotide substrate and slower rate of catalysis. The kinetic parameters for WT, K560L and K560A RB69 DdDp are shown in Table 10. The maximal rate constant for nucleotide incorporation (Kₚₒₗ) was obtained for dAMP incorporation. Biphasic kinetics observed for K560A DdDP indicated two populations of polymerase-primer/template-deoxynucleotide complex. The results showed that one fraction (the faster fraction) of the K560A DdDp contains a water molecule that can serve as a proton donor; the slower fraction lacks the water molecule. Kinetic analysis results also showed that an ambiguous isotope effect for K560A DdDp was caused by multiple forms of the reactive complex.

TABLE 10
Kinetic Analysis of K560A RB69 DdDp is consistent with the
existence of two forms of the enzyme

| | Parameter | Parameter | Parameter |
|---|---|---|---|
| | measured | measured | measured |
| RB69 Enzyme | K_{d,app} | kₚₒₗ (s⁻¹) | SDKIE |
| WT | 40 ± 5 | 200 ± 10 | 4.2 ± 0.2 |
| K560L | 1000 ± 100 | 0.1 ± 0.01 | 1.8 ± 0.2 |
| K560A | 1000 ± 100 | 1 ± 0.1 | 0.5-20 |

A substantial difference was not observed in the apparent dissociation constant (Kd,app) for the nucleotide substrate measured for the leucine derivatives of PV RdRp or HIV RT (Table 9). This observation is consistent with residues in conserved, structural motif (F) of these enzymes functioning independently in triphosphate binding. In contrast, the K_{d,app} value for nucleotide substrate measured for the leucine derivatives of RB69 DdDp and T7 DdRp increased 25-fold and 167-fold, respectively (Table 9). This observation is consistent with structural studies which show that these lysines interact with the nucleotide substrate.

Unlike many other polymerases, the stability of PV RdRp on its nucleic acid primer-template is not affected by pH, permitting the pH dependence of the kinetics of nucleotide incorporation to be interpreted (methods described in Examples 1 and 5). Consistent with Lys359 of PV RdRp functioning as a proton donor during nucleotidyl transfer, the descending limb of the pH rate profile observed for wt PV RdRp was lost for the K359L derivative. The ionization observed for the K359L derivative likely reflects deprotonation of the 3′-hydroxyl. Theoretical studies of nucleotidyl transfer by rat DNA polymerase beta have suggested that the primer 3′-hydroxyl has a pKa in the 8-9.5 regime. Because the pKa values measured are kinetic pKa values, the lack of equivalence in the pKa value for the K359L derivative to any measured for wt enzyme likely reflects a change in the rate-determining step for nucleotidyl transfer (see Example 1). Chemistry should be solely rate limiting for the K359L derivative whereas a conformational-change step and chemistry are equally rate limiting for wt enzyme.

Theoretical studies of the free energy landscape for phosphoryl transfer almost always show proton transfer in the transition state that would manifest experimentally as a solvent deuterium kinetic isotope effect. The solvent deuterium kinetic isotope effect is defined as the ratio of kpol values obtained when the reaction is performed in H₂O relative to values obtained when the reaction is performed in D₂O (methods described in Example 5). The observation of a solvent deuterium kinetic isotope effect supports a proton-transfer reaction representing one of the microscopic steps reflected in the macroscopic rate constant, kpol. Each wt polymerase exhibited a solvent deuterium kinetic isotope effect of 2-5 (Table 9). This observation is consistent with chemistry contributing to the rate-limiting step(s) reported by the nucleotide-incorporation assay as observed for the wt enzymes (see Example 1). The solvent deuterium kinetic isotope effect measured for each leucine derivative was smaller than observed for the corresponding wt enzyme (Table 9). This observation could be interpreted in one of two ways. First, it is possible that the extent to which chemistry limits nucleotide addition by the leucine derivatives is less than occurs for the wt enzymes. Second, it is possible that fewer protons are being transferred. This latter possibility would be consistent with the candidate lysine residues contributing one proton transfer during nucleotide incorporation.

In order to count the number of protons being transferred during the nucleotidyl-transfer reaction, a proton-inventory experiment (methods described in Examples 1 and 5) was performed. This experiment obtains kₚₒₗ values in the presence of different mole fractions of D₂O (kn). The ratio of kn/k0 (k0 is kₚₒₗ in H₂O) is plotted as a function of mole fraction of D₂O (n). If a single proton is transferred during the reaction, then the data should fit to a line. This experiment was performed for all leucine derivatives except for T7 DdRp derivative, which could not be saturated with nucleotide. The data obtained for all of the leucine derivatives evaluated fit well to a straight line, consistent with a single proton transfer reaction occurring during nucleotidyl transfer for these enzymes. In contrast, the data for the corresponding wt enzymes failed to fall on a line defined by kn/k0 values at n=0 and n=1, consistent with a model for more than one proton transfer reaction occurring during nucleotidyl transfer. More extensive analysis of the proton-inventory experiment for the wt enzymes, including statistical analysis, is described in Example 1, convincingly demonstrating two-proton transfer reactions in the transition state for nucleotidyl transfer by the wt enzymes employed here. These data provide compelling evidence for the use of an active-site amino acid residue as a general acid to protonate the pyrophosphate leaving group to facilitate nucleotidyl transfer.

All of the experiments described to this point have been performed in the presence of Mg²⁺ because this divalent cation is considered to be the biologically relevant cofactor. In the presence of Mg²⁺, chemistry is only partially rate limiting for PV RdRp at pH 7.5¹⁰. In addition, the extent to which chemistry contributes to the observed rate constant for nucleotide addition changes as a function of pH (see Example 1). In the presence of Mn²⁺, however, chemistry is the primary determinant of the rate constant for nucleotide addition by PV RdRp from pH 6.0 to pH 9.0 (see Example 1). Experiments cannot be performed above pH 9.0 in the presence of Mn²⁺ due to precipitation of the metal hydroxide.

The activity of the K359L derivative of PV RdRp in the presence of Mn²⁺ was evaluated. The observed rate constant for nucleotide incorporation was reduced by 10-fold relative to wt enzyme to 1 s⁻¹ without a substantial change in the apparent dissociation constant for nucleotide. The phosphorothioate effect observed for the K359L derivative (6±1) was essentially the same as measured for wt enzyme (7±1), consistent with chemistry remaining as the rate-limiting step. The solvent deuterium kinetic isotope effect observed for the K359L derivative of PV RdRp was 3±1, a value that is more than two-fold lower than the value of 7±1 observed for wt enzyme. These data are consistent with the loss of a proton-transfer reaction as observed in the presence of Mg²⁺. As expected, the dependence of the rate constant for nucleotide incorporation on pH was now essentially identical within the experimentally accessible pH range. These data are consistent with chemistry serving as the rate-limiting step for both the K359L derivative and the wt enzyme in the presence of Mn²⁺. The reduced rate constant for nucleotide incorporation and solvent deuterium kinetic isotope effect observed for the K359L derivative relative to wt enzyme provide additional support for Lys359 being employed as a general acid. It was thus concluded that PV RdRp, and likely the other polymerases, employ general acid catalysis regardless of the divalent cation cofactor employed.

In order to test further the use of a general acid for nucleotidyl transfer, a PV RdRp derivative with Lys359 changed to arginine was produced. In addition to testing the model for general acid catalysis, the ability of an arginine to function at this position as a general acid would be consistent with the use of arginine as a general acid in X-family polymerases like DNA polymerase beta (polβ). An arginine at this position should produce a derivative that functions better than the K359L derivative but still worse than wt enzyme, exhibits unique pH dependence relative to wt enzyme and shows two proton-transfer reactions during nucleotidyl transfer. At pH 7.5, the K359R derivative bound nucleotide well and turned over ˜3-fold faster than the K359L derivative and 20-fold slower than wt enzyme. At pH 10, the kpol value for the K359R derivative had risen to within 2-fold of the maximum value observed for wt enzyme at pH 8.5 and continued to rise. In contrast, the value for the wt enzyme was falling at pH 10. This difference can be explained by the ΔpKa value of 2 between lysine and arginine. The descending limb of the pH rate profile for the K359R derivative should appear at pH values greater than 11. Unfortunately, experiments cannot be performed at pH values higher than 10 because of the insolubility of metal hydroxide. Finally, the solvent deuterium kinetic isotope effect was 3.4±0.1, essentially the same as wt enzyme (Table 9) and proton inventory data fit well to a two proton model. These data strongly support the use of a general acid for nucleotidyl transfer and suggest that both lysine and arginine can serve this function.

Multi-subunit RNA polymerases contain a histidine in the trigger loop that may serve as the general acid for these enzymes. Histidine can function at this position of the PV RdRp. A 10-fold reduction in the kₚₒₗ value was observed relative to wt enzyme with only a modest (50%) increase in the K_{d,app} value for nucleotide at pH 7.5. The pKa value for the ascending limb of the pH rate profile for the K359H derivative was identical to wt enzyme; the descending limb was absent, consistent with a pKa value of 6-7 for histidine.

The general acid of all polymerases appears to be associated with exceptionally dynamic structural elements, and the movement of these elements have been implicated in translocation. A comparison of enzyme-primer/template complexes to enzyme-primer/template-nucleotide complexes revealed movement of the general acid in response to nucleotide binding. It is possible that the protonation-deprotonation cycle of the general acid during catalysis toggles helix O (and its structural equivalents) between the closed (protonated) and open (deprotonated) states, thus driving the translocation reaction. Consistent with this possibility is the observation that changing the general acid to Leu leads to substantial reductions in processivity for all nucleic acid polymerases employed in this study. Severe defects to processive incorporation of nucleotides was observed for leucine derivatives and are consistent with defects to translocation arising from loss of the general acid.

### Methods

Derivatives of all polymerases: PV RdRp, RB69 DdDp, T7 DdRp and HIV RT were prepared by using standard recombinant DNA protocols as described in Example 5. All polymerases were expressed in E. coli and purified as described in Example 1 above. Briefly, cells were transformed with the appropriate plasmid and these cells were used to produce an inoculum for large-scale growth. Gene expression was induced during exponential growth by addition of isopropyl-β-D-thiogalactopyranoside. Induced cells were lysed in appropriate buffers and the enzymes were purified to apparent homogeneity by using standard column chromatography resins and protocols as described in Example 5.

Nucleotide incorporation experiments were performed as described in Examples 1 and 5. SDKIE and PI experiments were performed by monitoring pre-steady state nucleotide incorporation either using the chemical quench flow or stopped-flow instruments. Enzymes, substrates and buffers were prepared in 100% water or 100% D₂O followed by mixing at the appropriate ratio to obtain 0, 25, 50, 75 or 100% D₂O. Deuterated glycerol was used in all solutions in D₂O. The pD was used instead of pH for the solutions in D₂O and was adjusted according to pD=pH+0.4. SDKIE values were calculated as the ratio of kpol values obtained in H₂O divided by that obtained in D₂O. PI plots consisted of kn/kH₂O as a function of n where kn is the observed rate constant for nucleotide incorporation at a particular mole fraction of D₂O, kH₂O is the observed rate constant for nucleotide incorporation in H₂O and n is the solvent mole fraction of D₂O. Proton-inventory data were fit to the modified Gross-Butler equation for a two-proton-transfer model (Venkatasubban, K. S. & Schowen, R. L. CRC Crit Rev Biochem vol. 17, 1-44:1984) or for a one-proton-transfer model (Venkatasubban, K. S. & Schowen, R. L. CRC Crit Rev Biochem vol. 17, 1-44:1984).

Observed rate constants (k_{obs}) for nucleotidyl transfer at various concentrations of nucleotide were obtained by fitting product-versus-time data to an equation defining a single exponential. Values for K_{d,app} and kₚₒₗ were obtained by fitting k_{obs}-versus-[NTP] data to an equation defining a hyperbola. Data were fit by nonlinear regression using the program KaleidaGraph (Synergy Software, Reading, Pa.). Specific equations employed are described in Example 5.

### Example 5

### Supplementary Methods

[γ-³²P]ATP (>7000 Ci/mmol) was purchased from MP Biomedical; [α-³²P] ATP (3000 Ci/mmol was from Perkin Elmer-New England Nuclear; nucleoside 5′-triphosphates, (ultra pure solutions), were from GE-Amersham Pharmacia Biotech, Inc; D₂O was purchased from Isotec (Miamisburg, Ohio); deuterated glycerol was from Cambridge Isotope Laboratories (Andover, Mass.); all RNA oligonucleotides were from Dharmacon Research, Inc. (Boulder, Col.); all DNA oligonucleotides were from Integrated DNA Technologies (Coralville, Iowa); T4 polynucleotide kinase was from New England Biolabs, Inc; all other reagents were of the highest grade available from Sigma, Fisher or VWR.

Introduction of the K359L mutation into the poliovirus (PV) 3D gene required overlap extension PCR by using oligos 1-4 (Table 11) and the expression vector, pET26Ub-PV 3D¹, as the template. First, two PCR fragments were amplified using oligonucleotides 1, 3 and 2, 4 (Table 11) encoding the mutation K359L. These two fragments were then used as templates for the amplification of a new fragment using oligonucleotides 1 and 4 (Table 11). The PCR product, 3D K359L, was then cloned into pET26Ub-PV-3D using KpnI and BamHI sites. Introductions of the K359R and K359H mutations were performed essentially as described above for K359L, except oligonucleotides 5-8 were used encoding the mutations K359R and K359H. The QuikChange® site-directed mutagenesis kit (Stratagene) was employed to introduce the mutation, K220L, into HIV RT-coding sequence with oligonucleotides 9 and 10 (Table 11). The QuikChange® site-directed mutagenesis kit (Stratagene) was employed to introduce the mutation, K560L, into RB69 DdDp-coding sequence with oligonucleotides 11 and 12 (Table 11). The QuikChange® site-directed mutagenesis kit (Stratagene) was employed to introduce the mutation, K631 L, into T7 DdRp-coding sequence with oligonucleotides 13 and 14 (Table 11). Wild-type (wt) and K359H, K359L and K359R 3D^{pol} derivatives were expressed and purified as described above for wild-type. HIV RT wt and K220L polymerases were expressed and purified as described previously (Le Grice, S. F. & Gruninger-Leitch, Eur J Biochem vol. 187:307-314, 1990).

TABLE 11
Oligonucleotides Used
Restriction sites are bold;
nucleotide changes are lower case.

| No. | Name | Sequence |
|---|---|---|
| 1 | PV-3D-KpnI-for | 5′-ATC TGG CTG CTC AGG TAC CTC |
| | | AAT TTT TAA CTC AA-3′ |
| | | SEQ ID NO: 45 |
| 2 | pET26-PV-3D- | 5′-GCG GAA TTC GGA TCC TTA CTA |
| | BamHI-EcoRI-rev | AAA TGA GTC AAG CCA A-3′ |
| | | SEQ ID NO: 46 |
| 3 | PV-3D-K359L-for | 5′-ATG ACT CCA GCT GAC ctg TCA |
| | | GCT ACA TTT GAA ACA-3′ |
| | | SEQ ID NO: 47 |
| 4 | PV-3D-K359L-rev | 5′-TGT TTC AAA TGT AGC TGA cag |
| | | GTC AGC TGG AGT CAT-3′ |
| | | SEQ ID NO: 48 |
| 5 | PV-3D-K359R-for | 5′-ATG ACT CCA GCT GAC cgt TCA |
| | | GCT ACA TTT GAA ACA-3′ |
| | | SEQ ID NO: 49 |
| 6 | PV-3D-K359R-rev | 5′-TGT TTC AAA TGT AGC TGA acg |
| | | GTC AGC TGG AGT CAT-3′ |
| | | SEQ ID NO: 50 |
| 7 | PV-3D-K359H-for | 5′-ATG ACT CCA GCT GAC cAc TCA |
| | | GCT ACA TTT GAA ACA-3′ |
| | | SEQ ID NO: 51 |
| 8 | PV-3D-K359-rev | 5′-TGT TTC AAA TGT AGC TGA gTg |
| | | GTC AGC TGG AGT CAT-3′ |
| | | SEQ ID NO: 52 |
| 9 | HIV RT-K220L-for | 5′-CC ACA CCA GAC AAA ctA CAT |
| | | CAG AAA GAA CCT-3′ |
| | | SEQ ID NO: 53 |
| 10 | HIV RT-K220L-rev | 5′-AGG TTC TTT CTG ATG Tag TTT |
| | | GTC TGG TGT GG-3′ |
| | | SEQ ID NO: 54 |
| 11 | RB69-K560L-for | 5′-GCA CAA ATT AAt CGT ctg TTG |
| | | CTT ATC AAC TCA C-3′ |
| | | SEQ ID NO: 55 |
| 12 | RB69-K560L-rev | 5′-G TGA GTT GAT AAG CAA cag |
| | | ACG aTT AAT TTG TGC-3′ |
| | | SEQ ID NO: 56 |
| 13 | T7-K631L-for | 5′-ACT CGC AGT GTG ACT ctg CGT |
| | | TCA GTC ATG ACG CTG-3′ |
| | | SEQ ID NO: 57 |
| 14 | T7-K631-rev | 5′-CAG CGT CAT GAC TGA ACG cag |
| | | AGT CAC ACT GCG AGT AAC-3′ |
| | | SEQ ID NO: 58 |

RB69 wt and K560L polymerases were expressed in BL21 (DE3) cells. Frozen cells were thawed and suspended in lysis buffer (100 mM KPO₄ pH 8.0, 20% glycerol, 20 mM DTT, 0.5 mM EDTA pH 8.0, 2.8 μg/mL pepstatin A, 2.0 μg/mL leupeptin) and lysed by passing through a French press at 1000 psi. Immediately after lysis, NP-40 was added to 0.1% (v/v) and PMSF was added to 2 mM. Polyethyleneimine (PEI) was added drop wise at 4° C. to a final concentration of 0.25% (v/v) and stirred for 30 mM. This suspension was then centrifuged at 75,000×g for 30 min at 4° C. The supernatant was decanted and collected, and ground ammonium sulfate powder was slowly added to 60% saturation at 4° C. and stirred for 30 min. The ammonium sulfate precipitate was suspended in Buffer A (50 mM Tris pH 8.0, 20% glycerol, 10 mM DTT, 0.1% NP-40) and dialyzed overnight against 1 L Buffer A containing 75 mM NaCl using a 12-14,000 Da MWCO membrane. After dialysis, the sample was diluted in Buffer A to a final NaCl concentration of 50 mM. The sample was loaded onto a phosphocellulose column (approximately 1 mL bed volume/25 mg total protein) at a flow rate of 1 mL/min. The column was washed with ten column volumes of Buffer A containing 50 mM NaCl and the protein was eluted with Buffer A containing 150 mM NaCl. Fractions were pooled based upon their purity on 8% SDS-PAGE gels. The pooled fractions were diluted in Buffer A to a final salt concentration of 50 mM. The sample was loaded onto a Q-Sepharose column (1 mL bed volume/40 mg of protein) at 1 mL/min. The column was washed with ten column volumes of Buffer A containing 50 mM NaCl and the protein eluted with Buffer A containing 150 mM NaCl. Protein-containing fractions were pooled as before. The protein was concentrated using a small (0.5 mL) Q-Sepharose column. Loading and washing of the column was as described above. Elution was performed with Buffer A containing 500 mM NaCl.

Purification of T7 wt and K631L polymerases was done using the same protocol as for the RB69 enzymes with the following modifications: 1. Ammonium sulfate was added to 40% saturation. 2. The phosphocellulose column was eluted using a linear gradient (6 column volumes) from 50 mM-700 mM NaCl in Buffer A. 3. The Q-Sepharose column was loaded and washed, and the protein eluted using a linear gradient (6 column volumes) from 50 mM-400 mM NaCl in Buffer A. Protein-containing fractions were pooled as before. Additional steps to concentrate the protein were not necessary.

Pre-steady state nucleotide incorporation stopped-flow experiments were performed using a Model SF-2001 stopped-flow apparatus (Kintek Corp., Austin, Tex.) equipped with a water-bath. All reactions were performed at 30° C. 3D^{pol} was diluted into enzyme buffer (50 mM HEPES, pH 7.5, 10 mM 2-mercaptoethanol, 60 μM ZnCl₂, and 20% glycerol) immediately prior to use. The volume of enzyme added to any reaction was always equal to one-twentieth the total reaction volume. Reactions were performed in MTCN buffer by incubating 1 μM enzyme with 1 μM sym/sub RNA primer-template³ (0.5 μM duplex) in 1 mM HEPES pH 7.5, 10 mM 2-mercaptoethanol, 60 μM ZnCl₂ and 5 mM MgCl₂ at room temperature for 3 minutes, allowing equilibration to 30° C. and then rapidly mixing with ATP in a solution containing 2×MTCN buffer, 10 mM 2-mercaptoethanol, 60 μM ZnCl₂ and 5 mM MgCl₂ at the pH or pD indicated for individual experiments. 1×MTCN buffer is: 50 mM MES, 25 mM TRIS, 25 mM CAPS and 50 mM NaCl. For reactions with a final ATP concentration higher than 1 mM, the amount of free Mg²⁺ was kept constant by increasing the amount of MgCl₂ in the reaction to 5 mM plus the ATP concentration above 1 mM. For example, in a reaction containing 5 mM ATP, the final Mg²⁺ concentration was adjusted to 9 mM. Values for kpol in pH-rate profiles were obtained by first determining kobs, the observed first order rate constant for single nucleotide incorporation, across a range of [ATP] and then fitting plots of kobs dependence on [ATP] to eq 2 (see below) to obtain estimates of Kd,app and kpol. The excitation wavelength used was 313 nm. Fluorescence emission was monitored by using a 370 nm cut-on filter (model E370LP, Chroma technology corp., Rockingham, Vt. Rate constants for nucleotide incorporation were obtained by using the data analysis software of the instrument.

Pre-steady state nucleotide incorporation rapid mixing/quenching experiments were performed by using a Model RQF-3 chemical-quench-flow apparatus (KinTek Corp., Austin, Tex.). 3D^{pol}-sym/sub complexes and all buffer solutions were prepared in the same way as for the stopped-flow experiments described above. Reactions were quenched by addition of HCl to a final concentration of 1 M. Immediately after the addition of HCl, the solution was neutralized by addition of 1 M KOH and 300 mM Tris base (final concentration). Rapid-quench product analysis was done after fractionation on denaturing PAGE gels. An equal volume of loading/quenching dye (85% formamide, 0.025% bromophenol blue, and 0.025% xylene cyanol, 90 mM EDTA) was added to 10 μl of the quenched reaction mixtures and heated to 70° C. for 2-5 min prior to loading 5 μl on a denaturing 23% polyacrylamide gel containing 1×TBE (2 mM EDTA, 89 mM boric acid, 87 mM Tris) and 7 M urea. Electrophoresis was performed in 1×TBE at 90 W. Gels were visualized by using a phosphorimager and quantified by using the ImageQuant software (Molecular Dynamics).

PV 3Dpol SDKIE and PI experiments were accomplished by monitoring pre-steady state nucleotide incorporation in the stopped-flow instrument. Enzymes, substrates and buffers were prepared in 100% water or 100% D₂O followed by mixing at the appropriate ratio to obtain 0, 25, 50, 75 or 100% D₂O. Deuterated glycerol was used in all solutions in D₂O. The pD was used instead of pH for the solutions in D₂O and was adjusted according to pD=pH+0.4⁴. SDKIE values were calculated as the ratio of kpol values obtained in H₂O divided by that obtained in D₂O. PI plots consisted of kn/kH₂O as a function of n where kn is the observed rate constant for nucleotide incorporation at a particular mole fraction of D₂O, kH₂O is the observed rate constant for nucleotide incorporation in H₂O and n is the solvent mole fraction of D₂O. Proton inventory experimental data were fit to equations 5 (two-proton transfer model) or 6 (one- proton transfer model).

HIV-RT pre-steady state nucleotide incorporation experiments were done in the chemical quench flow instrument at 37° C. essentially as described in Gotte et al., (J Virol. Vol 74:3579-3585, 2000) with some modifications: 2 μM ³²P-labeled DNA primer was annealed with 2.2 μM DNA template, and incubated with 4 μM RT in a solution containing no metals. The reaction was started by addition of 200 μM dATP in reaction buffer containing 10 mM MgCl₂. After mixing, reactant concentrations were reduced by 50%. Product analysis was performed essentially as described for PV 3D^{pol} with the following modifications. 1. Unlabeled DNA primer was added to the loading/quenching dye at a concentration 100-fold that of the labeled primer. A 15% polyacrylamide gel containing 40% formamide was used. SDKIE and PI data were evaluated as described above for PV 3Dpol.

RB69 wt K560 pre-steady state experiments were done in the stopped-flow instrument at 25° C. essentially as described in Example 1. The assays were done in 1×MTCN pH 7.5, 10 mM MgSO₄, 1 mM dATP with 1 μM RB69 enzyme and 150 nM primer-template substrate. RB69 K560L experiments were done in the chemical quench flow instrument with reaction components as described above for WT except using 5 mM dATP and 14 mM MgSO₄. The solvent deuterium isotope effect and proton inventory experiments were performed and evaluated essentially as described above for PV 3D^{pol}.

T7 wt K631 pre-steady state nucleotide incorporation experiments were done in the stopped-flow instrument at 30° C. essentially as described above for poliovirus 3D^{pol} with minor modifications as described in Example 1. One μM RNA primer was annealed to 1 μM DNA template, and then incubated with 1.6 μM T7 for 10 minutes at room temperature in 1 mM Hepes pH 7.5, 10 mM BME, and 5 mM MgCl₂.The reaction was started by mixing the T7-primer-template complex with 3 mM ATP in 2×MTCN pH 7.5, 10 mM BME, 9 mM MgCl₂ and 200 mM NaCl. The solvent deuterium isotope effect and proton inventory experiments were performed and evaluated essentially as described above for poliovirus 3D^{pol}. T7 K631 L pre-steady state nucleotide incorporation experiments were done in the stopped-flow instrument with the goal of obtaining estimates of kpol, the maximal rate constant for nucleotide incorporation and K_{d,app}, the apparent binding constant for dATP, as described above for PV 3Dpol. However complete saturation with ATP was not achievable because of precipitation within the reaction mixture above [ATP] of 75 mM. Proton inventory experiments were therefore not performed with T7 K631L due to inability to saturate the reactions with ATP.

Time courses at fixed nucleotide concentrations were fit to the single exponential function

[product]=A×exp(−kobs×t)+C (1)

where A is the amplitude of the burst, kobs is the observed first-order rate constant describing the burst, t is the time, and C is a constant. The apparent binding constant (Kd,app) and maximal rate constant for nucleotide incorporation (kpol) were determined using the equation:

$$kobs = \frac{kpol [ NTP ]}{Kd , app + [ NTP ]} \qquad ( 2 )$$

The pKa values for the pH dependence of kpol were obtained by fitting to a model describing two ionizable groups⁹:

$$kpol = \frac{kind}{1 + {10}^{pKa 1 - pH} + {10}^{pH - pKa 2}} \qquad ( 3 )$$

or one-ionizable group⁹

$$kpol = \frac{kind}{1 + {10}^{Pka 1 - pH}} \qquad ( 4 )$$

where kind is the pH-independent rate constant.
Proton-inventory data were fit to the modified Gross-Butler equation for a two-proton-transfer model:

$$\frac{kn}{k H 2 O} = ( 1 - n + n \times φ1 ) ( 1 - n + n \times φ2 ) \qquad ( 5 )$$

or for a one-proton-transfer model:

$$\frac{kn}{k H 2 O} = ( 1 - n + n \times φ ) \qquad ( 6 )$$

where kn is the observed rate constant at the different percentages of D₂O, kH2O is the observed rate constant in water, n is the mole fraction of D₂O and Φ is the inverse of the isotope effect for each ionizable group. Data were fit by nonlinear regression using the program KaleidaGraph (Synergy Software, Reading, Pa.). Equilibrium and rate constants reported were determined independently at least twice. Error was propagated through products and quotients by using equation 7:

$$Δ z = \sqrt{{( \frac{Δ x}{x} )}^{2} + {( \frac{Δ y}{y} )}^{2}} \times z \qquad ( 7 )$$

where x and y are the primary data, z is the product or quotient of x and y, and Δx, Δy and Δz are the corresponding error values (Aikens et al., Principles and techniques for an integrated chemistry laboratory, (Waveland Press, Prospect Heights, Ill. 1984).

### Example 6

### Attenuated Virus Vaccines

Challenge experiments were performed in mice. In the first experiment, it was found that PV encoding the K359H mutation confers partial protection to lethal doses of wild-type PV. In this experiment, 15 cPVR mice were immunized, intramuscularly (IM), with 1×10⁵ plaque forming units (PFU) of 3D-P3565-K359H PV. Four weeks after immunization, all animals were challenged IM with 5LD₅₀, 5×10⁶ PFU, of wild-type PV and monitored for signs of paralysis. The percent protection is shown in the table below.

TABLE 12
Percent Protection in Mice Challenge Experiment

| | % protection |
|---|---|
| Immunizing virus | (4 weeks, n = 15) |
| P356S-K359H PV | 26 |

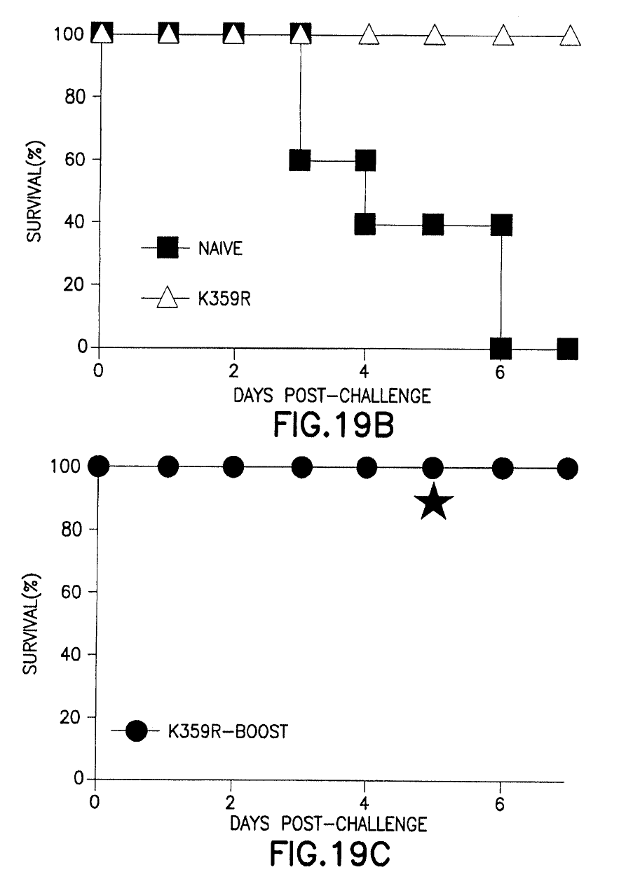

Another challenge experiment was performed which conclusively showed that in addition to PV encoding the K359H mutation, a PV encoding the K359R mutation is an effective vaccine. An illustration of the vaccination protocol used in this challenge experiment is shown in FIG. 19A. The dose of PV that caused disease in the 50% of the animals (LD₅₀) was determined. Animals were challenged with amounts of PV 5-10-fold higher than the LD₅₀, which was shown empirically to be sufficient to cause disease in all animals in no more than five days. In this experiment, five cPVR mice were immunized intraperitoneally (IP), with 3D-K359R PV. Four weeks after immunization, all animals were challenged IP with 5LD₅₀, 10⁸ PFU, of wild-type PV and monitored for signs of paralysis. A second set of five cPVR mice were immunized IP with with 5×10⁵ PFU of 3D-K359R. Four weeks post-immunization, all animals were boosted with another dose of 5×10⁵ PFU of 3D-K359R. One week after boost, animals were challenged IP with 10⁸ PFU of wild-type PV and monitored for signs of paralysis. These data are shown in the graphs of FIGS. 19B and 19C, and provide irrefutable evidence that K359R PV can serve as a vaccine strain.

### Other Embodiments

Any improvement may be made in part or all of the compositions and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Although modified polymerases are described herein having a substitution at a lysine residue in the active site of the polymerase, a modified polymerase as described herein includes mutations, deletions, and substitutions of proton-accepting and proton-donating amino acid residues. Any molecule that changes the activity (e.g., rate of replication, fidelity, etc.) of the polymerase can be utilized. Moreover, any combination of the above-described elements in all possible variations thereof is encompassed by the invention unless otherwise indicated herein or otherwise clearly contraindicated by context.

What is claimed is:

1. A vaccine for a pathogenic virus comprising:
(a) an effective amount of attenuated virus for providing protection from challenge with the pathogenic virus, the attenuated virus comprising a polymerase gene that encodes a polymerase, the polymerase gene having a modification that results in a substitution of a lysine residue to a histidine or arginine residue, the lysine residue capable of functioning as a general acid catalyst during a phosphoryl transfer step of a nucleotide incorporation reaction and located on helix O of A-family polymerases, helix P of B-family polymerases and on the loop of structural motif D of RNA-dependent RNA polymerases and reverse transcriptases,
wherein the substitution causes the polymerase to have increased fidelity compared to a polymerase encoded by a wild-type polymerase gene not having the modification; and
(b) a pharmaceutically acceptable carrier.

2. The vaccine of claim 1, wherein the pathogenic virus is poliovirus and the polymerase is a poliovirus polymerase and the lysine residue is at position 359 of an amino acid sequence encoded by the nucleic acid sequence of SEQ ID NO:9.

3. The vaccine of claim 1, wherein the pathogenic virus is an influenza virus and the polymerase is an influenza polymerase and the lysine residue is at position 481 of an amino acid sequence encoded by the nucleic acid sequence of SEQ ID NO:10.

4. The vaccine of claim 1, wherein the pathogenic virus is human immunodeficiency virus 1 (HIV-1) and the polymerase is an HIV-1 reverse transcriptase and the lysine residue is at position 220 of an amino acid sequence encoded by the nucleic acid sequence of SEQ ID NO:11.

5. The vaccine of claim 1, wherein the polymerase is an A-family polymerase.

6. The vaccine of claim 1, wherein the polymerase is a B-family polymerase.

7. The vaccine of claim 1, wherein the polymerase is a DNA-dependent DNA polymerase.

8. The vaccine of claim 1, wherein the polymerase is an RNA-dependent RNA polymerase.

9. The vaccine of claim 1, wherein the polymerase is a reverse transcriptase.

10. The vaccine of claim 1, wherein the vaccine further comprises an adjuvant.

11. A method of protecting a subject against a pathogenic virus comprising administering to the subject an effective amount of a vaccine for the pathogenic virus comprising (a) an attenuated virus comprising a polymerase gene that encodes a polymerase, the polymerase gene having a modification that results in a substitution of a lysine residue to a histidine or arginine residue, the lysine residue capable of functioning as a general acid catalyst during a phosphoryl transfer step of a nucleotide incorporation reaction and located on helix O of A-family polymerases, helix P of B-family polymerases and on the loop of structural motif D of RNA-dependent RNA polymerases and reverse transcriptases,
wherein the substitution causes the polymerase to have increased fidelity compared to a polymerase encoded by a wild-type polymerase gene not having the modification; and
(b) a pharmaceutically acceptable carrier.

12. The method of claim 11, wherein the pathogenic virus is poliovirus and the polymerase is a poliovirus polymerase and the lysine residue is at position 359 of an amino acid sequence encoded by the nucleic acid sequence of SEQ ID NO:9.

13. The method of claim 11, wherein the pathogenic virus is an influenza virus and the polymerase is an influenza polymerase and the lysine residue is at position 481 of an amino acid sequence encoded by the nucleic acid sequence of SEQ ID NO:10.

14. The method of claim 11, wherein the pathogenic virus is HIV-1 and the polymerase is an HIV-1 reverse transcriptase and the lysine residue is at position 220 of an amino acid sequence encoded by the nucleic acid sequence of SEQ ID NO:11.

15. The method of claim 11, wherein the polymerase is an A-family polymerase.

16. The method of claim 11, wherein the polymerase is a B-family polymerase.

17. The method of claim 11, wherein the polymerase is a DNA-dependent DNA polymerase.

18. The method of claim 11, wherein the polymerase is an RNA-dependent RNA polymerase.

19. The method of claim 11, wherein the polymerase is a reverse transcriptase.

20. The method of claim 11, wherein the vaccine further comprises an adjuvant.

21. A method of inducing an immune response to a pathogenic virus antigen in a subject comprising administering to the subject an effective amount of a vaccine for the pathogenic virus comprising (a) an attenuated virus comprising a polymerase gene that encodes a polymerase, the polymerase gene having a modification that results in a substitution of a lysine residue to a histidine or arginine residue, the lysine residue capable of functioning as a general acid catalyst during a phosphoryl transfer step of a nucleotide incorporation reaction and located on helix O of A-family polymerases, helix P of B-family polymerases and on the loop of structural motif D of RNA-dependent RNA polymerases and reverse transcriptases,
wherein the substitution causes the polymerase to have increased fidelity compared to a polymerase encoded by a wild-type polymerase gene not having the modification; and
(b) a pharmaceutically acceptable carrier,
wherein administration of the vaccine to the subject results in prevention of disease from the pathogenic virus.

22. The method of claim 21, wherein the pathogenic virus is poliovirus and administration of the vaccine prevents paralysis in the subject.

23. The method of claim 21, wherein the pathogenic virus is influenza or human immunodeficiency virus (HIV).

24. The method of claim 21, wherein the vaccine further comprises an adjuvant.
